SEQUENCE LISTING (24 sequences)

SEQ ID NO: 1 - protein, 88 aa, Escherichia coli
MALLDFFLSRKKNTANIAKERLQIIVAERRRSDAEPHYLPQLRKDILEVICKYVQIDPEMVTVQLEQKDGDISILELNVTLPEAEELK

SEQ ID NO: 2 - protein, 231 aa, Escherichia coli
MSNTPIELKGSSFTLSVVHLHEAEPKVIHQALEDKIAQAPAFLKHAPVVLNVSALEDPVNWSAMHKAVSATGLRVIGVSGCKDAQLKAEIEKMGLPILTEGKEKAPRPAPTPQAPAQNTTPVTKTRLIDTPVRSGQRIYAPQCDLIVTSHVSAGAELIADGNIHVYGMMRGRALAGASGDRETQIFCTNLMAELVSIAGEYWLSDQIPAEFYGKAARLQLVENALTVQPLN

SEQ ID NO: 3 - protein, 270 aa, Escherichia coli
MARIIVVTSGKGGVGKTTSSAAIATGLAQKGKKTVVIDFDIGLRNLDLIMGCERRVVYDFVNVIQGDATLNQALIKDKRTENLYILPASQTRDKDALTREGVAKVLDDLKAMDFEFIVCDSPAGIETGALMALYFADEAIITTNPEVSSVRDSDRILGILASKSRRAENGEEPIKEHLLLTRYNPGRVSRGDMLSMEDVLEILRIKLVGVIPEDQSVLRASNQGEPVILDINADAGKAYADTVERLLGEERPFRFIEEEKKGFLKRLFGG

SEQ ID NO: 4 - protein, 164 aa, Bacillus subtilis
MPLTPNDIHNKTFTKSFRGYDEDEVNEFLAQVRKDYEIVLRKKTELEAKVNELDERIGHFANIEETLNKSILVAQEAAEDVKRNSQKEAKLIVREAEKNADRIINESLSKSRKIAMEIEELKKQSKVFRTRFQMLIEAQLDLLKNDDWDHLLEYEVDAVFEEKE

SEQ ID NO: 5 - protein, 13 aa, synthetic construct
GAPVPYPDPLEPR

SEQ ID NO: 6 - protein, 118 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 7 - protein, 8 aa, synthetic construct
DYKDDDDK

SEQ ID NO: 8 - DNA, 412 bp, Plutella xylostella
  source 1..412: mol_type = other DNA
gattttctgaacgtccaggacacaagtagaggactggatagtactcgtgtacaggtctatgaggacttctgagtggggactaccgttggcctgaaagaagaatttatggtgagcaatctgactggctaaaccactctccgatgattaaccctagtcaaactctattgtttatcaccgacacggtaatagtatattgaatggacatttagtagataatgtacgattactgctaaaccaccaatattaccaataaggatactcactctagttttcccccaaccagtcgccttaacaggcacattttcgccaatcaggtcatagctcagcggcaccggctggatgagattcgtgaggtggatgggggcggcggcgagcagcatgcccaggtcgttcttgatctcgtcgtgatgca

SEQ ID NO: 9 - protein, 383 aa, Escherichia coli
MFEPMELTNDAVIKVIGVGGGGGNAVEHMVRERIEGVEFFAVNTDAQALRKTAVGQTIQIGSGITKGLGAGANPEVGRNAADEDRDALRAALEGADMVFIAAGMGGGTGTGAAPVVAEVAKDLGILTVAVVTKPFNFEGKKRMAFAEQGITELSKHVDSLITIPNDKLLKVLGRGISLLDAFGAANDVLKGAVQGIAELITRPGLMNVDFADVRTVMSEMGYAMMGSGVASGEDRAEEAAEMAISSPLLEDIDLSGARGVLVNITAGFDLRLDEFETVGNTIRAFASDNATVVIGTSLDPDMNDELRVTVVATGIGMDKRPEITLVTNKQVQQPVMDRYQQHGMAPLTQEQKPVAKVVNDNAPQTAKEPDYLDIPAFLRKQAD

SEQ ID NO: 10 - DNA, 59 bp, synthetic construct
  source 1..59: mol_type = other DNA
ggatgagagggatgaaaaactcaaggcagagataactctgattacacgtcttgagcgat

SEQ ID NO: 11 - DNA, 59 bp, synthetic construct
  source 1..59: mol_type = other DNA
ataaatgggagggtgacttgcctcaatataatccagactactgacatgggaattagcca

SEQ ID NO: 12 - protein, 57 aa, synthetic construct
CTTTGCCATAAAATTCTGCTGGGATTTGATCACTCAGCATTACACGTCTTGAGCGAT

SEQ ID NO: 13 - DNA, 56 bp, synthetic construct
  source 1..56: mol_type = other DNA
ctggcgatgattaatagctaattgagtaaggccaggactgacatgggaattagcca

SEQ ID NO: 14 - DNA, 59 bp, synthetic construct
  source 1..59: mol_type = other DNA
acaagcgtttgaggaagcctttcttctcttcttcaatgaaattacacgtcttgagcgat

SEQ ID NO: 15 - DNA, 59 bp, synthetic construct
  source 1..59: mol_type = other DNA
caaggaatttctatggcacgcattattgttgttacttcggctgacatgggaattagcca

SEQ ID NO: 16 - DNA, 59 bp, synthetic construct
  source 1..59: mol_type = other DNA
ttgagtaaggccaggatgtcaaacacgccaatcgagcttttgtgtaggctggagctgct

SEQ ID NO: 17 - DNA, 60 bp, synthetic construct
  source 1..60: mol_type = other DNA
ctttttgctcaagctggacggtaaccatttccggatcgatcggctgacatgggaattagc

SEQ ID NO: 18 - DNA, 3882 bp, synthetic construct
  source 1..3882: mol_type = other DNA
tcttgtgcaatgtaacatcagagattttgagacacaacgtggctttgttgaataaatcgaacttttgctgagttgaaggatcagcagtgctcttgtgggtccgattcgccagatgataaggaacagagacgggaccaaaacgaaaaaaggcccccctttcgggaggcctcttttctggaatttggtaccgaggcattcagtctgcctgctttcgcaggaacgccggaatgtccagataatccggctctttggctgtctgaggagcattatcattaacaactttggcgactggtttctgctcctgcgtaaggggcgccataccatgttgttgatagcggtccatcacaggctgctgaacctgtttattagtcactaaggtaatttctggtcttttatccatgccgattcccgtggcgacaaccgttacgcgcagttcatcattcatgtcagggtctaaggaggtacctataactacggtcgcgttgtctgaggcaaatgctctaatggtgtttcccacagtctcgaactcatctaaccgaagatcgaaaccggcagtaatatttacaagaacaccgcgggcgccggacaaatcaatatcctctaataacggtgaagatatagccatctcagcagcttcttctgcgcgatcctccccagatgccacgcctgagcccatcatggcatagcccatctctgacatcactgtcctcacatccgcaaaatctacgttcataagcccaggtcgggtgatcaattcggctataccctgaaccgcgcctttcagaacatcattagccgcaccgaacgcatccagcagtgagattcctcgccccaggactttaagcagtttatcatttgggatcgtgatcaatgaatcaacatgtttagaaagctccgtgatgccctgctccgcaaatgccatacgtttcttaccttcaaagttgaacggtttggtaactacggcaacggtcagaatacccagatctttggccacctccgccacaaccggcgccgcgcccgtccccgtgccgccgcccatgcctgcggcgataaaaaccatatctgccccttccaatgcggctctcaaggcgtcgcgatcctcatccgccgcgttgcgtcctacttccggattcgccccagcacccaaacctttggtgatgcctgacccgatctgaatagtttgacctacggcggttttacggagtgcctgagcatctgtgttcactgcaaagaattccacaccctcaatgcgttctctaaccatatgctcaacagcgttgccgccaccaccgccgactcctatgactttaataaccgcatcgttggtaagttccattggctcaaacatctgatcctccttagtatagtgacctcttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccacccgtagaaaagatcaaaagatcttcttgagatcctttttttctgcgcgtaatctgctacttgcaaacaaaaaaaccaccgctaccagcggtggtttgtttgccggatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatttccgctactgaactactcgatgtcagagccagcgtcttgcataaaaaaacccgccgaagcgggtttttacgttattattagaaaaactcatcgagcatcaaatgaaactgcaatttattcatatcaggattatcaataccatatttttgaaaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgaggcggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacagttttattgttcatgatgatatattttta

SEQ ID NO: 19 - DNA, 3449 bp, synthetic construct
  source 1..3449: mol_type = other DNA
ctcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccacccgtagaaaagatcaaaagatcttcttgagatcctttttttctgcgcgtaatctgctacttgcaaacaaaaaaaccaccgctaccagcggtggtttgtttgccggatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatttccgctactgaactactcgatgtcagagccagcgtcttgcataaaaaaacccgccgaagcgggtttttacgttattattagaaaaactcatcgagcatcaaatgaaactgcaatttattcatatcaggattatcaataccatatttttgaaaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgaggcggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagcagtgctcttgtgggtccgattcgccagatgataaggaacagagacgggaccaaaacgaaaaaaggcccccctttcgggaggcctcttttctggaatttggtaccgaggcatttacttatacaactcgtccataccatgagtaattccggcagccgtaacgaactccagcaggaccatgtggtcgcgtttctcgttgggatcttttgataaaacagactgcgtgctaagatagtgattgtcgggtaaaaggaccggcccgtccccaatgggcgtattttgttgatagtggtcagcaagctgaactgacccatcttccacgttgtggcgaatcttaaagttagctttaatcccgttcttctgtttgtcagcggtaatgtagacgttatgtgaattaaagttgtactccagcttgtgcccaagaatatttccatcttctttaaagtcgatccctttcaactcaatgcggttaaccaaagtgtccccttcgaacttcacttcagcgcgagttttataggtcccgtcgtctttgaacgagattgtgcgctcttgcacatagccttccggcattgcgctcttaaagaaatcgtgacgcttcatgtgatctggataacgagagaaacattgaacgccgtaagttaaagtcgtcaccagagtaggccagggcactggcaacttcccagtagtgcagataaacttaagggttaacttcccattagtagcgtccccttccccttccccacgtacgctgaacttatggccgtttacgtcaccgtctaattcgaccaagatgggtactacaccggtgaacaattcctctcccttggacatatttacctcccctatggttcgctgcaacttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccata

SEQ ID NO: 20 - DNA, 4439 bp, synthetic construct
  source 1..4439: mol_type = other DNA
gttgcctggctccccgtcgtgtagataactacgatacgggagggcttaccatctggccccagggctgcaataataccgcgggacccacgctcaccggctccagatttatcagcaataaaccagccagccggaagggccgagcgcagaagtggtcctgcaactttatccgcctccatccagtctattaattgttgccgggaagcgagagtaagaagttcgccagttaagagtttgcgcaacgttgttgccatagctacaggcatcgtggtgtcacgctcgtcgtttggtatggcttcattcagctccggttcccaacgatcaaggcgagttacatgatcacccatgttgtgcaaaaaagcggtaagctccttcggtcctccgatcgttgtcagaagtaagttggccgccgtgttatcactcatggttatggcagcgctgcataattcgcgtactgtcatgccgtccgtaagatgcttttctgtgactggtgagtactcaaccaagtcattctgagaatagtgtatgcggcgaccgagttgctcttgcccggcgtcaatacgggataataccgcgccacagagcagaactttaaaagtgctcatcattgggaaacgttcttcggggcgaaaactctcaagaatcttaccgctgttgaggtccagttcgatgtaacccacacgtgcacccaactgatcttcggcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatagatatttgaatgtatttagcagtgctcttgtgggtccgattcgccagatgataaggaacagagacgggaccaaaacgaaaaaaggcccccctttcgggaggcctcttttctggaatttggtaccgaggcatttaccgtaatttaatcaagcggggatcctgtttccatcttttgaaccaaatattatcttcgtttttaaatttattccacattttctcggcaatagcttcgccgtgggcaaccagcactaacggtaataatcccggattattatctatacgctgttcactttccagaaattctcttatcgtcggtaaggcacgctcactattctgacaatattcagcgtaaagaagattaacagcaataagtcctgttatttcctgcgtggatatttctttagttaattttcgtgccagttcatgtttacctttaagcgaaagaaacataacctgcatacttaataatattggattatcctgcaggtgttgtgagcgtaattcatcgcctaaacgtatagcatcatcaataccggtatgataataggtaatccacagcttagtgatccctgcggctgcgcgcgttgggtccaattttaaacactcgttaatcgtttgtaaggcctcctccaactgaccagccatgaaaagattccagccataataatatttaatatctgcagaaatgggcgaaagtaagttagcttgtttgaatagcaaactcccgacgatgtattctgagtgaatcgtattaattagccccagtaatcctaaagcttgtggattattgtggtccagctctgtcgccttaatcgcatgttctttagctttgatcatagcgttttgtttatcaaaaatccccatttgcgccatgctgaggtagcattctgccagcgcacagtaaggcgcaatgctgtttggcgacatgttaacgcattgagtcagcaatttaagcgcttgctgtaagctatagggggtgtattgatttaactcatgcttaccgcgtaagtaaaccatagtactgtcaatcgaatttagctcgctaatctgctttgtgtcccagcgaagtccgggaatacatctgagcaaaagattcgcaattttgttttgcaagagagaagcgggttggtgttctatcaacttaatgctttcctggtgcagcaggtgataacctttaacccgaactatctcaatctgtacaatattatcattaccatcgggtatcatctgcccggagatataataatcggggcgtaattgatccatgagctcaagaatatccttaacactgcggcagttcttcgtaatggtcaccggcagcacgctcaggccaaagggcgcatactgcgataatcccttcacgatagagtaatgcagactctcggattgaacctgatcctgcatctgaaaaggaagtatcgccaatgtatgagtcgtaggttgcggcgctggcggagacaccactacgaccggacgattaaaccgatagccctgtccgtacagtgtttcaatgtaacgatgctctttatcttccgacagaatacgtcgtaaggcataaatacagcgggtaagagattcttcgttaacttccgcgtcgccccatacctggtccagtaaggtgtttttactcacaatctcgccggcggcttcgagcaggatgaccagaacggcatattcttttggcggaatattgactttcttttctgagcgtagcagggagccgtccatgttgagtatgaaatcatcaaagacgaattttttattcgatacaggaacaggattaaaatgtggcataaaaaaccaacttaggaagccgaagctaattcttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccagatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgcttccgctactgaactactcgatgtcagagccagcgtcttgcataaaaaaacccgccgaagcgggtttttacgttattattaccaatgcttaatcagggaggcacctatctcagcgatctggcgattacgttcgtccata

SEQ ID NO: 21 - DNA, 4439 bp, synthetic construct; identical to SEQ ID NO: 20
  source 1..4439: mol_type = other DNA

SEQ ID NO: 22 - DNA, 4973 bp, synthetic construct
  source 1..4973: mol_type = other DNA
aaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgaggcggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagcagtgctcttgtgggtccgattcgccagatgataaggaacagagacgggaccaaaacgaaaaaaggcccccctttcgggaggcctcttttctggaatttggtaccgaggcatttacttatcatcgtcgtccttataatctccgctggaggaaacggtgacctgcgtaccttgtccccagtaatcagtgccctgcgcagcggtgcgaaagcgtttgcagtaataaacggcagtatcttcgggctttaagctattcatttgaagataaaccgtattctttgcgttgtcgcgggaaatggtgaaacgtcccttcacggagtcagcatagtatgtgtctccgatgctggagatcaacgctaccagctcacgctgtttacccggagcttgacgataccaacccattgtattgatcgagaaggtaatgcctgacgctgcacaggacaggcgtaatgagccaccagcttggaccaaaccgcccccggactccacaagttggacttcagagccaccacgtggctccaagggatccgggtaggggactggcgcaccgctgtttgcgacatccatcattgttgcgcgcagttcagcagttacggtctttgacatgtcgtctacactcactgtaatggttgcttcgccagactgggtcccagtagtaaagacagattggtacacacccgcctcagtttcggtgaactcgcctaatgtaggttttgcttgtgatttggttgccttcagtgtgcgtgttacgatatttcccgcgggcttaaaggtaagctccgtcttaatctggtctttcatgccggtcaccggttgaccttcagcatcacgtaaagacagtaccaatggcttctgctcattgccgtttgccagcatctgaatacgtgattgcccgtctaacgtcaaagcagtgcgatctgcactcatgcctgctccactaatgacaacttcagtttggacgcgtttagacgcatttcctttgttgtcatatgcaatagctgaaattgcgtagtagttatccttaccggcctggtaagcgggcagggtgacctgccactgatttccctgacctgtaatctttccgccagctgccagtaacgatggcgcctcccattgtacgtttttcagcccatgtgtagctttactaacgaccaatcccaagcttaccgtttgcccgcctttaccctcgatgcgttctggcaaggcgatacgaatcacctcagacttacggtactcaagaacgatattattgttacgttctaccaggtcataacgtgatcctgccaacatacgacgctcacgaatcgagtccgtatccaattgcttttcaagtggttcaccgatacgataattaacttccagaccgaaacgcgtgtcattctctcccgactttccctgcttatgacctgcggacaatgtcagtaacggtacaggcgtatagttaacttcagctgtaatggcgtgggggtccttctgacgcttgtctttcccaaacagcccaacttcatcgccatagtattgttcgtacatcaatgaagctcccaactggggccacgctggtaaatatccctcagcacgaatgtcccagccgtttgcggggcgttcttggtaatcctctacatcggggctttttttccaaccggatgcgcgaatatatccgttcgcactcagtttcaagtaatcacgccagtattctgcgcccactccaatacgcgtatgagaacgactaaggtcgtgatcgatgaacgtattcacccccgccatccagtcattctcactaaagtgacgccacccgaacccaatgtttgattgggtgcgatcgtcggtgcgatggattgcgccttgggtgaacaacatgttcgtaggcgtatcgtagattggatacaacatttccagtgatgaatccttcaaggaaaactttttgtcaacattaagcttaacgcgcgccgttccatatttgcccagccattcctggatttcctggttggctttcgcggttgccataccggtaataaaattacgagttgcgtccgaatcgggctggcttgaaagaaaggtgccagcattagccgccagcgaggctacatttttttctacattattgtcggcagtaactgtcgtgttttccatactcaggcgcggttgcacagcgtgctgagcacgcgcagccattactggggtaaaggtgacggctaaaggaaaaagcacctgaacactgatattcgcccatgctacacaacgtgccaacactgagtaacgaaagcgtggttgcttattgtcagttttatagcgtgacatgaaaaacccccttaatgttcttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccagatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgcttccgctactgaactactcgatgtcagagccagcgtcttgcataaaaaaacccgccgaagcgggtttttacgttattattagaaaaactcatcgagcatcaaatgaaactgcaatttattcatatcaggattatcaataccatatttttga

SEQ ID NO: 23 - DNA, 3017 bp, synthetic construct
  source 1..3017: mol_type = other DNA
gtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggattttctgaacgtccaggacacaagtagaggactggatagtactcgtgtacaggtctatgaggacttctgagtggggactaccgttggcctgaaagaagaatttatggtgagcaatctgactggctaaaccactctccgatgattaaccctagtcaaactctattgtttatcaccgacacggtaatagtatattgaatggacatttagtagataatgtacgattactgctaaaccaccaatattaccaataaggatactcactctagttttcccccaaccagtcgccttaacaggcacattttcgccaatcaggtcatagctcagcggcaccggctggatgagattcgtgaggtggatgggggcggcggcgagcagcatgcccaggtcgttcttgatctcgtcgtgatgcacctatagtgagtcgtattaatttcgataagccaggttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcg

SEQ ID NO: 24 - DNA, 3528 bp, synthetic construct
  source 1..3528: mol_type = other DNA
gatacgggagggcttaccatctggccccagggctgcaataataccgcgggacccacgctcaccggctccagatttatcagcaataaaccagccagccggaagggccgagcgcagaagtggtcctgcaactttatccgcctccatccagtctattaattgttgccgggaagcgagagtaagaagttcgccagttaagagtttgcgcaacgttgttgccatagctacaggcatcgtggtgtcacgctcgtcgtttggtatggcttcattcagctccggttcccaacgatcaaggcgagttacatgatcacccatgttgtgcaaaaaagcggtaagctccttcggtcctccgatcgttgtcagaagtaagttggccgccgtgttatcactcatggttatggcagcgctgcataattcgcgtactgtcatgccgtccgtaagatgcttttctgtgactggtgagtactcaaccaagtcattctgagaatagtgtatgcggcgaccgagttgctcttgcccggcgtcaatacgggataataccgcgccacagagcagaactttaaaagtgctcatcattgggaaacgttcttcggggcgaaaactctcaagaatcttaccgctgttgaggtccagttcgatgtaacccacacgtgcacccaactgatcttcggcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatagatatttgaatgtatttagcagtgctcttgtgggtccgattcgccagatgataaggaacagagacgggaccaaaacgaaaaaaggcccccctttcgggaggcctcttttctggaatttggtaccgaggcattcattcagatttccctcctttccatttagaaaaaatacttggttttttcgcagtaacagttaccaattctttcaggagaattttatgcagctcctcctcagaaacatctcgaaacagctcgccacctttggtcagggatataccgccggtttcttctgatacaacaatagtaatcgaatcagtgacttccgatatacccaacgccgcgcggtgacgagtacccaactcttttgataggaacggactatcgctcagcggaagatatgatgctgcggaagcgatttcgttgccctttataataacagccccatcatgaagcggcgtattagggataaatatgttaataaggagctgactagaaatcttggcattcagcggaattccagtctcgatataatcatccattcccgtatcacgcgcgacgctgatcagtgcgccaattcgccgtttagccatatactgtgttgacttttcaattgattcgatcagatgatgttgttcccgttcaattctactgccgtagcgcgtgaaaatattccctcggcccagggtttcaagcgctcttcgcagctccggctggaagataatgataatagccaggaacccccaagtcaacatttgatctgtgatccattccacggtttgcaacccaaaaaaaccgctcaggagtttaaccgcaataataataaagattcctttcagcagttgaaccgccttggtgcctcgaatcagcataattactttgtaaatcacaaaccacacaactagaatgtctacgatattggctaagtagtggaggatgctcatgttggaaaagtccataaaaacctgcttaatcgggtaagagcttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccacccgtagaaaagatcaaaagatcttcttgagatcctttttttctgcgcgtaatctgctacttgcaaacaaaaaaaccaccgctaccagcggtggtttgtttgccggatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatttccgctactgaactactcgatgtcagagccagcgtcttgcataaaaaaacccgccgaagcgggtttttacgttattattaccaatgcttaatcagggaggcacctatctcagcgatctggcgattacgttcgtccatagttgcctggctccccgtcgtgtagataactac

(19) United States
(12) Patent Application Publication
(10) Pub. No.: US 2025/0312478 A1
(43) Pub. Date: Oct. 9, 2025

(54) ACHROMOSOMAL DYNAMIC ACTIVE SYSTEMS

(71) Applicant: Flagship Pioneering Innovations VI, LLC, Cambridge, MA (US)

(72) Inventors: Adam Barclay FISHER, Cambridge, MA (US); Kimberly A. HOMAN, Somerville, MA (US); Troy Patrick HUBBARD, Boston, MA (US); David Barry KOLESKY, Arlington, MA (US); Analise Zaunbrecher REEVES, Cambridge, MA (US); Caitlin Nicole SPAULDING, Boston, MA (US); Hok Hei TAM, Newton, MA (US)

(21) Appl. No.: 19/014,392

(22) Filed: Jan. 9, 2025

Related U.S. Application Data
(63) Continuation of application No. 17/299,377, filed on Jun. 3, 2021, now abandoned.
(60) Provisional application No. 62/777,305, filed on Dec. 10, 2018.

(51) Int. Cl. A61K 47/69; A61K 9/48; C12N 1/08; C12R 1/19; C12R 1/44

(52) U.S. Cl. CPC A61K 47/69; A61K 9/48; C12N 1/08; C12R 2001/19; C12R 2001/44

(57) ABSTRACT

The invention provides isolated achromosomal dynamic active systems (ADAS), including highly active ADAS. These ADAS provided by the invention can be obtained by a variety of means. Various associated methods of making and using these ADAS are provided.

### SEQUENCE LISTING

The instant application contains a Sequence Listing which has been submitted electronically in ASCII format and is hereby incorporated by reference in its entirety. Said ASCII copy, created on Dec. 10, 2019, is named 51296-033WO2_Sequence_Listing_12.10.19_ST25 and is 51,393 bytes in size.

### FIELD OF THE INVENTION

Provided herein are achromosomal dynamic active systems and methods of making and using the same.

### BACKGROUND

A need exists for delivery vectors capable of targeting cells and delivering biological agents, compositions containing such delivery vectors, and associated methods of delivering said vectors to cells, thereby modulating biological systems including animal, plant, and insect cells, tissues, and organisms.

### SUMMARY OF THE INVENTION

The invention provides, inter alia, achromosomal dynamic active systems (ADAS), e.g., ADAS comprising a heterologous cargo, methods of making them, compositions containing them, and associated methods of delivering ADAS and/or cargoes and of modulating biological systems. The invention is based, at least in part, on Applicant's discovery of achromosomal dynamic active systems (ADAS) having, in certain embodiments, enhanced activity (i.e., highly active ADAS). These highly active ADAS have an elevated capacity for work, such as chemical work, protein production, or delivery of a cargo.

In one aspect, the disclosure features a method for manufacturing a composition comprising a plurality of ADAS, the composition being substantially free of viable bacterial cells, the method comprising: (a) making, providing, or obtaining a plurality of parent bacteria having a reduction in the level or activity of a cell division topological specificity factor; (b) exposing the parent bacteria to conditions allowing the formation of a minicell, thereby producing the ADAS; and (c) separating the ADAS from the parent bacteria, thereby producing a composition comprising a plurality of ADAS that is substantially free of viable bacterial cells.

In some embodiments, the cell division topological specificity factor is a polypeptide having an amino acid sequence with at least 40% identity to SEQ ID NO: 1. In some embodiments, the cell division topological specificity factor is a polypeptide having an amino acid sequence with at least 90% identity to SEQ ID NO: 1.

In some embodiments, the cell division topological specificity factor is a minE polypeptide.

In some embodiments, the parent bacteria are E. coli and the minE polypeptide is E. coli minE. In other embodiments, the parent bacteria are Salmonella typhimurium and the minE polypeptide is S. typhimurium minE.

In some embodiments, the parent bacteria have a reduction in the level or activity of a Z-ring inhibition protein. In some embodiments, the Z-ring inhibition protein is a polypeptide having an amino acid sequence with at least 40% identity to SEQ ID NO: 2. In some embodiments, the Z-ring inhibition protein is a polypeptide having an amino acid sequence with at least 90% identity to SEQ ID NO: 2.

In some embodiments, the Z-ring inhibition protein is a polypeptide having an amino acid sequence with at least 40% identity to SEQ ID NO: 3. In some embodiments, the Z-ring inhibition protein is a polypeptide having an amino acid sequence with at least 90% identity to SEQ ID NO: 3.

In some embodiments of the above aspect, the Z-ring inhibition protein is a minC polypeptide or a minD polypeptide.

In some embodiments of the above aspect, the ADAS have a reduction in expression of at least two Z-ring inhibition proteins, e.g., a reduction in expression of a minC polypeptide and a minD polypeptide.

In some embodiments, the ADAS have a reduction in expression of a minC polypeptide, a minD polypeptide, and a minE polypeptide.

In other embodiments of the above aspect, the cell division topological specificity factor is a polypeptide having an amino acid sequence with at least 40% identity to SEQ ID NO: 4, e.g., at least 90% identity to SEQ ID NO: 4. In some embodiments, the cell division topological specificity factor is a DivIVA polypeptide.

In some embodiments, the parent bacteria are Bacillus subtilis and the cell division topological specificity factor is B. subtilis DivIVA.

In some embodiments of the above aspect, the reduction in the level or activity is caused by a loss-of-function mutation. In some embodiments, the loss-of-function mutation is a deletion of the minCDE operon or deleting of DiVIVA.

In some embodiments of the above aspect, the ADAS have an initial ATP concentration of at least 1 mM, 1.2 nM, 1.3 nM, 1.4 mM, 1.5 mM, 1.6 mM, 2 mM, 2.5 mM, 3 mM, 4 mM, 5 mM, 10 mM, 20 mM, 30 mM, or 50 mM.

In some embodiments of the above aspect, the parent bacteria are any one of Escherichia, Acinetobacter, Agrobacterium, Anabaena, Aquifex, Azoarcus, Azotobacter, Bordetella, Bradyrhizobium, Brucella, Buchnera, Burkholderia, Candidatus, Chromobacterium, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylococcus, Neisseria, Nitrosomonas, Nostoc, Photobacterium, Photorhabdus, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Symbiobacterium, or Thermoanaerobacter and the cell division topological specificity factor is the endogenous minE or DivIVA of the parent bacteria.

In some embodiments of the above aspect, the composition of step (c) comprises less than 100 colony-forming units (CFU/mL) of viable bacterial cells, e.g., less than 10 CFU/mL, less than 1 CFU/mL, or less than 0.1 CFU/mL of viable bacterial cells.

In some embodiments of the above aspect, the ADAS comprise a cargo.

In some embodiments of the above aspect, the composition is formulated for delivery to an animal; formulated for delivery to a plant; formulated for delivery to an insect, and/or formulated as a liquid, a solid, an aerosol, a paste, a gel, or a gas composition.

In another aspect, the disclosure features a composition comprising a plurality of highly active achromosomal dynamic active systems (ADAS), wherein the ADAS have an initial ATP concentration of at least 1 mM and wherein the composition is substantially free of viable bacterial cells. In some embodiments, the ADAS have an initial ATP concentration of at least 1.2 nM, 1.3 nM, 1.4 mM, 1.5 mM, 1.6 mM, 2 mM, or 2.5 mM.

In still another aspect, the disclosure features a composition comprising a plurality of highly active ADAS, wherein the ADAS have an initial ATP concentration of at least 3 mM and wherein the composition is substantially free of viable bacterial cells.

In some embodiments, the composition of ADAS have an initial ATP concentration of at least 4 mM, 5 mM, 10 mM, 20 mM, 30 mM, or 50 mM.

In some embodiments of the composition, the ATP concentration of the ADAS is increased by at least 50%, at least 60%, at least 75%, at least 100%, at least 150%, or at least 200% following incubation at 37° C. for 12 hours.

In some embodiments of the composition, the ADAS are derived from parent bacteria having a reduction in a level or activity of a cell division topological specificity factor.

In still other aspect, the invention features a composition comprising a plurality of ADAS, wherein the ADAS do not comprise a cell division topological specificity factor and wherein the composition is substantially free of viable bacterial cells.

In yet another aspect, the invention features a composition comprising a plurality of ADAS, the composition being substantially free of viable bacterial cells, and being produced by a process comprising: (a) making, providing, or obtaining a plurality of parent bacteria having a reduction in the level or activity of a cell division topological specificity factor; (b) exposing the parent bacteria to conditions allowing the formation of a minicell, thereby producing the highly active ADAS; and (c) separating the ADAS from the parent bacteria, thereby producing a composition that is substantially free of viable bacterial cells.

In some embodiments, the cell division topological specificity factor is a polypeptide having an amino acid sequence with at least 40% identity to SEQ ID NO: 1.

In some embodiments, the cell division topological specificity factor is a polypeptide having an amino acid sequence with at least 90% identity to SEQ ID NO: 1.

In some embodiments, the cell division topological specificity factor is a minE polypeptide.

In some embodiments, the parent bacteria are E. coli and the minE polypeptide is E. coli minE.

In some embodiments, the parent bacteria are Salmonella typhimurium and the minE polypeptide is S. typhimurium minE.

In some embodiments, the parent bacteria are Escherichia, Acinetobacter, Agrobacterium, Anabaena, Aquifex, Azoarcus, Azotobacter, Bordetella, Bradyrhizobium, Brucella, Buchnera, Burkholderia, Candidatus, Chromobacterium, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylococcus, Neisseria, Nitrosomonas, Nostoc, Photobacterium, Photorhabdus, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Symbiobacterium, or Thermoanaerobacter and the cell division topological specificity factor is the endogenous minE or DivIVA of the parent bacteria.

In some embodiments, the ADAS have a reduction in a level of a Z-ring inhibition protein.

In some embodiments, the Z-ring inhibition protein is a polypeptide having an amino acid sequence with at least 40% identity to SEQ ID NO: 2.

In some embodiments, the Z-ring inhibition protein is a polypeptide having an amino acid sequence with at least 40% identity to SEQ ID NO: 3.

In some embodiments, the Z-ring inhibition protein is a minC polypeptide.

In some embodiments, the Z-ring inhibition protein is a minD polypeptide.

In some embodiments, the ADAS have a reduction in expression of at least two Z-ring inhibition proteins.

In some embodiments, the ADAS have a reduction in expression of a minC polypeptide and a minD polypeptide.

In some embodiments, the ADAS have a reduction in expression of a minC polypeptide, a minD polypeptide, and a minE polypeptide.

In some embodiments, the cell division topological specificity factor is a polypeptide having an amino acid sequence with at least 40% identity to SEQ ID NO: 4.

In some embodiments, the cell division topological specificity factor is a polypeptide having an amino acid sequence with at least 90% identity to SEQ ID NO: 4.

In some embodiments, the cell division topological specificity factor is a DivIVA polypeptide.

In some embodiments, the parent bacteria are Bacillus subtilis and the cell division topological specificity factor is B. subtilis DivIVA.

In some embodiments, the reduction in the level or activity is caused by a loss-of-function mutation.

In some embodiments, the loss-of-function mutation is a gene deletion.

In some embodiments, the loss-of-function mutation is an inducible loss-of-function mutation and wherein loss of function is induced by exposing the parent cell to an inducing condition.

In some embodiments, the inducible loss-of-function mutation is a temperature-sensitive mutation and wherein the inducing condition is a temperature condition.

In some embodiments, the parent cell has a deletion of the minCDE operon.

In some embodiments, the ADAS comprise a functional transcription system and a functional translation system.

In some embodiments, the ADAS produce a heterologous protein.

In some embodiments, the ADAS comprise a plasmid, the plasmid comprising an inducible promoter and a nucleotide sequence encoding the heterologous protein, and wherein contacting the ADAS with an inducer of the inducible promoter under appropriate conditions results in production of the heterologous protein.

In some embodiments, the production of the heterologous protein is increased by at least 1.6-fold in an ADAS that has been contacted with the inducer relative to an ADAS that has not been contacted with the inducer.

In some embodiments, the rate of production of the heterologous protein reaches a target level within 3 hours of the contacting of the ADAS with the inducer.

In some embodiments, the heterologous protein is produced at a rate of at least 0.1 femtograms per hour per ADAS.

In some embodiments, the heterologous protein is produced for a duration of at least 8 hours.

In some embodiments, the composition comprises less than 100 colony-forming units (CFU/mL) of viable bacterial cells.

In some embodiments, the composition comprises less than 10 CFU/mL, less than 1 CFU/mL, or less than 0.1 CFU/mL of viable bacterial cells.

In some embodiments, the ADAS comprise a cargo.

In some embodiments, the cargo is a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP).

In some embodiments, the cargo is encapsulated by the ADAS.

In some embodiments, the cargo is attached to the surface of the ADAS.

In some embodiments, the nucleic acid is a DNA, an RNA, or a plasmid.

In some embodiments, the nucleic acid encodes a protein.

In some embodiments, the enzyme alters a substrate to produce a target product.

In some embodiments, the substrate is present in the ADAS and wherein the target product is produced in the ADAS.

In some embodiments, the substrate is present in a target cell or environment to which the ADAS is delivered.

In some embodiments, the ADAS comprises a heterologous bacterial secretion system.

In some embodiments, the heterologous bacterial secretion system is a type 3 secretion system (T3SS).

In some embodiments, the cargo comprises a moiety that directs export by the bacterial secretion system.

In some embodiments, the ADAS comprises a targeting moiety.

In some embodiments, the targeting moiety is a nanobody, a carbohydrate binding protein, or a tumor-targeting peptide.

In some embodiments, the ADAS have a reduced protease level or activity relative to an ADAS produced from a wild-type parent bacterium.

In some embodiments, the ADAS are produced from parent bacteria that have been modified to reduce or eliminate expression of at least one protease.

In some embodiments, the ADAS have a reduced RNase level or activity relative to an ADAS produced from a wild-type patent bacterium.

In some embodiments, the ADAS are produced from parent bacteria that have been modified to reduce or eliminate expression of at least one RNase.

In some embodiments, the RNase is an endoribonuclease or an exoribonuclease.

In some embodiments, the ADAS has been modified to have reduced lipopolysaccharide (LPS).

In some embodiments, the ADAS are produced from parent bacteria that have been modified to have reduced LPS.

In some embodiments, the modification is a mutation in Lipid A biosynthesis myristoyltransferase (msbB).

In some embodiments, the ADAS are derived from parental bacteria that are pathogens of mammals or from parental bacteria that are commensal to mammals.

In some embodiments, the mammalian commensal bacteria are Staphylococcus, Bifidobacterium, Micrococcus, Lactobacillus, or Actinomyces species or the mammalian pathogenic bacteria are enterohemorrhagic Escherichia coli (EHEC), Salmonella typhimurium, Shigella flexneri, Yersinia enterolitica, or Helicobacter pylori.

In some embodiments, ADAS are derived from parent bacteria that are plant pathogens or from parent bacteria that are commensal to plants.

In some embodiments, plant commensal bacteria are Bacillus subtilis or Psuedomonas putida or the plant pathogenic bacteria are Xanthomonas species or Pseudomonas syringae.

In some embodiments, ADAS are derived from auxotrophic parent bacteria.

In some embodiments, the ADAS are lyophilized and reconstituted, and wherein the reconstituted ADAS have an ATP concentration that is at least 95% of the ATP concentration of an ADAS that has not been lyophilized.

In some embodiments, the reconstituted ADAS have an ATP concentration that is at least equal to the ATP concentration of an ADAS that has not been lyophilized.

In some embodiments, the composition is formulated for delivery to an animal.

In some embodiments, the composition is formulated for intraperitoneal, intravenous, intramuscular, oral, topical, aerosolized, or nebulized administration.

In some embodiments, the composition is formulated for delivery to a plant.

In some embodiments, the composition is formulated for delivery to an insect.

In some embodiments, the composition is formulated as a liquid, a solid, an aerosol, a paste, a gel, or a gas composition.

In another aspect, the invention features a method for delivering a highly active ADAS to a target cell, the method comprising: (a) providing a composition comprising a plurality of highly active ADAS, wherein the ADAS have an initial ATP concentration of at least 1.25 mM and wherein the composition is substantially free of viable bacterial cells; and (b) contacting the target cell with the composition of step (a).

In still another aspect, the invention features a method for delivering an ADAS to a target cell, the method comprising: (a) providing a composition comprising a plurality of ADAS, wherein the ADAS are derived from a plurality parent bacteria having a reduction in the level or activity of a cell division topological specificity factor and wherein the composition is substantially free of viable bacterial cells; and (b) contacting the target cell with the composition of step (a).

In some embodiments regarding the method of delivering such a plurality of ADAS, the target cell is an animal cell, a plant cell or an insect cell.

In yet another aspect, the invention features a method for delivering a cargo to a target cell, the method comprising: (a) providing a composition comprising a plurality of highly active achromosomal dynamic active systems (ADAS), wherein the ADAS have an initial ATP concentration of at least 1.25 mM, the ADAS comprise a cargo, and the composition is substantially free of viable bacterial cells; and (b) contacting the target cell with the composition of step (a).

In still another aspect, the invention features a method for delivering a cargo to a target cell, the method comprising: (a) providing a composition comprising a plurality of ADAS, wherein the ADAS are derived from a plurality parent bacteria having a reduction in the level or activity of a cell division topological specificity factor, the ADAS comprise a cargo, and the composition is substantially free of viable bacterial cells; and (b) contacting the target cell with the composition of step (a).

In embodiments regarding delivering a cargo, the target cell is an animal cell, a plant cell, or an insect cell.

In another aspect, the invention features a method of modulating a state of an animal cell, the method comprising: (a) providing a composition comprising a plurality of highly active achromosomal dynamic active systems (ADAS), wherein the ADAS have an initial ATP concentration of at least 1.25 mM and wherein the composition is substantially free of viable bacterial cells; and (b) contacting the animal cell with the composition of step (a), whereby a state of the animal cell is modulated.

In still another aspect, the method features a method of modulating a state of a plant cell, the method comprising: (a) providing a composition comprising a plurality of highly active achromosomal dynamic active systems (ADAS), wherein the ADAS have an initial ATP concentration of at least 1.25 mM and wherein the composition is substantially free of viable bacterial cells; and (b) contacting the plant cell with the composition of step (a), whereby a state of the plant cell is modulated.

In another aspect, the invention features a method of modulating a state of an insect cell, the method comprising: (a) providing a composition comprising a plurality of highly active achromosomal dynamic active systems (ADAS), wherein the ADAS have an initial ATP concentration of at least 1.25 mM and wherein the composition is substantially free of viable bacterial cells; and (b) contacting the insect cell with the composition of step (a), whereby a state of the insect cell is modulated.

In another aspect, the invention features a method of modulating a state of an animal cell, the method comprising: (a) providing a composition comprising a plurality of ADAS, wherein the ADAS are derived from a plurality of parent bacteria having a reduction in the level or activity of a cell division topological specificity factor and wherein the composition is substantially free of viable bacterial cells; and (b) contacting the animal cell with the composition of step (a), whereby a state of the animal cell is modulated.

In another aspect, the invention features a method of modulating a state of a plant cell, the method comprising: (a) providing a composition comprising a plurality of ADAS, wherein the ADAS are derived from a plurality of parent bacteria having a reduction in the level or activity of a cell division topological specificity factor and wherein the composition is substantially free of viable bacterial cells; and (b) contacting the plant cell with the composition of step (a), whereby a state of the plant cell is modulated.

In another aspect, the invention features a method of modulating a state of an insect cell, the method comprising: (a) providing a composition comprising a plurality of ADAS, wherein the ADAS are derived from a plurality of parent bacteria having a reduction in the level or activity of a cell division topological specificity factor and wherein the composition is substantially free of viable bacterial cells; and (b) contacting the insect cell with the composition of step (a), whereby a state of the insect cell is modulated.

In still another aspect, the invention features a method of treating an animal in need thereof, the method comprising: (a) providing a composition comprising a plurality of highly active achromosomal dynamic active systems (ADAS), wherein the ADAS have an initial ATP concentration of at least 1.25 mM and wherein the composition is substantially free of viable bacterial cells; and (b) contacting the animal with an effective amount of the composition of step (a), thereby treating the animal.

In still another aspect, the invention features a method of treating an animal in need thereof, the method comprising: (a) providing a composition comprising a plurality of ADAS, wherein the ADAS are derived from a plurality of parent bacteria having a reduction in the level or activity of a cell division topological specificity factor and wherein the composition is substantially free of viable bacterial cells; and (b) contacting the animal with an effective amount of the composition of step (a), thereby treating the animal.

In some embodiments of treating the animal, the animal has a cancer.

In some embodiments, the ADAS carries a chemotherapy cargo (for example, an immunotherapy cargo).

In yet another aspect, the invention features a method of treating a plant in need thereof, the method comprising: (a) providing a composition comprising a plurality of highly active achromosomal dynamic active systems (ADAS), wherein the ADAS have an initial ATP concentration of at least 1.25 mM and wherein the composition is substantially free of viable bacterial cells; and (b) contacting the plant or a pest thereof with an effective amount of the composition of step (a), thereby treating the plant.

In yet another aspect, the invention features a method of treating a plant in need thereof, the method comprising: (a) providing a composition comprising a plurality of ADAS, wherein the ADAS are derived from a plurality of parent bacteria having a reduction in the level or activity of a cell division topological specificity factor and wherein the composition is substantially free of viable bacterial cells; and (b) contacting the plant or a pest thereof with an effective amount of the composition of step (a), thereby treating the plant.

In another aspect, the invention features a composition comprising a plurality of ADAS, wherein the ADAS comprise an enzyme and wherein the enzyme alters a substrate to produce a target product.

In some embodiments of this composition, the substrate is present in the ADAS and wherein the target product is produced in the ADAS.

In some embodiments, the substrate is present in a target cell or environment to which the ADAS is delivered.

In some embodiments, the enzyme is diadenylate cyclase A, the substrate is ATP, and the target product is cyclic-di-AMP.

Other features and advantages of the invention will be apparent from the following Detailed Description and the Claims.

### BRIEF DESCRIPTION OF THE DRAWINGS

FIG. 1A is a set of scanning electron micrographs showing E. coli parent bacterial cells and achromosomal dynamic active cells (ADAS) derived therefrom. The left column shows bacterial cells of the E. coli strain MACH009, which has not been modified to produce ADAS. The center column shows bacterial cells of the E. coli strain MACH060 (AminCDE), which has been modified to produce ADAS. ADAS and parent cells undergoing abnormal septation events are visible. The right column shows ADAS purified from the MACH060 (AminCDE) culture.

FIG. 1B is a set of photomicrographs showing ADAS derived from MACH060 (AminCDE). The left panel is a phase contrast image showing the location of particles. The center panel is an immunofluorescence image showing the localization of the DNA stain DAPI. The right panel is an immunofluorescence image showing the localization of a stain for lipopolysaccharide (LPS). White arrows indicate ADAS, which are identified by the presence of the LPS stain and the absence of the DAPI stain. Scale bars are 10 μm.

FIG. 1C is a graph showing the concentration (in particles/mL) of particles of a given diameter in a purified population of ADAS from the ADAS-producing E. coli strain MACH124 (AminCDE), as measured using a Spectradyne® nCS1 Nanoparticle Analyzer.

FIG. 1D is a bar graph showing the aggregate volume of particles between 200 and 2000 nm in diameter in the purified ADAS preparation.

FIG. 1E is a bar graph showing the concentration in colony-forming units (CFU) per mL of viable bacterial cells present in a MACH124 (AminCDE) culture and in an ADAS preparation purified from the culture.

FIG. 1F is an image of an immunoblot for Dnak and GroEL in cultures of the ADAS-producing E. coli strains MACH060 and MACH200 (both AminCDE) and in ADAS preparations purified from each culture. 10 ng of purified Dnak and GroEL are provided as controls (Recomb. Prot.).

FIG. 2A is a bar graph showing the average concentration of ATP in mMol in a purified population of ADAS from the E. coli strain MACH060 (AminCDE), as measured using the BacTiter-Glo™ Microbial Cell Viability Assay. The ATP concentration generated by parent bacteria was subtracted as background.

FIG. 2B is a bar graph showing the luminescence in an aliquot of a purified population of ADAS from the E. coli strain MACH060 (AminCDE) at 0 hours or after 2 hours of shaking incubation at 37° C. in the presence of excess nutrients, as measured using the BacTiter-Glo™ Microbial Cell Viability Assay.

FIG. 2C is a graph showing GFP fluorescence (au) over time in MACH124 (AminCDE) culture and in ADAS preparations purified from the culture that have been incubated in the presence or absence of the inducer anhydrotetracycline (100 ng/ml), which activates expression of GFP. All samples were grown in the presence of antibiotic to prevent any bacterial growth. The concentration of parent bacteria was equivalent to the number of CFU detected in the ADAS preparation (100 CFU/mL).

FIG. 2D is a graph showing the GFP fluorescence data of FIG. 2C normalized to the number of ADAS in the preparation, as measured using a Spectradyne® nCS1 Nanoparticle Analyzer. Data is shown in the graph as fold induction per 108 ADAS particles.

FIG. 2E is a graph showing the fold change in GFP fluorescence between the induced and uninduced ADAS preparations of FIG. 2C. Fold change was calculated by dividing the average value of the induced replicate by the average value of the uninduced replicate.

FIG. 2F is a set of images of an immunoblot for GFP in 250 μL aliquots from a purified ADAS preparation purified from MACH124 (AminCDE) and grown in the presence of the inducer anhydrotetracycline for 0, 2, 4, 17, or 24 hours (upper panel). Each lane corresponds to approximately 5.2×10′ ADAS as determined by nCS1 analysis. GFP intensity was quantified by densitometry, normalized to intensity of the loading control GroEL, and converted to mass of GFP (in ng) by comparison against a molecular standard of purified recombinant GFP processed on the same blot (lower panel).

FIG. 3A is a graph showing GFP fluorescence (au) per 108 ADAS particles over time in ADAS preparations purified from MACH124 (AminCDE), MACH556 (AminC), and MACH557 (AminD) E. coli parent bacterial strains. ADAS were incubated in the presence or absence of the inducer anhydrotetracycline (100 ng/mL), which activates expression of GFP. GFP fluorescence was calculated by subtracting the average value of the uninduced replicates from each induced replicate. All samples were grown in the presence of antibiotic to prevent any bacterial growth.

FIG. 3B is a graph showing the fold change in GFP fluorescence between the induced and uninduced ADAS preparations of FIG. 3A. Fold change was calculated by dividing the average value of the induced replicate by the average value of the uninduced replicate.

FIG. 3C is an image of an immunoblot for GFP in ADAS purified from MACH124 (AminCDE), MACH556 (AminC), and MACH557 (AminD) and grown in the presence (+) or absence (−) of the inducer anhydrotetracycline.

FIG. 3D is a bar graph showing background correction for the immunoblot of FIG. 3C.

FIG. 3E is a bar graph showing normalized GFP density for the immunoblot of FIG. 3C. GFP intensity was quantified by densitometry, and normalized to intensity of the loading control GroEL.

FIG. 3F is a bar graph showing total GFP production in fluorescence units (au) in ADAS purified from MACH124 (AminCDE), MACH556 (AminC), and MACH557 (AminD) as measured using a BioTek® microplate reader. ** indicates a p value of 0.0021. *** indicates a p value of 0.0006.

FIG. 3G is a bar graph showing GFP production per hour in ADAS purified from MACH124 (AminCDE), MACH556 (AminC), and MACH557 (AminD).

FIG. 3H is a bar graph showing the percent of GFP production in ADAS purified from MACH556 (AminC) and MACH557 (AminD) relative to ADAS produced from MACH124 (AminCDE). The protein production level of MACH124 was set to 100%, and relative protein production for MACH556 and MACH557 were normalized to MACH124.

FIG. 4 is a bar graph showing fluorescence of a DyLight™ 550 fluorophore for ADAS purified from MACH060 and MACH284 normalized to optical density of the culture (OD600). MACH284 expresses a Neae-NB2 (nanobody) fusion protein detectable by an antibody conjugated to the DyLight™ 550 fluorophore.

FIG. 5A is a bar graph showing the luminescence readout of the THP1-Dual™ system in THP1-Dual™ monocytes contacted with ADAS purified from MACH060, uninduced ADAS purified from MACH198, and ADAS purified from MACH198 that have been induced with anhydrotetracycline to produce the enzyme deadenylate cyclase A (DacA).

FIG. 5B is a set of micrographs showing the phenotype of THP1-Dual™ monocytes contacted with ADAS purified from MACH060, uninduced ADAS purified from MACH198, and ADAS purified from MACH198 that have been induced with anhydrotetracycline to produce the enzyme DacA.

FIG. 5C is a bar graph showing the concentration of interferon beta (IFN-B1) in pg/mL secreted into culture by THP1-Dual™ monocytes contacted with ADAS purified from MACH060, uninduced ADAS purified from MACH198, and ADAS purified from MACH198 that have been induced with anhydrotetracycline to produce the enzyme DacA, as measured using an ELISA assay.

FIG. 6 is a set of photomicrographs showing fluorescence of DyLight™ 800 NHS Ester-labeled ADAS derived from MACH301 in European Corn Borer (ECB) larvae. Panels A and B show fluorescence of DyLight™ 800. Panels C and D show larval autofluorescence at 700 nm. Panels A and C show control larvae fed with PBS, and panels B and D show larvae fed with the DyLight™ 800 NHS Ester-labeled ADAS.

FIG. 7A is a graph showing GFP fluorescence (au) per 108 ADAS particles over time in ADAS preparations purified from a MACH124 (AminCDE) E. coli parent bacterial strain, wherein the ADAS preparation was stored at 4° C. for 0 days of 3 days. ADAS were incubated in the presence or absence of the inducer anhydrotetracycline (100 ng/ml), which activates expression of GFP. GFP fluorescence was calculated by subtracting the average value of the uninduced replicates from each induced replicate. All samples were grown in the presence of antibiotic to prevent any bacterial growth.

FIG. 7B is a graph showing GFP fluorescence (au) per 108 ADAS particles over time in ADAS preparations purified from a MACH556 (AminC) E. coli parent bacterial strain, wherein the ADAS preparation was stored at 4° C. for 0 days of 3 days. ADAS were incubated in the presence or absence of the inducer anhydrotetracycline (100 ng/ml), which activates expression of GFP. GFP fluorescence was calculated by subtracting the average value of the uninduced replicates from each induced replicate. All samples were grown in the presence of antibiotic to prevent any bacterial growth.

FIG. 7C is a graph showing GFP fluorescence (au) per 108 ADAS particles over time in ADAS preparations purified from a MACH557 (AminD) E. coli parent bacterial strain, wherein the ADAS preparation was stored at 4° C. for 0 days of 3 days. ADAS were incubated in the presence or absence of the inducer anhydrotetracycline (100 ng/ml), which activates expression of GFP. GFP fluorescence was calculated by subtracting the average value of the uninduced replicates from each induced replicate. All samples were grown in the presence of antibiotic to prevent any bacterial growth.

FIG. 7D is a graph showing the fold change in GFP fluorescence between the induced and uninduced ADAS preparations of FIG. 7A. Fold change was calculated by dividing the average value of the induced replicate by the average value of the uninduced replicate.

FIG. 7E is a graph showing the fold change in GFP fluorescence between the induced and uninduced ADAS preparations of FIG. 7B. Fold change was calculated by dividing the average value of the induced replicate by the average value of the uninduced replicate.

FIG. 7F is a graph showing the fold change in GFP fluorescence between the induced and uninduced ADAS preparations of FIG. 7C. Fold change was calculated by dividing the average value of the induced replicate by the average value of the uninduced replicate.

FIG. 7G is a graph showing GFP fluorescence (au) per 108 ADAS particles over time in ADAS preparations purified from a MACH124 (AminCDE) E. coli parent bacterial strain, wherein the ADAS preparation was lyophilized, stored for 6 weeks, and rehydrated. ADAS were incubated in the presence or absence of the inducer anhydrotetracycline (100 ng/ml), which activates expression of GFP. GFP fluorescence was calculated by subtracting the average value of the uninduced replicates from each induced replicate. All samples were grown in the presence of antibiotic to prevent any bacterial growth.

FIG. 7H is a graph showing the fold change in GFP fluorescence between the induced and uninduced ADAS preparations of FIG. 7G. Fold change was calculated by dividing the average value of the induced replicate by the average value of the uninduced replicate.

FIG. 8A is a bar graph showing the number of parent bacterial cells (in CFU/mL) in ADAS preparations purified from the control E. coli parent bacterial cell line MACH060 or the auxotrophic E. coli parent bacterial cell line MACH002 stored in histidine-free and methionine-free media.

FIG. 8B is a bar graph showing the number of parent bacterial cells (in CFU/mL) in ADAS preparations purified from the control E. coli parent bacterial cell line MACH178 or the auxotrophic E. coli parent bacterial cell line MACH151 stored in leucine-free media.

### DETAILED DESCRIPTION OF THE INVENTION

### I. Definitions

As used herein, the term “achromosomal dynamic system” or “ADAS” refers to a genome-free, non-replicating, enclosed membrane system comprising at least one membrane and having an interior volume suitable for containing a cargo (e.g., one or more of a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)). In some embodiments, ADAS are minicells or modified minicells derived from a parent bacterial cell (e.g., a gram-negative or a gram-positive bacterial cell). ADAS may be derived from parent bacteria using any suitable method, e.g., genetic manipulation of the parent cell or exposure to a culture or condition that increases the likelihood of formation of bacterial minicells. Exemplary methods for making ADAS are those that disrupt the cell division machinery of the parent cell. In some embodiments, ADAS may comprise one or more endogenous or heterologous features of the parent cell surface, e.g., cell walls, cell wall modifications, flagella, or pilli, and/or one or more endogenous or heterologous features of the interior volume of the parent cell, e.g., nucleic acids, plasmids, proteins, small molecules, transcription machinery, or translation machinery. In other embodiments, ADAS may lack one or more features of the parent cell. In still other embodiments, ADAS may be loaded or otherwise modified with a feature not comprised by the parent cell.

As used herein, the term “highly active ADAS” refers to an ADAS having high work potential, e.g. an ADAS having the capability to do a significant amount of useful work. Work may be metabolic work, including chemical synthesis (e.g., of proteins, nucleic acids, lipids, carbohydrates, polymers, or small molecules), chemical modification (e.g., of proteins, nucleic acids, lipids, carbohydrates, polymers or small molecules), or transport (e.g., import, export, or secretion) under suitable conditions. In certain embodiments, highly active ADAS begin with a large pool of energy, e.g., energy in the form of ATP. In other embodiments, ADAS have the capacity to take up or generate energy/ATP from another source. Highly active ADAS may be identified, e.g., by having increased ATP concentration, increased ability to generate ATP, increased ability to produce a protein, increased rate or amount of production of a protein, and/or increased responsiveness to a biological signal, e.g., induction of a promoter.

As used herein, the term “parent bacterial cell” refers to a cell (e.g., a gram-negative or a gram-positive bacterial cell) from which an ADAS is derived. Parent bacterial cells are typically viable bacterial cells. The term “viable bacterial cell” refers to a bacterial cell that contains a genome and is capable of cell division. Preferred parent bacterial cells are derived from any of the strains in Table 4.

An ADAS composition or preparation that is “substantially free of” parent bacterial cells and/or viable bacterial cells is defined herein as a composition having no more than 500, e.g., 400, 300, 200, 150, 100 or fewer colony-forming units (CFU) per mL. An ADAS composition that is substantially free of parent bacterial cells or viable bacterial cells may include fewer than 50, fewer than 25, fewer than 10, fewer than 5, fewer than 1, fewer than 0.1, or fewer than 0.001 CFU/mL. including no bacterial cells.

The term “cell division topological specificity factor” refers to a component of the cell division machinery in a bacterial species that is involved in the determination of the site of the septum and functions by restricting the location of other components of the cell division machinery, e.g., restricting the location of one or more Z-ring inhibition proteins. Exemplary cell division topological specificity factors include minE, which was first discovered in E. coli and has since been identified in a broad range of gram-negative bacterial species and gram-positive bacterial species (Rothfield et al., Nature Reviews Microbiology, 3:959-968, 2005). minE functions by restricting the Z-ring inhibition proteins minC and minD to the poles of the cell. A second exemplary cell division topological specificity factor is DivIVA, which was first discovered in Bacillus subtilis (Rothfield et al., Nature Reviews Microbiology, 3:959-968, 2005).

The term “Z-ring inhibition protein” refers to a component of the cell division machinery in a bacterial species that is involved in the determination of the site of the septum and functions by inhibiting the formation of a stable FtsZ ring or anchoring such a component to a membrane. The localization of Z-ring inhibition proteins may be modulated by cell division topological specificity factors, e.g., MinE and DivIVA. Exemplary Z-ring inhibition proteins include minC and minD, which were first discovered in E. coli and have since been identified in a broad range of gram-negative bacterial species and gram-positive bacterial species (Rothfield et al., Nature Reviews Microbiology, 3:959-968, 2005). In E. coli and in other species, minC, minD, and minE occur at the same genetic locus, which may be referred to as the “min operon”, the minCDE operon, or the min or minCDE genetic locus.

As used herein, the term “reduction in the level or activity of a cell topological specificity factor,” refers to an overall reduction of any of 5%, 10%, 20%, 30%, 40%, 50%, 60%, 70%, 80%, 90%, 95%, 96%, 97%, 98%, 99%, or greater, in the level or activity of the cell topological specificity factor (e.g., protein or nucleic acid (e.g., gene or mRNA)), detected by standard methods, as compared to the level in a reference sample (for example, an ADAS produced from a wild-type cell or a cell having a wild-type minCDE operon or wild-type divIVA gene), a reference cell (for example, a wild-type cell or a cell having a wild-type minC, minD, minE, divIVA, or minCDE gene or operon), a control sample, or a control cell. In some embodiments, a reduced level or activity refers to a decrease in the level or activity in the sample which is at least about 0.9×, 0.8×, 0.7×, 0.6×, 0.5×, 0.4×, 0.3×, 0.2×, 0.1×, 0.05×, or 0.01× the level or activity of the cell topological specificity factor in a reference sample, reference cell, control sample, or control cell.

As used herein the term “percent identity” refers to percent (%) sequence identity with respect to a reference polynucleotide or polypeptide sequence following alignment by standard techniques. Alignment for purposes of determining percent nucleic acid or amino acid sequence identity can be achieved in various ways that are within the capabilities of one of skill in the art, for example, using publicly available computer software such as BLAST, BLAST-2, PSI-BLAST, or Megalign software. Those skilled in the art can determine appropriate parameters for aligning sequences, including any algorithms needed to achieve maximal alignment over the full length of the sequences being compared.

For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 Multiplied by (the Fraction X/Y)
where X is the number of nucleotides or amino acids scored as identical matches by a sequence alignment program (e.g., BLAST) in that program's alignment of A and B, and where Y is the total number of nucleotides or amino acids in B. In some embodiments, sequence identity, for example, in homologues of MinE or DivIVA proteins will have at least about 40%, 50%, 60%, 70%, 80%, 85%, 90%, or even 95% or greater amino acid or nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% or greater amino acid sequence or nucleic acid identity, to a native sequence MinE (or minE) or DivIVA (or divIVA) sequence as disclosed herein.

The phrases “modulating a state of a cell” as used herein, refers to an observable change in the state (e.g., the transcriptome, proteome, epigenome, biological effect, or health or disease state) of the cell (e.g., an animal, plant, or insect cell) as measured using techniques and methods known in the art for such a measurement, e.g., methods to measure the level or expression of a protein, a transcript, an epigenetic mark, or to measure the increase or reduction of activity of a biological pathway. Modulating the state of the cell may result in a change of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In some embodiments, modulating the state of the cell involves increasing a parameter (e.g., the level or expression of a protein, a transcript, or activity of a biological pathway) of the cell. Increasing the state of the cell may result in an increase of the parameter by at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In other embodiments, modulating the state of involves decreasing a parameter (e.g., the level or expression of a protein, a transcript, or activity of a biological pathway) of the cell. Decreasing the state of the cell may result in a decrease of the parameter by at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration).

As used herein, the term “heterologous” means not native to a cell or composition in its naturally-occurring state. In some embodiments “heterologous” refers to a molecule; for example, a cargo or payload (e.g., a polypeptide, a nucleic acid such as a protein-encoding RNA or tRNA, or small molecules) or a structure (e.g., a plasmid or a gene-editing system) that is not found naturally in an ADAS or the parent bacteria from which it is produced (e.g., a gram-negative or gram positive bacterial cell).

### II. Compositions

### A. ADAS and Highly Active ADAS

The invention is based, at least in part, on Applicant's discovery of achromosomal dynamic active systems (ADAS), including highly active ADAS, which are able to provide a wide array of functions in a large number of environments. An “ADAS” is a genome-free, non-replicating, enclosed membrane system comprising at least one membrane (in some embodiments, two membranes, where the two membranes are non-intersecting) and having an interior volume suitable for containing a cargo (e.g., a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)).

In some embodiments, ADAS are minicells or modified minicells derived from a parent bacterial cell (e.g., a gram-negative or a gram-positive bacterial cell). ADAS may be derived from parent bacteria using any suitable method, e.g., genetic manipulation of the parent cell or exposure to a culture or condition that increases the likelihood of formation of bacterial minicells.

In some embodiments, an ADAS has a major axis cross section between about 100 nm-500 μm (e.g., in certain embodiments, about: 100-600 nm, such as 100-400 nm; or between about 0.5-10 μm, and 10-500 μm). In certain embodiments, an ADAS has a minor axis cross section between about: 0.001, 0.01, 0.1, 1, 2, 3, 4, 5, 6, 7, 8, 9, 10, 11, 12, 13, 14, 15, 20, 30, 40, 50, 60, 70, 80, 90, up to 100% of the major axis. In certain embodiments, an ADAS has an interior volume of between about: 0.001-1 μm³, 0.3-5 μm³, 5-4000 μm³, or 4000-50×107 μm³.

In some embodiments, the invention provides highly active ADAS. A “highly active” ADAS is an ADAS with high work potential, e.g. an ADAS having the capability to do a significant amount of useful work. Work may be defined as, e.g., metabolic work, including chemical synthesis (e.g., synthesis of proteins, nucleic acids, lipids, carbohydrates, polymers, or small molecules), chemical modification (e.g., modification of proteins, nucleic acids, lipids, carbohydrates, polymers or small molecules), or transport (e.g., import, export, or secretion) under suitable conditions. In some embodiments, highly active ADAS begin with a large pool of energy, e.g., energy in the form of adenosine triphosphate (ATP). In other embodiments, ADAS have the capacity to take up or generate energy (e.g., ATP) from another source.

The term “ADAS provided by the invention” encompasses all embodiments of ADAS described herein, including, in particular embodiments, highly active ADAS, the set of which can be referenced as “highly active ADAS provided by the invention”, which is a subset of the ADAS provided by the invention.

In one aspect, the invention provides a composition comprising a plurality of highly active achromosomal dynamic active systems (ADAS), wherein the ADAS have an initial ATP concentration of at least 1 mM and wherein the composition is substantially free of viable bacterial cells.

In another aspect, the invention provides a composition comprising a plurality of highly active achromosomal dynamic active systems (ADAS), wherein the ADAS have an initial ATP concentration of at least 3 mM and wherein the composition is substantially free of viable bacterial cells.

In some embodiments, a highly active ADAS has an initial ATP concentration of at least 1 nM, 1.1. nM, 1.2 nM, 1.3 nM, 1.4 mM, 1.5 mM, 1.6 mM, 2 mM, 2.5 mM, 3 nM, 3.5 nM, 4 mM, 5 mM, 10 mM, 20 mM, 30 mM, or 50 mM. ATP concentration can be evaluated by a variety of means including, in certain embodiments, a BacTiter-Glo™ assay (Promega) on lysed ADAS.

High activity may be additionally or alternatively assessed as the rate or amount of increase in ATP concentration in an ADAS over time. In some embodiments, the ATP concentration of an ADAS is increased by at least 50%, at least 60%, at least 75%, at least 100%, at least 150%, at least 200%, or more than 200% following incubation under suitable conditions, e.g., incubation at 37° C. for 12 hours. In certain embodiments, a highly active ADAS has a rate of ATP generation greater than about: 0.000001, 0.00001, 0.0001, 0.001, 0.01, 0.05, 0.1, 0.5, 1.0, 2, 3, 5, 10, 15, 20, 30, 40, 50, 75, 100, 200, 300, 500, 1000, 10000 ATP/see/nm² for at least about: 1 hour, 2 hours, 3 hours, 4 hours, 5 hours, 6 hours, 12 hours, 1 day, 2 days, 4 days, 1 week, or two weeks.

In other aspects, high activity is assessed as a rate of decrease in ATP concentration over time. In some embodiments, ATP concentration may decrease less rapidly in ADAS that are highly active than in ADAS that are not highly active. In some embodiments, the drop in ATP concentration in an ADAS or an ADAS composition at 24 hours after preparation is less than about 50% (e.g., less than about: 45, 40, 35, 30, 25, 20, 15, 10, or 5%) compared to the initial ATP concentration (e.g., ATP per cell volume), e.g., as measured using a BacTiter-Glo™ assay (Promega).

High activity may be additionally or alternatively assessed as lifetime index of an ADAS. The lifetime index is calculated as the ratio of the rate of GFP production at 24 hours vs. 30 minutes. In some embodiments, a highly active ADAS has a lifetime index of greater than about: 0.13, 0.14, 0.15, 0.16, 0.18, 0.2, 0.25, 0.3,0.35, 0.45, 0.5, 0.60, 0.70, 0.80, 0.90, 1.0 or more. In more particular embodiments, lifetime index is measured in an ADAS containing a functional GFP plasmid with a species-appropriate promoter in which GFP concentration is measured relative to number of ADAS, average number of plasmids per ADAS, and solution volume with a plate reader at 30 minutes and 24 hours.

In some aspects, the ADAS produces a protein, e.g., a heterologous protein. In some aspects, high activity is assessed as a rate, amount, or duration of production of a protein or a rate of induction of expression of the protein (e.g., responsiveness of an ADAS to a signal). For example, the ADAS may comprise a plasmid, the plasmid comprising an inducible promoter and a nucleotide sequence encoding the heterologous protein, wherein contacting the ADAS with an inducer of the inducible promoter under appropriate conditions results in production of the heterologous protein. In some aspects, the production of the heterologous protein is increased by at least 1.6-fold in an ADAS, e.g., a highly active ADAS, that has been contacted with the inducer relative to an ADAS that has not been contacted with the inducer. For example, in some embodiments, the production of the heterologous protein is increased by at least 1.5-fold, 1.75-fold, 2-fold, 3-fold, 4-fold, 5-fold, 10-fold, or more than 10-fold in an ADAS, e.g., a highly active ADAS, that has been contacted with the inducer. In some embodiments, the rate of production of the heterologous protein by a highly active ADAS reaches a target level within a particular duration following the contacting of the ADAS with the inducer, e.g., within 5 minutes, 10 minutes, 15 minutes, 30 minutes, 45 minutes, 1 hour, 2 hours, 3 hours, or more than 3 hours. In some embodiments, a protein (e.g., a heterologous protein) is produced at a rate of at least 0.1 femtograms per hour per highly active ADAS, e.g, at least 0.2 0.4, 0.6, 0.8, 1, 2, 4, 6, 8, 10, 25, 50, 100, 250, 500, 1000, 2000, 3000, or 3500 fg/hour per ADAS. In some embodiments, high activity of an ADAS is assessed as a duration for which a protein is produced. A highly active ADAS may produce a protein (e.g. a heterologous protein) for a duration of at least 2 hours, at least 4 hours, at least 8 hours, at least 12 hours, at least 24 hours, at least 48 hours, or longer than 48 hours.

B. ADAS And Highly Active ADAS Derived from Parent Bacteria Deficient in a Cell Division Topological Specificity Factor

ADAS may be derived from bacterial parent cells, as described herein.

In some aspects, the invention provides an ADAS and/or a composition comprising a plurality of ADAS derived from a parent bacterium having a reduction in a level, activity, or expression of a cell division topological specificity factor.

In some aspects, the invention provides a composition comprising a plurality of ADAS, wherein the ADAS do not comprise a cell division topological specificity factor and wherein the composition is substantially free of viable bacterial cells.

In some aspects, the invention provides a composition comprising a plurality of ADAS, the composition being substantially free of viable bacterial cells, and being produced by a process comprising: (a) making, providing, or obtaining a plurality of parent bacteria having a reduction in the level or activity of a cell division topological specificity factor; (b) exposing the parent bacterium to conditions allowing the formation of a minicell, thereby producing the highly active ADAS; and (c) separating the ADAS from the parent bacterium, thereby producing a composition that is substantially free of viable bacterial cells.

In some embodiments of the above aspects, the cell division topological specificity factor is a polypeptide having an amino acid sequence with at least 20% identity to an E. coli minE polypeptide (SEQ ID NO: 1), e.g., at least 30%, 40%, 50%, 60%, 70%, 80%, 90%, 95%, 98%, or 99% identity to SEQ ID NO: 1. In some embodiments, the cell division topological specificity factor comprises the amino acid sequence of SEQ ID NO: 1. In some embodiments, the cell division topological specificity factor is a minE polypeptide. Exemplary species having minE polypeptides are provided in Table 4 and in Rothfield et al., Nature Reviews Microbiology, 3:959-968, 2005.

In some embodiments, the parent bacterium is E. coli and the minE polypeptide is E. coli minE. In other embodiments, the parent bacterium is Salmonella typhimurium and the minE polypeptide is S. typhimurium minE. In yet other embodiments, the parent bacterium is an Escherichia, Acinetobacter, Agrobacterium, Anabaena, Aquifex, Azoarcus, Azotobacter, Bordetella, Bradyrhizobium, Brucella, Buchnera, Burkholderia, Candidatus, Chromobacterium, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylococcus, Neisseria, Nitrosomonas, Nostoc, Photobacterium, Photorhabdus, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Symbiobacterium, or Thermoanaerobacter bacterium and the cell division topological specificity factor is the endogenous minE or DivIVA of the parent bacterium.

In some embodiments of the above aspects, the cell division topological specificity factor is a polypeptide having an amino acid sequence with at least 20% identity to a Bacillus subtilis DivIVA polypeptide (SEQ ID NO: 4), e.g., at least 30%, 40%, 50%, 60%, 70%, 80%, 90%, 95%, 98%, or 99% identity to SEQ ID NO: 4. In some embodiments, the cell division topological specificity factor comprises the amino acid sequence of SEQ ID NO: 4. In some embodiments, the cell division topological specificity factor is a DivIVA polypeptide. Exemplary species having DivIVA polypeptides are provided in Table 4 and in Rothfield et al., Nature Reviews Microbiology, 3:959-968, 2005. In some embodiments, the parent bacterium is Bacillus subtilis and the cell division topological specificity factor is B. subtilis DivIVA.

In some embodiments, the ADAS or parent bacterium having the reduction in a level or activity of the cell division topological specificity factor also has a reduction in a level of one or more Z-ring inhibition proteins.

In some embodiments, the Z ring inhibition protein is a polypeptide having an amino acid sequence with at least 20% identity to an E. coli minC polypeptide (SEQ ID NO: 2), e.g., at least 30%, 40%, 50%, 60%, 70%, 80%, 90%, 95%, 98%, or 99% identity to SEQ ID NO: 2. In some embodiments, the Z ring inhibition protein comprises the amino acid sequence of SEQ ID NO: 2. In some embodiments, the Z ring inhibition protein is a minC polypeptide.

In some embodiments, the Z ring inhibition protein is a polypeptide having an amino acid sequence with at least 20% identity to an E. coli minD polypeptide (SEQ ID NO: 3), e.g., at least 30%, 40%, 50%, 60%, 70%, 80%, 90%, 95%, 98%, or 99% identity to SEQ ID NO: 3. In some embodiments, the Z ring inhibition protein comprises the amino acid sequence of SEQ ID NO: 3. In some embodiments, the Z ring inhibition protein is a minD polypeptide.

In some embodiments, the ADAS or parent bacterium has a reduction in the level, activity, or expression of at least two Z-ring inhibition proteins. In some embodiments, the ADAS or parent bacterium has a reduction in expression of a minC polypeptide and a minD polypeptide. In some embodiments, the ADAS or parent bacterium has a reduction in expression of a minC polypeptide, a minD polypeptide, and a minE polypeptide, e.g., a deletion of the minCDE operon (AminCDE).

A reduction in the level, activity, or expression of a cell division topological specificity factor or a Z-ring inhibition protein, e.g., a reduction in an ADAS or a reduction in a parent bacterial cell, may be achieved using any suitable method. For example, in some embodiments, the reduction in the level or activity is caused by a loss-of-function mutation, e.g., a gene deletion. In some embodiments, the loss-of-function mutation is an inducible loss-of-function mutation and loss of function is induced by exposing the parent cell to an inducing condition, e.g., the inducible loss-of-function mutation is a temperature-sensitive mutation and wherein the inducing condition is a temperature condition.

In some embodiments, the parent cell has a deletion of the minCDE operon (AminCDE) or homologous operon.

### C. ADAS Comprising a Cargo

In some embodiments, an ADAS provided by the invention includes a cargo contained in the interior of the ADAS. A cargo may be any moiety disposed in the interior of an ADAS (e.g., encapsulated by the ADAS) or conjugated to the surface of the ADAS. In some embodiments, the cargo comprises a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP) or a combination of the foregoing.

In some embodiments, the nucleic acid is a DNA, an RNA, or a plasmid. In some embodiments, the nucleic acid (e.g., DNA, RNA, or plasmid) encodes a protein. In some embodiments the protein is transcribed and/or translated in the ADAS.

In some embodiments, the cargo is an enzyme. In some embodiments, the enzyme alters a substrate to produce a target product. In some embodiments, the substrate is present in the ADAS and the target product is produced in the ADAS. In other embodiments, the substrate is present in a target cell or environment to which the ADAS is delivered.

In certain embodiments, the cargo is modified for improved stability compared to an unmodified version of the cargo. “Stability” of a cargo is a unitless ratio of half-life of unmodified cargo and modified cargo half-life, as measured in the same environmental conditions. In some embodiments the environment is experimentally controlled, e.g., a simulated body fluid, RNAse free water, cell cytoplasm, extracellular space, or “ADAS plasm” (i.e., the content of the interior volume of an ADAS, e.g., after lysis). In some applications it is an agricultural environment, e.g., variable field soil, river water, or ocean water. In other embodiments, the environment is an actual or simulated: animal gut, animal skin, animal reproductive tract, animal respiratory tract, animal blood stream, or animal extracellular space. In certain embodiments, the ADAS does not substantially degrade the cargo.

In certain embodiments, the cargo comprises a protein. In certain embodiments, the protein has stability greater than about: 1.01, 1.1, 10, 100, 1000, 10000, 100000, 100000, 10000000 in cell cytoplasm or other environments. The protein can be any protein, including growth factors; enzymes; hormones; immune-modulatory proteins; antibiotic proteins, such as antibacterial, antifungal, insecticide, proteins, etc.; targeting agents, such as antibodies or nanobodies, etc. In some embodiments, the protein is a hormone, e.g., paracrine, endocrine, autocrine.

In some embodiments, the cargo comprises a plant hormone, such as abscisic acid, auxin, cytokinin, ethylene, gibberellin, or a combination thereof.

In certain embodiments, the cargo is an immune modulator, such as an immune stimulator, check point inhibitors (e.g., of PD-1, PD-L1, CTLA-4), chemotherapeutic agent, suppressors, super antigens, small molecule (cyclosporine A, cyclic dinucleotides (CDNs), or STING agonist (e.g., MK-1454)).

For ADAS comprising cargo, in some embodiments, the cargo is an RNA, such as circular RNA, mRNA, siRNA, shRNA, ASO, IRNA, dsRNA, or a combination thereof. In certain embodiments the RNA has stability greater than about: 1.01, 1.1,10, 100, 1000, 10000, 100000, 100000, 10000000, e.g., in ADAS plasm. The RNA cargo can be stabilized, in certain embodiments, e.g., with an appended step-loop structure, such as a tRNA scaffold. For example, non-human tRNALys3 and E. coli tRNAMet (Nat. Methods, Ponchon 2007). Both have been well characterized and expressed recombinantly. However, a variety of other types could be used as well, such as aptamers, lncRNA, ribozymes, etc., RNA can also be stabilized where're the ADAS is obtained from a parental strain null (or hypomorphic) for one or more ribonucleases.

In some particular embodiments, the RNA is a protein-coding mRNA. In more particular embodiments, the protein-coding mRNA encodes an enzyme (e.g., and enzyme that imparts hepatic enzymatic activity, such as human PBGD (hPBGD) mRNA) or an antigen, e.g., that elicits an immune response (such as eliciting a potent and durable neutralizing antibody titer), such as mRNA encoding CMV glycoproteins gB and/or pentameric complex (PC)). In certain particular embodiments, the RNA is a small non-coding RNA, such as shRNA, ASO, RNA, dsRNA, or a combination thereof.

In certain embodiments, the ADAS provided by the invention includes cargo comprising a gene editing system. A “gene editing system” includes (or encodes) proteins that can, with suitable associated nucleic acids, modify a DNA sequence of interest, such as a genomic DNA sequence, whether by insertion or deletion of a sequence of interest, as well as an altered methylation state. Exemplary gene editing systems include those based on a Cas system, such as Cas9, Cpf1 or other RNA-targeted systems with their companion RNA, as well as Zinc finger nucleases and TAL-effectors conjugated to nucleases.

Other embodiments of ADAS provided by the invention include DNA as the cargo, including as a plasmid, optionally wherein the DNA comprises a protein-coding sequence. Exemplary DNA cargo includes, in certain embodiments, a plasmid encoding an RNA sequence of interest (see examples above), e.g., which may be flanked on each side by an tRNA insert. Various DNA cargo are encompassed by the invention, including: ADAS producing (e.g., driving FTZ overexpression, genome degrading exonucleases); longevity plasmids (ATP synthase expressing, rhodopsin-expressing); those expressing stabilized non-coding RNA, tRNA, lncRNA; expressing secretion system tag proteins, NleE2 effector domain and localization tag; secretion systems T3/4SS, T5SS, T6SS; logic circuits, conditionally expressed secretion systems; and combinations thereof. In some embodiments, a logic circuit includes inducible expression or suppression cassettes, such as IPTG-inducible Plac promoter and the hrpR portion of the AND gate, and, for example, the heat-induced promoter pL (from phage lambda, which is usually suppressed by a thermolabile protein) and the hrpS portion of the AND gate. To engineer an OR gate, a sytem described by Rosado et al., PLOS Genetics, 2018 can be used. Briefly, a cis-repressed mRNA coding for RFP under a constitutive promoter can be used. The repression can then be removed in the presence of RAJ11 sRNA. Plasmids containing the IPTG-inducible promoter PLac and heat-induced promoter pL, both of which induce the expression of RAJ11 sRNA, can then be used. The output would then be RFP expression, which is seen in response to either input. These systems can be adapted to a variety of sensor-type functions.

ADAS provided by the invention, in some embodiments, include a transporter in the membrane. In certain embodiments, the transporter is specific for glucose, sodium, potassium, a metal ion, an anionic solute, a cationic solute, or water.

In some embodiments, the membrane of an ADAS provided by the invention comprises an enzyme. In particular embodiments, the enzyme is a protease, oxidoreductase, or a combination thereof. In some embodiments, the enzyme is chemically conjugated to the ADAS membrane, optionally via a linker to the exterior membrane.

### D. ADAS Comprising a Secretion System

In certain embodiments, an ADAS provided by the invention comprises a bacterial secretion system (e.g., an endogenous bacterial secretion system or a heterologous secretion system). A “bacterial secretion system” is a protein, or protein complex, that can export a cargo from the cytoplasm of a bacterial cell (or, for example, an ADAS derived therefrom) into: the extracellular space, the periplasmic space of a gram-negative bacterium, or the intracellular space of another cell. In some embodiments, the bacterial secretion system works by an active (e.g., ATP-dependent or PMF-dependent) process, and in certain embodiments the bacterial secretion system comprises a tube or a spike spanning the host cell (or ADAS) to a target cell. In other embodiments the bacterial secretion system is a transmembrane channel. Exemplary bacterial secretion systems include T3SS and T4SS (and T3/T4SS, as defined, below), which are tube-containing structures where the cargo traverses through the inside of a protein tube and T6SS, which carries the cargo at the end of a spike. Other exemplary bacterial secretion systems include T1SS, T2SS, T5SS, T7SS, Sec, and Tat, which are transmembrane.

In some embodiments, the heterologous secretion system is a T3SS.

In some embodiments, the ADAS comprises a cargo, wherein the cargo comprises a moiety that directs export by the bacterial secretion system, e.g., in some embodiments the moiety is Pho/D, Tat, or a synthetic peptide signal.

In certain embodiments, the ADAS provided by the invention are two-membrane ADAS. In more particular embodiments the two-membrane ADAS further comprises a bacterial secretion system. In still more particular embodiments, the bacterial secretion system is selected from T3SS, T4SS, T3/4SS, or T6SS, optionally wherein the T3SS, T4SS, T3/4SS, or T6SS have an attenuated or non-functional effector that does not affect fitness of a target cell.

ADAS provided by the invention, in some embodiments, include a bacterial secretion system.

In some embodiments, the bacterial secretion system is capable of exporting a cargo across the ADAS outer membrane into a target cell, such as an animal, fungal, bacterial, or plant cell, such as T3SS, T4SS, T3/T4SS, or T6SS.

In more particular embodiments, the bacterial secretion system is a T3/4SS. A “T3/4SS” is a secretion system based on T3SS or T4SS, including hybrid systems as well as unmodified versions, which forms a protein tube between a bacterium (or ADAS) and a target cell, connecting the two and delivering one or more effectors. The target cell can be an animal, plant, fungi, or bacteria. In some embodiments a T3/4SS includes an effector, which may be a modified effector. Examples of T3SS systems include the Salmonella SPI-1 system, the EHEC coli ETT1 system, the Xanthamonas Citri/Campestri T3SS system, and the Pseudomonas syringae T3SS system. Examples of T4SS systems include the Agrobacterium Ti plasmid system, Helicobacter pylori T4SS. In certain embodiments, the T3/4SS has modified effector function, e.g., an effector selected from SopD2, SopE, Bop, Map, Tir, EspB, EspF, NleC, NieH2, or NleE2. In more particular embodiments, the modified effector function is for intracellular targeting such as translocation into the nucleus, golgi, mitochondria, actin, microvilli, ZO-1, microtubules, or cytoplasm. In still more particular embodiments, the modified effector function is nuclear targeting based on NleE2 derived from E. Coli. In other particular embodiments, the modified effector function is for filopodia formation, tight junction disruption, microvilli effacement, or SGLT-1 inactivation.

In other embodiments, an ADAS provided by the invention comprising a bacterial secretion system comprises a T6SS. In some embodiments, the T6SS, in its natural host, targets a bacterium and contains an effector that kills the bacteria. In certain particular embodiments, the T6SS is derived from P. putida K1-T6SS and, optionally, wherein the effector comprises the amino acid sequence of Tke2 (Accession AUZ59427.1), or a functional fragment thereof. In other embodiments, the T6SS, in its natural host, targets a fungi and contains an effector that kills fungi, e.g., the T6SS is derived from Serratia Marcescens and the effectors comprise the amino acid sequences of: Tfe1 (Genbank: SMDB11_RS05530) or Tfe2 (Genbank: SMDB11_RS05390).

In other embodiments of an ADAS provided by the invention that contains a bacterial secretion system, the bacterial secretion system is capable of exporting a cargo extracellularly. In certain more particular embodiments, the bacterial secretion system is T1SS, T2SS, T5SS, T7SS, Sec, or Tat.

E. ADAS Lacking Proteases, RNases, and/or LPS

In another aspect, the invention provides a composition comprising a plurality of ADAS (e.g., highly active ADAS), wherein the ADAS have a reduced protease level or activity relative to an ADAS produced from a wild-type parent bacterium. In some aspects, the ADAS is produced from a parent bacterium that has been modified to reduce or eliminate expression of at least one protease.

In another aspect, the invention provides a composition comprising a plurality of ADAS (e.g., highly active ADAS), wherein the ADAS have a reduced RNAse level or activity relative to an ADAS produced from a wild-type parent bacterium. In some aspects, the ADAS is produced from a parent bacterium that has been modified to reduce or eliminate expression of at least one RNAse. In some embodiments, the RNase is an endoribonuclease or an exoribonuclease.

In another aspect, the invention provides a composition comprising a plurality of ADAS, wherein the ADAS has been modified to have reduced lipopolysaccharide (LPS). In some embodiments, the modification is a mutation in Lipid A biosynthesis myristoyltransferase (msbB).

In certain embodiments, an ADAS provided by the invention lacks one or more metabolically non-essential proteins. A “metabolically non-essential protein” non-exhaustively includes: fimbriae, flagella, undesired secretion systems, transposases, effectors, phage elements, or their regulatory elements, such as flhC or OmpA. In some embodiments, an ADAS provided by the invention lacks one or more of an RNAse, a protease, or a combination thereof, and, in particular embodiments, lacks one or more endoribonucleases (such as RNAse A, RNAse h, RNAse III, RNAse L, RNAse PhyM) or exoribonucleases (such as RNAse R, RNAse PH, RNAse D); or serine, cysteine, threonine, aspartic, glutamic and metallo-proteases; or a combination of any of the foregoing.

### E. ADAS Comprising a Targeting Moiety

In another embodiment, the invention provides a composition comprising a plurality of ADAS (e.g., highly active ADAS), wherein the ADAS comprises a targeting moiety. In some embodiments, the targeting moiety is a nanobody, a carbohydrate binding protein, or a tumor-targeting peptide.

In certain embodiments, the nanobody, is nanobody directed to a tumor antigen, such as HER2, PSMA, or VEGF-R. In other embodiments, the carbohydrate binding protein is a lectin, e.g. Mannose Binding Lectin (MBL). In still other embodiments, the tumor-targeting peptide is an RGD motifs or CendR peptide.

F. ADAS Derived from Commensal or Pathogenic Parent Strains

In another embodiment, the invention provides a composition comprising a plurality of ADAS (e.g., highly active ADAS), wherein the ADAS is derived from a parent bacterium that is a mammalian pathogen or a mammalian commensal bacterium. In some instances, the mammalian commensal bacterium is a Staphylococcus, Bifidobacterium, Micrococcus, Lactobacillus, or Actinomyces species or the mammalian pathogenic bacterium is enterohemorrhagic Escherichia coli (EHEC), Salmonella typhimurium, Shigella flexneri, Yersinia enterolitica, or Helicobacter pylori.

In another embodiment, the invention provides a composition comprising a plurality of ADAS (e.g., highly active ADAS), wherein the ADAS is derived from a parent bacterium that is a plant pathogen or a plant commensal bacterium. In some instances, the plant commensal bacterium is Bacillus subtilis or Psuedomonas putida or the plant pathogenic bacterium is a Xanthomonas species or Pseudomonas syringae.

G. ADAS Derived from Auxotrophic Parent Strains

In another embodiment, the invention provides a composition comprising a plurality of ADAS (e.g., highly active ADAS), wherein the ADAS is derived from an auxotrophic parent bacterium, i.e., a a parent bacterium that is unable to synthesize an organic compound required for growth. Such bacteria are able to grow only when the organic compound is provided.

### H. ADAS Comprising Additional Moieties

An ADAS, in certain embodiments, includes a functional ATP synthase and, in some embodiments, a membrane embedded proton pump. ADAS can be derived from different sources including: a parental bacterial strain (“parental strain”) engineered or induced to produce genome-free enclosed membrane systems, a genome-excised bacterium, a bacterial cell preparation extract (e.g., by mechanical or other means), or a total synthesis, optionally including fractions of a bacterial cell preparation. In some embodiments, a highly active ADAS has an ATP synthase concentration of at least: 1 per 10000 nm², 1 per 5000 nm², 1 per 3500 nm², 1 per 1000 nm².

ADAS provided by the invention can include a variety of additional components, including, for example, photovoltaic pumps, retinals and retinal-producing cassettes, metabolic enzymes, targeting agents, cargo, bacterial secretion systems, and transporters, including combinations of the foregoing, including certain particular embodiments described, below. In certain embodiments, the ADAS lack other elements, such as metabolically non-essential genes and/or certain nucleases or proteases.

In certain embodiments, the an ADAS provided by the invention comprises an ATP synthase, optionally lacking a regulatory domain, such as lacking an epsilon domain. Deletion can be accomplished by a variety of means. In certain embodiments, the deletion in by inducible deletion of the native epsilon domain. In certain embodiments, deletion can be accomplished by flanking with LoxP sites and inducible Cre expression or CRISPR knockout, or be inducible (place on plasmid under a tTa tet transactivator in an ATP synthase knockout strain)

An ADAS, in some embodiments, can include a photovoltaic proton pump. In certain embodiments, the photovoltaic proton pump is a proteorhodopsin. In more particular embodiments, the proteorhodopsin comprises the amino acid sequence of proteorhodopsin from the uncultured marine bacterial clade SAR86, GenBank Accession: AAS73014.1. In other embodiments, the photovoltaic proton pump is a gloeobacter rhodopsin. In certain embodiments, the photovoltaic proton pump is a bacteriorhodopsin, deltarhodopsin, or halorhodopsin from Halobium salinarum Natronomonas pharaonis, Exiguobacterium sibiricum, Haloterrigena turkmenica, or Haloarcula marismortui.

In some embodiments, an ADAS provided by the invention further comprising retinal. In certain embodiments, an ADAS provided by the invention further comprises a retinal synthesizing protein (or protein system), or a nucleic acid encoding the same.

In certain embodiments, an ADAS provided by the invention further comprises one or more glycolysis pathway proteins. In some embodiments, the glycolysis pathway protein is a phosphofructokinase (Pfk-A), e.g., comprising the amino acid sequence of UniProt accession POA796 or a functional fragment thereof. In other embodiments the glycolysis pathway protein is triosephosphate isomerase (tpi), e.g., comprising the amino acid sequence of UniProt accession POA858, or a functional fragment thereof.

### I. ADAS Compositions and Formulations

The present invention provides compositions or preparations that contain an ADAS provided by the invention, including, inter alia, a highly active ADAS preparation provided by the invention or an ADAS preparation wherein a plurality of individual ADAS lack a cell division topological specificity factor, e.g., lack a minE gene product, and optionally wherein the ADAS preparation is substantially free of viable cells. Collectively, these are “compositions provided by the invention” or “a composition provided by the invention”, or the like and can contain any ADAS provided by the invention and any combination of ADAS provided by the invention.

For example, in some embodiments, a composition provided by the invention contains at least about: 80, 81, 82, 83, 84, 85, 90, 95, 96, 97, 98, 99, 99.1, 99.2, 99.3, 99.4, 99.5, 99.6, 99.7, 99.8, 99.9%, or more ADAS that contain a bacterial secretion system. In particular embodiments, the bacterial secretion system is one of T3SS, T4SS, T3/4SS, or T6SS.

In some embodiments, a composition provided by the invention contains ADAS that contain a T3SS, where the ADAS comprise a mean T3SS membrane density greater than 1 in about: 40000, 35000,30000, 25000, 19600, 15000, 10000, or 5000 nm². In certain particular embodiments, the ADAS is derived from a S. typhimurium or E. coli parental strain.

Certain embodiments of the compositions provided by the invention contain ADAS that contain a T3SS, where the ADAS comprise a mean T3SS membrane density greater than 1 in about: 300000, 250000, 200000, 150000, 100000, 50000, 20000, 10000, 5000 nm². In certain particular embodiments, the ADAS is derived from an Agrobacterium tumefaciens parental strain.

In another aspect, the invention provide a composition of ADAS, wherein at least about: 80, 81, 82, 83, 84, 85, 90, 95, 96, 97, 98, 99, 99.1, 99.2, 99.3, 99.4, 99.5, 99.6, 99.7, 99.8, 99.9%, or more of the ADAS contain a bacterial secretion system, including T3, T4, T3/4SS, T6SS, and optionally including one or more of: exogenous carbohydrates, phosphate producing synthases, light responsive proteins, import proteins, enzymes, functional cargo, organism-specific effectors, fusion proteins.

As will be readily apparent the compositions and preparations provided by the invention can contain any ADAS provided by the invention, such as highly active ADAS or ADAS that lack a minE gene product.

The compositions provided by the invention can be prepared in any suitable formulation. For example, the formulation can be suitable for IP, IV, IM, oral, topical (cream, gel, ointment, transdermal patch), aerosolized, or nebulized administration. In some embodiments, a formulation is a liquid formulation. In other embodiments, the formulation is a lyophilized formulation.

In some embodiments, an ADAS composition described herein comprises less than 100 colony-forming units (CFU/mL) of viable bacterial cells, e.g., less than 50 CFU/mL, less than 20 CFL/mL, less than 10 CFU/mL, less than 1 CFU/mL, or less than 0.1 CFU/mL of viable bacterial cells.

In some embodiments, the invention provides an ADAS composition wherein the ADAS are lyophilized and reconstituted, and wherein the reconstituted ADAS have an ATP concentration that is at least 90% of the ATP concentration of an ADAS that has not been lyophilized, e,g, at least 95%, 98%, or at least equal to the ATP concentration of an ADAS that has not been lyophilized.

In some embodiments, the invention provides an ADAS composition wherein the ADAS are stored, e.g., stored at 4° C., wherein after storage, the ADAS have an ATP concentration that is at least 90% of the ATP concentration of an ADAS that has not been stored, e.g., at least 95%, 98%, or at least equal to the ATP concentration of an ADAS that has not been stored. In some embodiments, the storage is for at least one day, at least one week, at least two weeks, at least three weeks, at least one month, at least two months, at least six months, or at least a year.

In some embodiments, ADAS may be preserved or otherwise in a “quiescent” state and then rapidly become activated.

In some embodiments, the ADAS composition is formulated for delivery to an animal, e.g., formulated for intraperitoneal, intravenous, intramuscular, oral, topical, aerosolized, or nebulized administration.

In some embodiments, the ADAS composition is formulated for delivery to a plant.

In some embodiments, the ADAS composition is formulated for delivery to an insect.

In some embodiments, the composition is formulated as a liquid, a solid, an aerosol, a paste, a gel, or a gas composition.

### J. ADAS Comprising an Enzyme

In one aspect, the invention features a composition comprising a plurality of ADAS, wherein the ADAS comprise an enzyme and wherein the enzyme alters a substrate to produce a target product. In some embodiments, the substrate is present in the ADAS and the target product is produced in the ADAS. In other embodiments, the substrate is present in a target cell or environment to which the ADAS is delivered. In some embodiments, the enzyme is diadenylate cyclase A, the substrate is ATP, and the target product is cyclic-di-AMP.

### II. Methods of Manufacturing Adas

### A. Making ADAS and Highly Active ADAS

In some aspects, the invention features a method for manufacturing a composition comprising a plurality of ADAS, the composition being substantially free of viable bacterial cells, the method comprising (a) making, providing, or obtaining a plurality of parent bacteria having a reduction in the level or activity of a cell division topological specificity factor; (b) exposing the parent bacteria to conditions allowing the formation of a minicell, thereby producing the highly active ADAS; and (c) separating the highly active ADAS from the parent bacteria, thereby producing a composition that is substantially free of viable bacterial cells.

Parent bacteria include any suitable bacterial species from which an ADAS may be generated (e.g., species that may be modified using methods described herein to produce ADAS). Table 4 provides a non-limiting list of suitable genera from which ADAS can be derived.

TABLE 4
Bacterial species for ADAS production

Gram negative bacteria
Escherichia
Acinetobacter
Agrobacterium
Anabaena
Aquifex
Azoarcus
Azotobacter
Bordetella
Bradyrhizobium
Brucella
Buchnera
Burkholderia
Candidatus
Chromobacterium
Crocosphaera
Dechloromonas
Desulfitobacterium
Desulfotalea
Erwinia
Francisella
Fusobacterium
Gloeobacter
Gluconobacter
Helicobacter
Legionella
Magnetospirillum
Mesorhizobium
Methylococcus
Neisseria
Nitrosomonas
Nostoc
Photobacterium
Photorhabdus
Polaromonas
Prochlorococcus
Pseudomonas
Psychrobacter
Ralstonia
Rubrivivax
Salmonella
Shewanella
Shigella
Sinorhizobium
Synechococcus
Synechocystis
Thermosynechococcus
Thermotoga
Thermus
Thiobacillus
Trichodesmium
Vibrio
Wigglesworthia
Wolinella
Xanthomonas
Xylella
Yersinia
Gram positive bacteria
Bacillus
Clostridium
Deinococcus
Exiguobacterium
Geobacillus
Lactobacillus
Listeria
Moorella
Oceanobacillus
Symbiobacterium
Thermoanaerobacter

In some aspects, the invention features methods for manufacturing any of the ADAS compositions, e.g., highly active ADAS compositions, described in Section I herein. For example, provided herein are methods for making highly active ADAS; methods for making ADAS lacking a cell division topological specificity factor and, optionally, lacking a Z-ring inhibition protein (e.g., methods of making ADAS from AminCDE parent bacteria), and methods for making any of the ADAS mentioned herein, wherein the ADAS comprises a cargo.

The highly active ADAS of any one of the preceding claims may be made from a bacterial cell, wherein the parental strain is selected from a plant bacterium, such as a plant commensal (e.g., B. Subtilis or Pseudomonas putida) or a plant pathogen bacterium (e.g., Xanthomonas sp. Or Psuedomonas syringae) or a human bacterium, such as a commensal human bacterium (e.g., E. coli, Staphylococcus sp., Bifidobacterium sp., Micrococcus sp., Lactobacillus sp., or Actinomyces sp.) or a pathogenic human bacterium (e.g., Escherichia coli EHEC, Salmonella Typhimurium, Shigella flexneri, Yersinia enterolitica, Helicobacter pylori), or an extremophile, including functionalized derivatives of any of the foregoing, for example including a functional cassette, such as a functional cassette that induces the bacterium to do one or more of: secrete antimicrobials, digest plastic, secrete insecticides, survives extreme environments, make nanoparticles, integrate within other organisms, respond to the environment, and create reporter signals.

Parent bacteria may include functionalized derivatives of any of the foregoing, for example including a functional cassette, such as a functional cassette that induces the bacterium to do one or more of: secrete antimicrobials, digest plastic, secrete insecticides, survives extreme environments, make nanoparticles, integrate within other organisms, respond to the environment, and create reporter signals.

In some embodiments, an ADAS is derived from a parental strain engineered or induced to overexpress ATP synthase. In some more particular embodiments, the ATP synthase is heterologous to the parental strain. In certain particular embodiments, the parental strain is modified to express a functional FoF1 ATP synthase.

In certain embodiments, an ADAS provided by the invention is obtained from a parental strain cultured under a condition selected from: applied voltage (e.g., 37 mV), non-atmospheric oxygen concentration (e.g., 1-5% O₂, 5-10% O₂, 10-15% O₂, 25-30% O₂), low pH (about: 4.5, 5.0, 5.5, 6.0, 6.5), or a combination thereof.

The highly active ADAS of any one of the preceding claims, which is made from an extremophile, including functionalized derivatives of any of the foregoing, for example including a functional cassettes, such as a functional cassette that induces the bacterium to do one or more of: secrete antimicrobials, digest plastic, secrete insecticides, survives extreme environments, make nanoparticles, integrate within other organisms, respond to the environment, and create reporter signals.

Owing to the diversity of bacterium, ADAS can be made with modified membranes, e.g., to improve the biodistribution of the ADAS upon administration to a target cell. In certain embodiments, the membrane is modified to be less immunogenic or immunostimulatory in plants or animals. For example, in certain embodiments, the ADAS is obtained from a parental strain, wherein the immunostimulatory capabilities of the parental strain are reduced or eliminated through post production treatment with detergents, enzymes, or functionalized with PEG. In certain embodiments the ADAS is made from a parental strain and the membrane is modified through knockout of LPS synthesis pathways in the parental strain, e.g., by knocking out msbB. In other particular embodiments, the ADAS is made from a parental strain that produces cell wall-deficient particles through exposure to hyperosmotic conditions. In some embodiments, the methods include transforming a parental strain with an inducible DNAse system, such as the exol (NCBI GeneID: 946529) & sbcD (NCBI GeneID: 945049) nucleases, or the I-Ceul (e.g., Swissprot: P32761.1) nuclease. In more particular embodiments, the methods include using a single, double, triple, or quadruple auxotrophic strain and having the complementary genes on the plasmid encoding the inducible nucleases.

In some embodiments, the methods of the methods provided by the invention, the parental strain is cultured under a condition selected from: applied voltage (e.g., 37 mV), non-atmospheric oxygen concentration (e.g., 1-5% O₂, 5-10% O₂, 10-15% O₂, 25-30% O₂), low pH (4.5-6.5), or a combination thereof.

In certain embodiments, the methods of the methods provided by the invention, parental strain lacks flagella and undesired secretion systems, optionally where the flagella and undesired secretion systems are removed using lambda red recombineering.

In some embodiments, the methods of provided by the invention, flagella control components are excised from the parental strain genome via, for example, insertion of a plasmid containing a CRISPR domain that is targeted towards flagella control genes, such as flhD and flhC.

In certain embodiments, the methods provided by the invention are for making a highly active ADAS, where an ADAS comprising a plasmid containing a rhodopsin-encoding gene is cultured in the presence of light. In more particular embodiments, the rhodopsin is proteorhodopsin from SAR86 uncultured bacteria, having the amino acid sequence of GenBank Accession: AAS73014.1, or a functional fragment thereof. In still more particular embodiments, the culture is supplemented with retinal. In other more particular embodiments, the rhodopsin is proteorhodopsin and the plasmid additionally contains genes synthesizing retinal (such a plasmid is the pACYC-RDS plasmid from Kim et al., Microb Cell Fact, 2012).

In certain particular embodiments, the parental strain contains a nucleic acid sequence encoding a nanobody that is then expressed on the membrane of the ADAS.

In some embodiments of the methods provided by the invention, the parental strain contains a nucleic acid sequence encoding one or more bacterial secretion system operons. Exemplary plasmids include the Salmonella SPI-1 T3SS, the Shigella flexneri T3SS, the Agro Ti plasmid, and the P. putida K1-T6SS system.

In certain embodiments, the parental strain comprises a cargo. In some embodiments, the parent strain contains a nucleic acid sequence encoding a set of genes that synthesize a small molecule cargo.

### IV. Purification of Adas and Adas Compositions

In some embodiments of the methods and compositions provided herein, ADAS are purified from compositions (e.g., cultures) comprising viable bacteria, e.g., parental bacteria. For example, the invention features a method for manufacturing a composition comprising a plurality of ADAS, the composition being substantially free of viable bacterial cells, the method comprising (a) making, providing, or obtaining a plurality of parent bacteria having a reduction in the level or activity of a cell division topological specificity factor; (b) exposing the parent bacteria to conditions allowing the formation of a minicell, thereby producing the highly active ADAS; and (c) separating the highly active ADAS from the parent bacteria, thereby producing a composition that is substantially free of viable bacterial cells.

Purification separates ADAS from viable parent bacterial cells, which are larger and contain a genome. Separating the highly active ADAS from the parent bacteria can be performed using a number of methods, as described herein. Exemplary methods for purification described herein include centrifugation, selective outgrowth, and buffer exchange/concentration processes.

In some aspects, provide herein are ADAS compositions, and methods of comparing such compositions, wherein the compositions are substantially free of parent bacterial cells and/or viable bacterial cells, e.g., have no more than 500, e.g., 400, 300, 200, 150, or 100 or fewer than 50, fewer than 25, fewer than 10, fewer than 5, fewer than 1, fewer than 0.1 colony-forming units (CFU) per mL. In some embodiments, an ADAS composition that is substantially free of parent bacterial cells may include no bacterial cells.

Auxotrophic parental strains can be used to make ADAS provided by the invention. As described in more detail below, such manufacturing methods are useful for purification of the ADAS. For example, following ADAS generation, parent bacterial cells may be removed by growth in media lacking the nutrient (for example, amino acid) necessary for viability of the parent bacterium. In some embodiments, an ADAS provided by the invention is derived from a parental strain auxotrophic for at least 1, 2, 3, 4, or more of: arginine (e.g., knockout in argA, such as strains JW2786-1 and NK5992), cysteine knockout in cysE (such as strains JW3582-2 and JM15), glutamine e.g., knockout in glnA (such as strains JW3841-1 and M5004), glycine e.g., knockout in glyA (such as strains JW2535-1 and AT2457), Histidine e.g., knockout in hisB (such as strains JW2004-1 and SB3930), isoleucine e.g., knockout in ilvA (such as strains JW3745-2 and AB1255), leucine e.g., knockout in leuB (such as strains JW5807-2 and CV514), lysine e.g., knockout in lysA (such as strains JW2806-1 and KL334), methionine e.g., knockout in metA (such as strains JW3973-1 and DL41), phenylalanine e.g., knockout in pheA (such as strains JW2580-1 and KA197), proline e.g., knockout in proA (such as strains JW0233-2 and NK5525), Serine e.g., knockout in serA (such as strains JW2880-1 and JC158), threonine e.g., knockout in thrC (such as strains JW0003-2 and Gif 41), tryptophan e.g., knockout in trpC (such as strains JW1254-2 and CAG18455), Tyrosine e.g., knockout in tyrA (such as strains JW2581-1 and N3087), Valine/Isoleucine/Leucine e.g., knockout in ilvd (such as strains JW5605-1 and CAG18431).

In certain embodiments, the methods include using a single, double, triple, or quadruple auxotrophic parental strain, optionally wherein said parental strain further includes a plasmid expressing a ftsZ.

### V. Methods of Using Adas

### A. Methods of Delivering an ADAS

In one aspect, the invention features a method for delivering a highly active ADAS to a target cell, the method comprising (a) providing a composition comprising a plurality of highly active ADAS, wherein the ADAS have an initial ATP concentration of at least 1.25 mM and wherein the composition is substantially free of viable bacterial cells; and (b) contacting the target cell with the composition of step (a).

In another aspect, the invention features a method for delivering an ADAS (e.g., a highly active ADAS) to a target cell, the method comprising: (a) providing a composition comprising a plurality of ADAS, wherein the ADAS are derived from a parent bacterium having a reduction in the level or activity of a cell division topological specificity factor and wherein the composition is substantially free of viable bacterial cells; and (b) contacting the target cell with the composition of step (a).

The target cell may be, e.g., an animal cell, a plant cell, or an insect cell.

### B. Methods of Delivering a Cargo

In another aspect, the invention features a method for delivering a cargo (e.g., a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)) to a target cell, the method comprising: (a) providing a composition comprising a plurality of highly active achromosomal dynamic active systems (ADAS), wherein the ADAS have an initial ATP concentration of at least 1.25 mM, the ADAS comprise a cargo, and the composition is substantially free of viable bacterial cells; and (b) contacting the target cell with the composition of step (a).

In another aspect, the invention features a method for delivering a cargo (e.g., a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)) to a target cell, the method comprising: (a) providing a composition comprising a plurality of ADAS, wherein the ADAS are derived from a parent bacterium having a reduction in the level or activity of a cell division topological specificity factor, the ADAS comprise a cargo, and the composition is substantially free of viable bacterial cells; and (b) contacting the target cell with the composition of step (a).

The target cell to which the cargo is delivered may be, e.g., an animal cell, a plant cell, or an insect cell.

### C. Methods of Modulating a State of a Cell

In one aspect, the invention features a method of modulating a state of an animal cell, the method comprising: (a) providing a composition comprising a plurality of highly active achromosomal dynamic active systems (ADAS), wherein the ADAS have an initial ATP concentration of at least 1.25 mM and wherein the composition is substantially free of viable bacterial cells; and (b) contacting the animal cell with the composition of step (a), whereby a state of the animal cell is modulated.

In another aspect, the invention features a method of modulating a state of a plant cell, the method comprising: (a) providing a composition comprising a plurality of highly active achromosomal dynamic active systems (ADAS), wherein the ADAS have an initial ATP concentration of at least 1.25 mM and wherein the composition is substantially free of viable bacterial cells; and (b) contacting the plant cell with the composition of step (a), whereby a state of the plant cell is modulated.

In another aspect, the invention features a method of modulating a state of an insect cell, the method comprising: (a) providing a composition comprising a plurality of highly active achromosomal dynamic active systems (ADAS), wherein the ADAS have an initial ATP concentration of at least 1.25 mM and wherein the composition is substantially free of viable bacterial cells; and (b) contacting the insect cell with the composition of step (a), whereby a state of the insect cell is modulated.

In another aspect, the invention features a method of modulating a state of an animal cell, the method comprising: (a) providing a composition comprising a plurality of ADAS, wherein the ADAS are derived from a parent bacterium having a reduction in the level or activity of a cell division topological specificity factor and wherein the composition is substantially free of viable bacterial cells; and (b) contacting the animal cell with the composition of step (a), whereby a state of the animal cell is modulated.

In another aspect, the invention features a method of modulating a state of a plant cell, the method comprising: (a) providing a composition comprising a plurality of ADAS, wherein the ADAS are derived from a parent bacterium having a reduction in the level or activity of a cell division topological specificity factor and wherein the composition is substantially free of viable bacterial cells; and (b) contacting the plant cell with the composition of step (a), whereby a state of the plant cell is modulated.

In another aspect, the invention features a method of modulating a state of an insect cell, the method comprising: (a) providing a composition comprising a plurality of ADAS, wherein the ADAS are derived from a parent bacterium having a reduction in the level or activity of a cell division topological specificity factor and wherein the composition is substantially free of viable bacterial cells; and (b) contacting the insect cell with the composition of step (a), whereby a state of the insect cell is modulated.

The modulating may be any observable change in the state (e.g., the transcriptome, proteome, epigenome, biological effect, or health or disease state) of the cell (e.g., an animal, plant, or insect cell) as measured using techniques and methods known in the art for such a measurement, e.g., methods to measure the level or expression of a protein, a transcript, an epigenetic mark, or to measure the increase or reduction of activity of a biological pathway. In some embodiments, modulating the state of the cell involves increasing a parameter (e.g., the level or expression of a protein, a transcript, or activity of a biological pathway) of the cell. In other embodiments, modulating the state of involves decreasing a parameter (e.g., the level or expression of a protein, a transcript, or activity of a biological pathway) of the cell.

### D. Methods of Treating an Animal, a Plant, or an Insect

In some aspects, the invention features a method of treating an animal in need thereof, the method comprising (a) providing a composition comprising a plurality of highly active achromosomal dynamic active systems (ADAS), wherein the ADAS have an initial ATP concentration of at least 1.25 mM and wherein the composition is substantially free of viable bacterial cells; and (b) contacting the animal with an effective amount of the composition of step (a), thereby treating the animal.

In other aspects, the invention features a method of treating an animal in need thereof, the method comprising: (a) providing a composition comprising a plurality of ADAS, wherein the ADAS are derived from a parent bacterium having a reduction in the level or activity of a cell division topological specificity factor and wherein the composition is substantially free of viable bacterial cells; and (b) contacting the animal with an effective amount of the composition of step (a), thereby treating the animal.

The animal in need of treatment may have a disease, e.g., a cancer. In some embodiments, the ADAS carries a chemotherapy cargo or an immunotherapy cargo.

In some aspects, the invention features a method of treating a plant in need thereof, the method comprising (a) providing a composition comprising a plurality of highly active achromosomal dynamic active systems (ADAS), wherein the ADAS have an initial ATP concentration of at least 1.25 mM and wherein the composition is substantially free of viable bacterial cells; and (b) contacting the plant or a pest (e.g., an insect pest) thereof with an effective amount of the composition of step (a), thereby treating the plant.

In other aspects, the invention features a method of treating an plant in need thereof, the method comprising: (a) providing a composition comprising a plurality of ADAS, wherein the ADAS are derived from a parent bacterium having a reduction in the level or activity of a cell division topological specificity factor and wherein the composition is substantially free of viable bacterial cells; and (b) contacting the plant or a pest (e.g., an insect pest) thereof with an effective amount of the composition of step (a), thereby treating the plant.

In an additional aspect, the invention provides methods of modulating a target cell. The target cell can be any cell, including an animal cell (e.g., including humans and non-human animals, including farm or domestic animals, pests), a plant cell (including from a crop or a pest), a fungal cell, or a bacterial cell. The cell may be isolated, e.g., in vitro or, in other embodiments, within an organism, in vivo. These methods entail providing an ADAS provided by the invention or a composition provided by the invention with access to the target cell, in an effective amount. The access to the target cell may either be direct, e.g., where the target cell is modulated directly by the ADAS, such as by proximate secretion of some agent proximate to the target cell or injection of the agent into the target cell, or indirect. The indirect modulation of the target cell can be by targeting a different cell, for example, by modulating a cell adjacent to the target cell, which adjacent cell may be commensal or pathogenic to the target cell. The adjacent cell, like the target cell may be either in vitro or in vivo—i.e., in an organism, which may be commensal or pathogenic. These methods are collectively “methods of use provided by the invention” and the like. In a related aspect, the invention provides target uses of the ADAS and compositions provided by the invention, consonant with the methods of use provided by the invention.

For example, in some embodiments, the invention provides method of modulating a state of an animal cell, by providing an effective amount of an ADAS provided by the invention or composition provided by the invention access to the animal cell. In certain embodiments, the ADAS or composition is provided access to the animal cell in vivo, in an animal, such as a mammal, such as a human. In some embodiments, the animal cell is exposed to bacteria in a healthy animal. In more particular embodiments, the animal cell is lung epithelium, an immune cell, skin cell, oral epithelial cell, gut epithelial cell, reproductive tract epithelial cell, or urinary tract cell. In still more particular embodiments, the animal cell is a gut epithelial cell, such as a gut epithelial cell from a human subject with an inflammatory bowel disease, such as Crohn's disease or colitis. In yet more particular embodiments, the animal cell is a gut epithelial cell from a subject with an inflammatory bowel disease, and the ADAS comprises a bacterial secretion system and a cargo comprising an anti-inflammatory agent.

In other embodiments the animal cell is exposed to bacteria in a diseased state. In certain embodiments, the animal cell is pathogenic, such as a tumor. In other embodiments, the animal cell is exposed to bacteria in a diseased state, such as a wound, an ulcer, a tumor, or an inflammatory disorder

In certain embodiments, the ADAS is derived from an animal commensal parental strain. In other embodiments, the ADAS is derived from animal pathogenic parental strain.

In certain particular embodiments, the animal cell is contacted to an effective amount of an ADAS comprising a T3/4SS or T6SS and a cargo, wherein the cargo is delivered into the animal cell. In some particular embodiments, the animal cell is provided access to an effective amount of an ADAS comprising a cargo and a secretion system, wherein the cargo is secreted extracellularly and contacts the animal cell.

In some embodiments, the state of the animal cell is modulated by providing an effective amount of an ADAS provided by the invention or a composition provided by the invention with access to a bacterial or fungal cell in the vicinity of the animal cell. That is, these methods entail indirectly modulating the state of the animal cell. In certain embodiments, the bacterial or fungal cell is pathogenic. In more particular embodiments, the fitness of the pathogenic bacterial or fungal cell is reduced. In other certain embodiments, the bacterial or fungal cell is commensal. In more particular embodiments, the fitness of the commensal bacterial or fungal cell is increased. In still more particular embodiments, the fitness of the commensal bacterial or fungal strain is increased via reduction in fitness of number of a competing bacteria or fungi, which may be neutral, commensal, or pathogenic.

In certain particular embodiments, the bacterial or fungal cell in the vicinity of the animal cell is contacted to an effective amount of ADAS comprising a T3/4SS or T6SS and a cargo, wherein the cargo is delivered into the bacterial or fungal cell. In other particular embodiments, the bacterial or fungal cell in the vicinity of the animal cell is provided access to an effective amount of ADAS secreting cargo extracellularly that contacts the bacterial or fungal cell.

In certain embodiments, the ADAS is derived from a parental strain that is a competitor of the bacterial or fungal cell. In other embodiments, the ADAS is derived from a from a parental strain that is mutualistic bacteria of the bacterial or fungal cell.

As will be appreciated, the various method of use provided by the invention that modulate the state of an animal cell can readily be adapted to corresponding methods for modulating the state of a plant, fungal, or bacterial cell. For illustrative purposes, methods for modulating the cell of a plant or fungal cell will be recited more particularly.

Accordingly, in a related aspect the invention provide methods of modulating a state of a plant or fungal cell by providing an effective amount of an ADAS provided by the invention or composition provided by the invention access to: a) the plant or fungal cell, b) an adjacent bacterial or adjacent fungal cell in the vicinity of the plant or fungal cell, or c) an insect or nematode cell in the vicinity of the plant or fungal cell.

In certain embodiments, the ADAS is provided access to the plant cell in planta, e.g., in a crop plant such as row crops, including corn, wheat, soybean, and rice, and vegetable crops including solanaceae, such as tomatoes and peppers; cucurbits, such as melons and cucumbers; brassicas, such as cabbages and broccoli; leafy greens, such as kale and lettuce; roots and tubers, such as potatoes and carrots; large seeded vegetables, such as beans and corn; and mushrooms. In some embodiments, the plant or fungal cell is exposed to bacteria in a healthy plant or fungus. In other embodiments, the plant or fungal cell is exposed to bacteria in a diseased state.

In certain embodiments, the plant or fungal cell is dividing, such as a meristem cell, or is pathogenic, such as a tumor. In some embodiments, the plant or fungal cell is exposed to bacteria in a diseased state, such as a wound, or wherein the plant or fungal cell is not part of a human foodstuff.

For certain embodiments the ADAS is derived from a commensal parental strain. In other embodiments, the ADAS is derived from a plant or fungal pathogenic parental strain.

In some embodiments, the ADAS comprises an T3/4SS or T6SS and a cargo, and the cargo is delivered into the plant or fungal cell. In other embodiments, the plant or fungal cell is provided access to an effective amount of an ADAS comprising a bacterial secretion system and a cargo, wherein the bacterial secretion system secretes the cargo extracellularly, thereby contacting the plant or fungal cell with the cargo.

In some embodiments, the methods entail providing an effective amount of an ADAS or composition access to the adjacent bacterial or adjacent fungal cell in the vicinity of the plant or fungal cell. In more particular embodiments, the adjacent bacterial or adjacent fungal cell is pathogenic, optionally wherein the fitness of the pathogenic adjacent bacterial or adjacent fungal cell is reduced. In other more particular embodiments, the adjacent bacterial or adjacent fungal cell is commensal, optionally wherein the fitness of the commensal adjacent bacterial or adjacent fungal cell is increased. In still more particular embodiments, the fitness is increased via reduction of a competing bacteria or competing fungi, which may be neutral, commensal, or pathogenic.

In some embodiments, the adjacent bacterial or adjacent fungal cell is contacted with an effective amount of ADAS comprising a T3/4SS or T6SS and a cargo, wherein the cargo is delivered into the adjacent bacterial or adjacent fungal cell.

In other embodiments, the adjacent bacterial or adjacent fungal cell is provided access to an effective amount of ADAS comprising a bacterial secretion system and a cargo, wherein the bacterial secretion system secretes the cargo extracellularly, thereby contacting the adjacent bacterial or adjacent fungal cell with the cargo.

In some embodiments, the ADAS is derived from a parental strain that is a competitor of the adjacent bacterial or adjacent fungal cells. In other embodiments, the ADAS is derived from a parental strain that is a mutualistic bacterium of the adjacent bacterial or adjacent fungal cell.

In certain embodiments, the methods include providing an effective amount of the ADAS or composition access to an insect or nematode cell in the vicinity of the plant or fungus. In more particular embodiments, the insect or nematode is pathogenic. In still more particular embodiments, the fitness of the pathogenic insect or nematode cell is reduced. In yet more particular embodiments, the fitness of the pathogenic insect or nematode cell is reduced via modulation of symbiotes in the insect or nematode cell. In other particular embodiments, the insect or nematode is commensal. In more particular embodiments, the fitness of the commensal insect or nematode cell is increased. In still more particular embodiments, the fitness is increased via reduction of a competing bacteria or fungi, which may be neutral, commensal, or pathogenic.

In yet another aspect, the invention provide methods of removing one or more undesirable materials from an environment comprising contacting the environment with an effective amount of an ADAS provided by the invention or composition provided by the invention, wherein the ADAS comprises one or more molecules (such as proteins, polymers, nanoparticles, binding agents, or a combination thereof) that take up, chelate, or degrade the one or more undesirable materials. “Environments” are defined as targets that are not cells, such as the ocean, soil, superfund sites, skin, ponds, the gut lumen, and food in a container.

In certain embodiments, the undesirable material includes a heavy metal, such as mercury, and the ADAS includes one or more molecules (such as proteins, polymers, nanoparticles, binding agents, or a combination thereof) that bind heavy metals, such as MerR for mercury. In some embodiments, the undesirable material includes a plastic, such as PET, and the ADAS includes one or more plastic degrading enzymes, such as PETase. In certain embodiments, the the undesirable material comprises one or more small organic molecules and the ADAS comprise one or more enzymes capable of metabolizing said one or more small organic molecules.

### E. RNA Delivery Methods

In another aspect, the invention provides a composition containing a bacterium or ADAS provided by the invention, wherein the bacterium or ADAS includes a T4SS, an RNA binding protein cargo, and an RNA cargo that is bound by the RNA binding protein and is suitable for delivery into a target cell through the T4SS. In certain embodiments, the RNA binding protein is a Cas9 fused to VirE2 and VirF, the RNA cargo is a guide RNA, and, optionally, the T4SS is the Ti system from Agrobacterium. In other embodiments, the RNA binding protein is p19 from Carnation Italian Ringspot Virus fused to VirE2 or VirF, the RNA cargo is an siRNA, and optionally wherein the T4SS is the Ti system from Agrobacterium. In a related aspect, the invention provides methods of making these particular compositions, such methods entailing transfecting a plasmid containing the Cas9 fused to VirE2 and VirF and RNA cargo into an Agrobacterium cell.

In a further related aspect, the invention provides methods for delivering RNA to a plant cell or animal cell comprising contacting said plant cell or animal cell with a bacterium or ADAS, wherein the bacteria or ADAS comprises a T4SS, an RNA binding protein cargo, and an RNA cargo, wherein the RNA is delivered to the plant cell or animal cell. In more particular embodiments, the RNA-binding protein cargo is also delivered to the plant cell or animal cell.

### EXAMPLES

Table of Contents

| | |
|---|---|
| Example 1 | Production of ADAS by genetic manipulation |
| Example 2 | Purification of ADAS |
| Example 3 | Characterization of ADAS |
| Example 4 | Measuring ADAS activity |
| Example 5 | ADAS generated from ΔminCDE parents are highly active |
| Example 6 | ADAS with membrane displayed proteins |
| Example 7 | Cargo Production and Delivery by ADAS |
| Example 8 | Loading of E. coli ADAS with RNA cargo |
| Example 9 | Delivery of E. coli ADAS to Lepidoptera |
| Example 10 | Storage of ADAS |
| Example 11 | Supplemental methods for characterization of ADAS |
| Example 12 | Supplemental methods for production of E. coli ADAS |
| Example 13 | Supplemental methods for the purification of ADAS derived from E. coli and efficiency |
| | measurement |
| Example 14 | Preparation of large E. coli ADAS |
| Example 15 | Supplemental methods for producing auxotrophic ADAS-producing bacterial strains to |
| | increase purity |
| Example 16 | Supplemental methods for measuring ADAS activity |
| Example 17 | Supplemental methods for storage of ADAS derived from E. coli |
| Example 18 | Assessing similarity of large ADAS to parent bacteria |
| Example 19 | Supplementary methods for demonstrating higher purity of auxotrophic ADAS |
| Example 20 | Supplementary methods for determining whether ADAS with expressed ATP synthase |
| | are highly active |
| Example 21 | Assays for increased activity in ADAS with suppression of ATP synthase ε components |
| Example 22 | Highly active ADAS with modified glycolysis pathways |
| Example 23 | Assays for increased activity in ADAS synthesized in specialized culture conditions |
| Example 24 | Assays for increased activity in ADAS with removed flagella and other non-essential |
| | features |
| Example 25 | Assays for increased activity in ADAS with photosynthetic capabilities |
| Example 26 | Supplemental methods for creating E. coli ADAS with stable nucleic acid |
| | and protein payloads |
| Example 27 | Increasing cargo stability by removal of ribonucleases from E. coli ADAS |
| Example 28 | Synthesis of a B. subtilis ADAS with a Tat signal peptide |
| Example 29 | Immunomodulatory ADAS for tumor suppression |
| Example 30 | Synthesis of Pseudomonas putida ADAS and highly active ADAS |
| Example 31 | T6SS-expressing ADAS as an antimicrobial and a plant protectant |
| Example 32 | Serratia marcescens ADAS as a model antifungal ADAS |
| Example 33 | Delivering proteins to human gut epithelial cells via overexpressed T3SS |
| Example 34 | Supplemental methods for targeted delivery of E. coli ADAS with T3SS systems |
| Example 35 | Synthesis of Xanthomonas citri ADAS for T3SS delivery to plants |
| Example 36 | Synthesis of Agrobacterium ADAS as a plant transformation agent using T4SS |
| Example 37 | Measuring ADAS secretion efficiency |
| Example 38 | Non-rod-shaped nanoparticle-containing ADAS (Magnetospirillum magneticum) |
| Example 39 | Environmentally responsive ADAS |
| Example 40 | Heavy metal-scavenging ADAS from E. coli and extremophiles |
| Example 41 | Lactose uptake and metabolism by E. coli ADAS |
| Example 42 | PETase-expressing E. coli ADAS for degradation of plastic |
| Example 43 | RNA secretion via a T4 secretion system |

### Example 1: Production of ADAS by Genetic Manipulation

ADAS may be produced from parent bacterial cells by several means. In this example, ADAS are produced by disruption of one or more genes involved in regulating parent cell partitioning functions, i.e., disruption of a z-ring inhibition protein (e.g., AminC or AminD) or disruption of z-ring inhibition proteins and a cell division topological specificity factor (e.g., AminCDE). This example details genetic means of creating ADAS-producing strains via disruption of the min operon or over-expression of the septum machinery component FtsZ.

### A. Production of ADAS Via Min Mutations

To disrupt the min operon, Lambda-RED recombineering methodology was adopted according to protocols laid out in Datsenko and Wanner, PNAS, 97 (12): 6640-6645, 2000. Strains for engineering and containing the plasmids for the Lambda-RED system were acquired from the Coli Genetic Stock Center (CGSC) at Yale University. Briefly, primers were designed to nonpolarly delete the coding sequences of E. coli minC (generating the parent bacterial strain MACH061), minD (generating the parent bacterial strain MACH062), or the entire minCDE operon (generating the parent bacterial strain MACH060) by encoding approximately 40 base pairs of genomic homology into 5′ ends of primers. The 3′ ends of these primers are homologous to plasmids pKD3 and pKD4 of the Lambda-RED system, which provide antibiotic markers that were used to select for parent bacterial strains inheriting the target mutations. Primer sequences used for deletion are provided in Table 1. After performing standard PCR using the primers with pKD3 as a DNA template, the purified amplicon was transformed via electroporation into bacteria prepared with pKD46, the plasmid containing the phage-derived Lambda-RED homologous recombination system, according to the methods of Datsenko and Wanner, PNAS, 97 (12): 6640-6645, 2000. Transformants were selected on LB agar with 35 μg/mL chloramphenicol. These resulting colonies were confirmed to have the genetic disruption (i.e., AminC, AminD, or AminCDE) using standard allele-specific PCR. Strain genotypes are provided in Table 2.

TABLE 1
Primers

| Primer Name | Description | Sequence |
|---|---|---|
| oAF75 | KO MinCDE FWD | SEQ ID NO: 10 |
| oAF76 | KO MinCDE REV | SEQ ID NO: 11 |
| oAF77 | KO MinC FWD | SEQ ID NO: 12 |
| oAF78 | KO MinC REV | SEQ ID NO: 13 |
| oAF79 | KO MinD FWD | SEQ ID NO: 14 |
| oAF80 | KO MinD REV | SEQ ID NO: 15 |
| oAF167 | KO SalTy MinCDE | SEQ ID NO: 16 |
| | FWD | |
| oAF168 | KO SalTy MinCDE REV | SEQ ID NO: 17 |

TABLE 2
Strains

| Name | Description | Genotype | Alias |
|---|---|---|---|
| MACH002 | _{X}1488 pGreenTIR | F-, purE41, glnV42(AS), λ-, serC53, minB- | Yale |
| | | 2, his-53, xyl-14, metB65, cycA1, hsdR2, tte- | University Coli |
| | | 1, cycB2, ilv-277 | Genetic Stock |
| | | | Center |
| | | | (CGSC) |
| | | | 14165 |
| MACH009 | BW25113 | Δ(araD-araB)567, ΔlacZ4787(::rrnB-3), | CGSC 7636 |
| | | λ-, rph-1, Δ(rhaD-rhaB)568, hsdR514 | |
| MACH060 | MACH009 ΔminCDE::CamR | BW25113 ΔminCDE::CamR | This work |
| MACH061 | MACH009 ΔminC::CamR | BW25113 ΔminC::CamR | This work |
| MACH062 | MACH009 ΔminD::CamR | BW25113 ΔminD::CamR | This work |
| MACH065 | MG1655 | F-, λ-, rph-1 | CGSC 7740 |
| MACH124 | MACH060 pGFP | BW25113 ΔminCDE::CamR pGFP | This work |
| MACH151 | TOP 10 pFtsZ | TOP10 pFtsZ | This work |
| MACH178 | MACH009 pFtsZ | BW25113 pFtsZ | This work |
| MACH198 | MACH060 pSTING | BW25113 ΔminCDE::CamR pSTING | This work |
| MACH199 | Vibrio parahaemolyticus | Vibrio parahaemolyticus EB101 pFtsZ | American |
| | pFtsZ | | Type Culture |
| | | | Collection |
| | | | (ATCC) 17802 |
| MACH200 | MACH065 ΔminCDE::CamR | MG1655 ΔminCDE::CamR | This work |
| MACH269 | SalTy ΔminCDE pHilA-KanR | Salmonella typhimurium LT2 | ATCC 23564 |
| | | ΔminCDE::CamR pHilA-KanR | |
| MACH270 | SalTy ΔminCDE pHilA-AmpR | Salmonella typhimurium LT2 | This work |
| | | ΔminCDE::CamR pHilA-AmpR | |
| MACH284 | MACH060 pNeae-NB2 | BW25113 ΔminCDE::CamR pNeae-NB2 | This work |
| MACH300 | HT115(DE3) | F-, mcrA, mcrB, IN(rrnD-rrnE)1, | CGSC |
| | | rnc14::Tn10(DE3 lysogen: lacUV5 promoter- | HT115(DE3) |
| | | T7 polymerase | |
| MACH301 | MACH300 pRNAi | MACH300 ΔminCDE::CamR | This work |
| MACH320 | MACH060 pT3SS pVirB | BW25113 ΔminCDE::CamR pT3SS pVirB | This work |
| MACH434 | MACH060 pOspB-TEM1 | BW25113 ΔminCDE::CamR pOspB-TEM1 | This work |
| MACH435 | MACH060 pT3SS pVirB | BW25113 ΔminCDE::CamR pT3SS pVirB | This work |
| | pOspB-TEM1 | pOspB-TEM1 | |
| MACH556 | MACH061 pGFP | BW25113 ΔminC::CamR pGFP | This work |
| MACH557 | MACH062 pGFP | BW25113 ΔminD::CamR pGFP | This work |

B. Production of ADAS by Overexpression of ftsZ

To create ADAS from the overexpression of septum machinery, we constructed a plasmid that drives expression of the FtsZ Z-ring protein from wild-type E. coli. In brief, a strong ribosome binding site and the coding sequence for the E. coli FtsZ protein were de novo optimized using computational tools from De Novo DNA. This translational unit was ordered for de novo DNA synthesis from Integrated DNA Technologies (IDT™) and cloned into a backbone using standard cloning techniques. The resulting plasmid, pFtsZ (Table 3), features a TetR repressor, a TetA promoter that is repressed by the TetR protein, a kanamycin resistance marker, and a pMB1 origin of replication. When transformed into a compatible bacterium, pFtsz can be induced to overproduce the FtsZ protein via addition of anhydrotetracycline to the culture. This protein is then capable of forming spontaneous protofilaments, which cause asymmetric division of parent bacterial cells and, thereby, ADAS production.

TABLE 3
Plasmids

| Plasmid Name | Description | Sequence | Reference |
|---|---|---|---|
| pFtsZ | pMB1 KanR TetR-PtetA-FtsZ | SEQ ID NO: | This work |
| | | 18 | |
| pGFP | pMB1 KanR TetR-PtetA-sfGFP | SEQ ID NO: | This work |
| | | 19 | |
| pGreen TIR | pUC AmpR GFP | | Miller and |
| | | | Lindow, |
| | | | Gene, |
| | | | 191(2): 149- |
| | | | 53, 1997. |
| pHilA-CloDF | CloDF13 AmpR TetR-PtetA-HilA | SEQ ID NO: | This work |
| | | 20 | |
| pHilA-pMB1 | pMB1 AmpR TetR-PtetA-HilA | SEQ ID NO: | This work |
| | | 21 | |
| pNeae-NB2 | CloDF13 KanR TetR-PtetA-Neae-NB2 | SEQ ID NO: | This work |
| | | 22 | |
| pOspB-TEM1 | ColE1 AmpR Plac-OspB-TEM1 | | Reeves et. |
| | | | al., ACS Syn |
| | | | Bio, 4; 644- |
| | | | 654, 2015 |
| pRNAi | pMB1 AmpR T7- P. xylostella chy1-T7rev | SEQ ID NO: | This work |
| | | 23 | |
| pSTING | pMB1 AmpR TetR-PtetA-DacA | SEQ ID NO: | This work |
| | | 24 | |
| pT3SS | RK2 KanR Shigella flexneri | | Mou et |
| | minimum type 3 secretion system | | al., PNAS, |
| | | | 115(25), |
| | | | 6452-6457, |
| | | | 2018 |
| pVirB | pSC101 SpecR Plac-VirB | | Reeves et. |
| | | | al., ACS Syn |
| | | | Bio, 4; 644- |
| | | | 654, 2015 |

### Example 2: Purification of ADAS

This example describes methods of purifying populations of ADAS from a culture of an ADAS-producing bacterial parent strain. This method may be employed to purify any of the ADAS-producing strains described herein, including the strains of Example 1 or Table 3. Purification separates ADAS from viable parent bacterial cells, which are larger and contain a genome. ADAS were purified from high cell density cultures of ADAS-producing strains via combinations of 1) low speed centrifugation, 2) selective outgrowth, and 3) buffer exchange/concentration. Low-speed centrifugation procedures were used to selectively deplete viable parent bacterial cells and large cellular debris, while enriching ADAS in a mixed suspension. Selective outgrowth procedures were used to reduce the number of viable parent bacterial cells present in the sample via the addition of compounds that are directly anti-microbial (i.e., toxic to cells having a microbial genome) and/or compounds that enhance viable cell sedimentation via low speed centrifugation. Buffer exchange/concentration procedures were used to transition ADAS from larger volumes of bacterial culture media into smaller volumes of 1×PBS while removing culture additives and cellular debris.

### A. ADAS Purification

ADAS-producing strains were generated using the molecular cloning procedures described in Example 1, then cultured to high cell density in culture medium. Cultures may be scaled up, e.g., from 1 mL to 1000 mL or more culture medium.

Cultures were transferred to centrifuge tubes and subjected to a low-speed centrifugation procedure aimed at pelleting intact cells and large cell debris while maintaining ADAS in the supernatant. Low-speed centrifugation procedures were performed either at 4° C. or at room temperature. In some instances, the low-speed centrifugation procedure was a sequence of sequential 10-minute spins at 1,000×g, 2,000×g, 3,000×g, and 4,000×g performed on an Allegra® X14R benchtop centrifuge (Beckman Coulter) or an Eppendorf™ 5424 R benchtop centrifuge (Fisher Scientific). In some instances, the low-speed centrifugation procedure consisted of sequential 2,000×g spins for 20 minutes at 4° C. in which the supernatant of the first spin was decanted into a sterile centrifuge bottle prior to the second spin. In some instances, the low-speed centrifugation procedure was a single 40-minute spin at 4,000×g in a Sorvall™ Lynx 6000 Superspeed Centrifuge (Thermo Scientific™) in which the rate of rotor acceleration was set to the lowest possible setting.

Following low-speed centrifugation, culture supernatants were decanted into sterile culture tubes and subjected to a selective outgrowth process. In some instances, concentrated antibiotic solutions (e.g., ceftriaxone, kanamycin, carbenicillin, gentamicin, and/or ciprofloxacin) or other concentrated chemical solutions (e.g., sodium chloride, sodium hydroxide, M hydrochloric acid, glucose, casamino acids, and/or D-amino acids) were added directly to the culture supernatants. In other cases, the culture supernatants were pelleted via high-speed centrifugation for 5 to 60 minutes at 10,000×g to 20,000×g and the pellets were resuspended in fresh culture media containing concentrations of antibiotics or other chemical solutions that were inhibitory to viable cells. Selective outgrowth was performed by incubating ADAS at 4° C. to 42° C. for 1 to 3 hours with agitation at 250 rpm. ADAS were then transferred to sterile centrifuge tubes and subjected to an additional round of low-speed centrifugation for 15 minutes at 4° C., 4,000×g.

Following selective outgrowth and low-speed centrifugation, supernatants were subjected to buffer exchange/concentration procedures. In some instances, this was carried out by passing supernatants over a 0.2 μm asymmetrical polyethersulfone (aPES) membrane filter (Thermo Fisher) followed by 1 to 9 volumes of 1×PBS. In other cases, ADAS were pelleted via centrifugation at 10,000×g to 20,000×g for 5 to 60 minutes, washed in 1 to 9 volumes of 1×PBS, pelleted again, and resuspended in 1× PBS at 1 to 100,000× concentration from the starting culture volume.

B. ADAS Purification from Auxotrophic ADAS-Producing Parental Strains

ADAS-producing parental strains that are auxotrophic, i.e., are unable to synthesize an organic compound required for growth, are useful for the manufacturing of ADAS. Such strains are able to grow only when the organic compound is provided. Auxotrophic parental strains may thus be selected against by storing or incubating an ADAS preparation in media lacking the organic compound, thus providing an additional method for reducing parental burden in the ADAS preparation.

Preparation of Auxotrophic E. coli Parental Bacteria

One auxotrophic ADAS-producing parental strain was acquired and a second strain was generated. MACH002 (Table 2) was acquired from the Yale University Coli Genetic Stock Center (CGSC) (strain CGSC14165). MACH002 is a histidine (his-53) and methionine (metB65) double-auxotrophic strain having a minB disruption that produces ADAS. To construct the second auxotrophic ADAS-producing parental strain, the pFtsZ plasmid described in Example 1 and Table 3 was transformed into the leucine auxotroph (leu-) Top10 E. coli strain and selected for with Kanamycin 50 μg/mL to create MACH151 (see Table 2 for full genotype). When anhydrotetracycline is added to the culture supernatant, TetR-repression of the TetA promoter is relieved, FtsZ protein is expressed, and parental bacteria produce ADAS (Example 1). E. coli ADAS were produced according to the methods of Example 2, but with histidine and methionine included in the media for MACH002 or leucine and anhydrotetracycline in the media for MACH151. The ADAS were purified using the methods in Example 2, and then stored in histidine-free and methionine-free media for MACH002 or leucine-free media for MACH151.

ADAS From Auxotrophic Parental Strains have Increased Purity

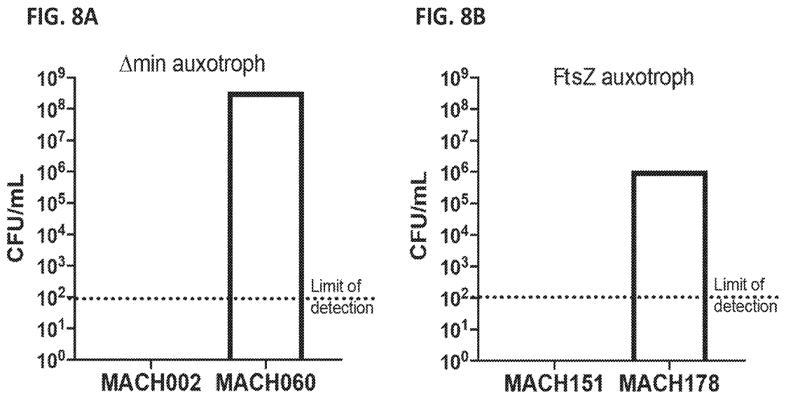

ADAS were generated from auxotrophic and non-auxotrophic parental strains. MACH060 (non-auxotrophic) and MACH002 (histidine methionine auxotroph) ADAS were prepared and purified by the process outlined in Example 2. MACH178 (non-auxotrophic FtsZ overexpressing line) and MACH151 (leucine auxotroph FtsZ-overexpressing line) ADAS were prepared and purified by the process outlined in Example 2, with a slight modification to the growth protocols: anhydrotetracycline was added to the cultures to a final concentration of 50 ng/mL during growth to induce ADAS production due to FtsZ expression. To measure the purity of ADAS preparations, 1 mL samples of supernatants were collected following the initial 4000×g sequential centrifugation step of the purification process in Example 2. These samples contained both ADAS and any parental bacterial cells that were not removed by sequential centrifugation. 100 μl aliquots were then plated on non-permissive media (M9+0.2% glucose) and incubated at 37° C. overnight. The next day, parental burden was enumerated by counting the number of CFU per plate (FIGS. 8A and 8B). While the ADAS preparations produced from wild-type MACH060 and MACH178 harbored significant parental burden, ADAS preparations from both the MACH002 and the MACH151 auxotroph demonstrated undetectable levels of parental bacteria. Thus, auxotrophic ADAS preparations produced by two different methods each have higher purity than ADAS preparations from non-auxotrophic ADAS. In some non-exhaustive embodiments, MACH002 shows at least a 106 less parental burden than MACH060; MACH151 shows at least 104 less parental burden than MACH178.

### Example 3: Characterization of ADAS

This example describes methods of characterizing purified populations of ADAS and/or unpurified ADAS-producing bacterial cultures using a variety of approaches, including electron microscopy, light microscopy and immunofluorescence, nanoparticle characterization, viable cell plating, immunoblotting, and flow cytometry.

### Scanning Electron Microscopy (SEM)

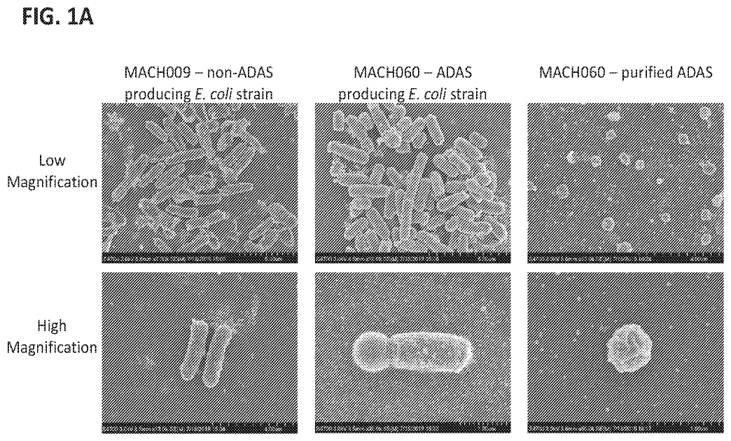

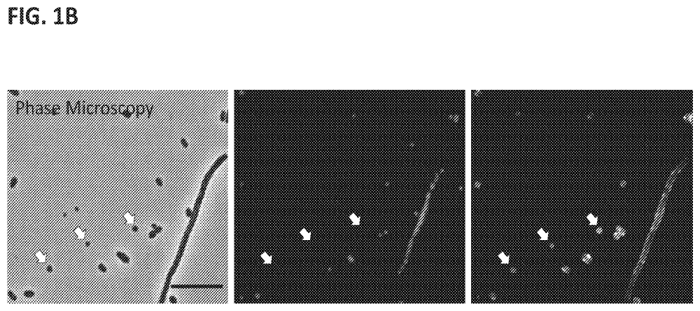

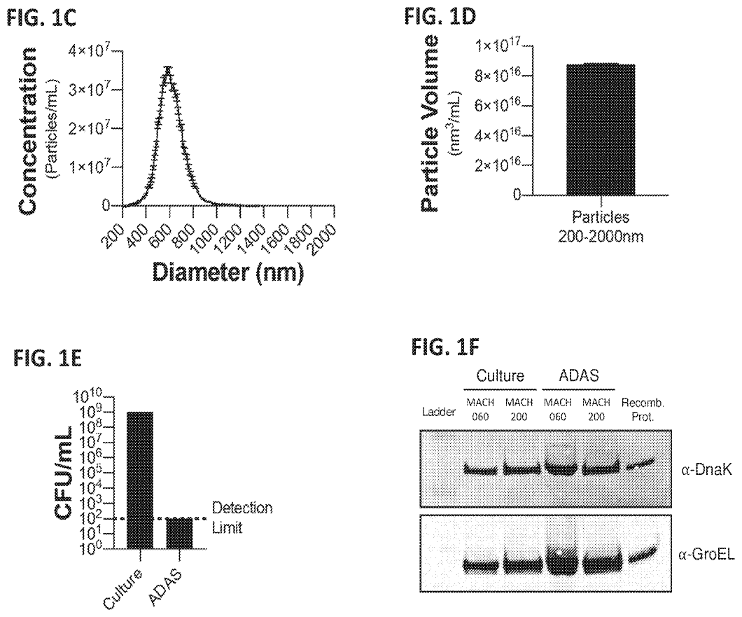

SEM was used to visualize the process of septation and confirm the existence of an ADAS population in various bacterial strains of interest. To prepare SEM samples, glass coverslips were placed in a 24 well plate and coated with 0.01% poly-lysine and dried. Then, strains of interest were generated using the molecular cloning procedures described in Example 1, cultured to high cell density and, in some cases, subjected to the ADAS purification procedures described in Example 2. The strains or purified ADAS of interest were suspended in PBS and transferred to the 24 well plate with poly-lysine coated glass coverslips and allowed to settle at room temperature for 30 minutes. The solution was carefully aspirated and replaced with SEM fixative (formaldehyde-glutaraldehyde 2.5% in 0.1M sodium cacodylate (sod cac) buffer. The next day the fixative was removed, samples were washed with water, and coverslips were removed from the 24 well plate and dried using critical point drying. After drying, the coverslips were mounted on carbon tape on Hitachi SEM mounts, sputter coated with a 10 nm film of platinum, and imaged using a Hitachi S-4700 field emission scanning electron microscope (FE-SEM). FIG. 1A shows representative images of MACH009 (Wild-type E. coli), MACH060 (E. coli with a minCDE deletion to enable ADAS production), and purified ADAS from MACH060 (see Table 2). The MACH009 wild type E. coli strain exhibits the normal phenotype of E. coli, in which all daughter cells are equally sized (FIG. 1A, left panel). The unpurified MACH060 strain shows a population with enhanced polydispersity in size and presence of ADAS particles, which are smaller and spherical (FIG. 1A, center panel). The purified MACH060 shows only the ADAS population (FIG. 1A, right panel). Using SEM as described above, ADAS produced from bacterial species in the genera Vibrio and Salmonella using different ADAS-producing techniques (overexpression of ftsZ and deletion of minCDE, respectively) can also be visualized in unpurified populations (data not shown). Furthermore, in unpurified images, aberrant septation events are prominent; aberrant septation is the event preceding ADAS production.

Light microscopy and immunofluorescence The MACH060 E. coli strain was generated using the molecular cloning procedures described in Example 1, then cultured to high cell density overnight. Separately, glass coverslip bottom 35 mm dishes from ibidi® were coated with a 0.01% poly-lysine solution for 5 min; the solution was aspirated and the dishes were allowed to dry. A dense suspension of MACH060 was placed on the coverslip bottom in 1 μL drops and allowed to almost dry. Then 1 mL of 2.8% paraformaldehyde/0.04% glutaraldehyde fixative was added for 30 min. Samples were washed 3× in PBS, permeabilized with 0.1% Triton™ X-100 (Sigma-Aldrich), washed 2× with PBS, further permeabilized with 10 μg/mL lysozyme, washed 2× with PBS, blocked with 1% donkey serum, washed 2× with PBS, stained with primary antibody (anti-E. coli LPS), washed, and stained with a secondary antibody bound to Alexa Fluor® 555 (Thermo Fisher). The dishes were counter-stained with DAPI for 10 min and washed 3× with PBS. The final dishes were imaged using a 100× oil-dipping objective lens on an Olympus IX83 inverted microscope (Olympus). Images were rendered using the publicly available software package Fiji. FIG. 1B shows representative images of phase microscopy (left panel) and fluorescence (center and right panels) at 100× showing that within the parent population, there are ADAS which have the LPS membrane stain, but lack a genome (no DAPI positive stain).

### Nanoparticle Characterization

An ADAS-producing strain of E. coli BW25113, MACH124, was generated using the molecular cloning procedures described in Example 1, then cultured to high cell density in 1 L of culture medium and subjected to the ADAS purification procedures described in Example 2. The purified population of ADAS was suspended in 1× PBS, diluted to a concentration between 107 and 109 particles per mL, and TWEEN® 20 (Sigma Aldrich) was added to a final concentration of 0.1% (v/v) to minimize particle aggregation. This suspension of ADAS was diluted 20-fold and loaded onto a TS2000 cartridge (Technology® Supplies Ltd.) and analyzed on a Spectradyne® nCS1™ Nanoparticle Analyzer. The nCS1™ measures both the size and concentration of individual particles in a suspension by applying a bias voltage across a constriction of defined size and monitoring changes in electrical resistance as a function of time (Fraikin et al., Nature Nanotechnology, 6 (5): 308-313, 2011). 24,392 particles within the range of 200 to 2,000 nm were measured. In other examples, the number of particles measured is between 10-100,000 particles. The resulting data were plotted as a cumulative size distribution (FIG. 1C), which reveals the concentration of particles between 200 and 2,000 nm in diameter in the suspension of ADAS purified from MACH124. The highest concentration of ADAS purified from MACH124 lies within the size range of 400 to 800 nm. Additionally, the data were exported as an integration range report, which enumerates the aggregate volume of 200 to 2,000 nm particles in the suspension of purified ADAS (FIG. 1D). Collectively, these data demonstrate that the concentration and size distribution of purified ADAS can be quantified via nanoparticle characterization methods. Additionally, quantification of small molecules (e.g., ATP—Examples 4 and 5), nucleic acids, or proteins (e.g., GFP-Examples 4 and 5) observed within a purified population of ADAS can be divided by the aggregate volume of ADAS or the number of particles present in the assay, enabling calculation of the average concentration of small molecules, nucleic acids, or proteins of interest within a purified population of ADAS.

### Viable Cell Plating

To determine the concentration of viable parent bacterial cells present before and after purification, the ADAS-producing culture and the purified ADAS population described in Example 3C were assayed via viable cell plating. Serial dilutions were prepared by repeatedly transferring 100 μL of either the ADAS-producing culture or the purified population of ADAS into 900 μL of 1× PBS. Then, 10 μL of each dilution was spotted on selective media and incubated at 37° C. to allow the growth of viable cells. After 24 hours, dilutions containing 1-100 colonies were counted, and this colony count was multiplied by the appropriate dilution factor to enumerate the number of colony forming units (CFU) per mL of sample (i.e., the concentration of viable cells present in each sample) (FIG. 1E). The MACH124 culture had >108 CFU/mL, consistent with a high-density culture of Escherichia coli; however, the number of viable cells present in the population of purified ADAS was less than 100 CFU/mL, the limit of detection for this assay. Thus, the concentration of viable cells present in a sample can be enumerated via viable cell plating, and the ADAS purification procedures described in Example 2 are sufficient to reduce the number of viable cells present in purified ADAS at least below 100 CFU/mL in some embodiments.

### Immunoblotting

To evaluate the ability to measure ADAS protein composition, we characterized the presence in ADAS of two housekeeping proteins, Dnak and GroEL, that are known to be present in parent E. coli bacterial cells. ADAS-producing strains of Escherichia coli BW25113 (MACH060) and MG1655 (MACH200) were generated using the molecular cloning procedures described in Example 1, then cultured to high cell density in 100 mL of culture medium and subjected to the ADAS purification procedures described in Example 2. In parallel, 5 mL aliquots of the ADAS-producing cultures were pelleted at 20,000×g and resuspended in a 5 mL of 1× PBS, then diluted 100-fold in 1× PBS. 4× NuPAGEIM LDS Sample Buffer (Thermo Fisher) was added to 100 μL aliquots of the diluted cultures and to the purified ADAS in order to lyse the samples. The various lysates were incubated at 85° C. for 2 minutes, then 40 μL of each sample was resolved on an SDS-polyacrylamide gel. A positive control, comprising 10 ng of purified recombinant E. coli GroEL protein (Abcam, ab51307) or recombinant DnaK protein (Abcam, ab51121), was also denatured in 1× LDS Sample Buffer at 85° C. for 2 minutes and resolved on the gel. Proteins were transferred to a nitrocellulose membrane that was blocked for 1 hour at room temperature in Intercept® (PBS) Blocking Buffer (LI-COR®) and incubated with a 1:1,000 dilution of an antibody targeting GroEL (Abcam, ab90522) or Dnak (Abcam, ab69617) at room temperature overnight. The blots were then washed in excess volumes of PBS+0.05% TWEEN® 20, incubated with anti-mouse (LI-COR®, 926-68070) and anti-rabbit (LI-COR®, 926-32211) antibodies conjugated to fluorescent dyes for 1 hour at room temperature, washed again in excess volumes of PBS+0.05% TWEEN® 20, and imaged on an Invitrogen™ iBrightIM Imager (Thermo Fisher). Specific bands corresponding to GroEL and Dnak were present in the lanes containing lysates from ADAS-producing cultures and in the lanes containing lysates from purified ADAS (FIG. 1F); thus, the protein content of ADAS can be detected via immunoblotting.

### Example 4: Measuring ADAS Activity

We tested the capacity of ADAS to perform cellular work and our ability to measure this work. We measured work capacity by examining the level of adenosine triphosphase (ATP) in ADAS and by assessing the ability of ADAS to transcribe and translate a target protein. The capacity of a cell to perform work is directly related to its level of ATP. ATP is the major energy carrier of prokaryotic and eukaryotic organisms, carrying chemical energy within the bonds between its three phosphate groups that is released when they are broken. ATP is required for essential cellular processes, like transcription, translation, transport, and metabolism.

### A. ATP Measurement

ADAS derived from MACH060 (BW25113 AminCDE; Table 2) parent bacteria were purified as described in Example 2 using selective outgrowth from a concentrated culture supernatant. The purified ADAS were subjected to nanoparticle characterization and viable cell plating procedures, as described in Example 3. The concentration of ADAS in the purified ADAS preparation was 5×10⁸ ADAS/mL, and the total volume of particles present was 3.2×10¹⁶ nm³/mL. CFU plating revealed that the concentration of viable cells (e.g., parent cells) present within the purified population of ADAS was at or below the limit of detection (100 CFU/mL).

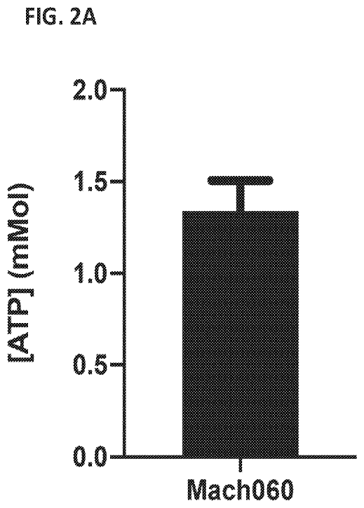

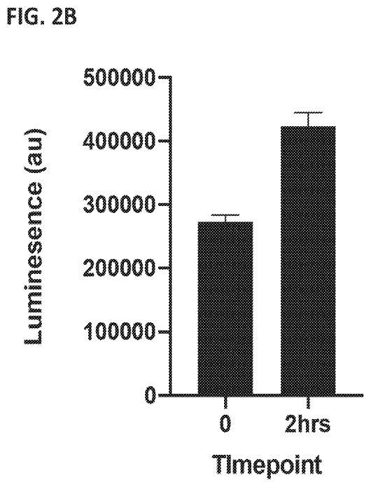

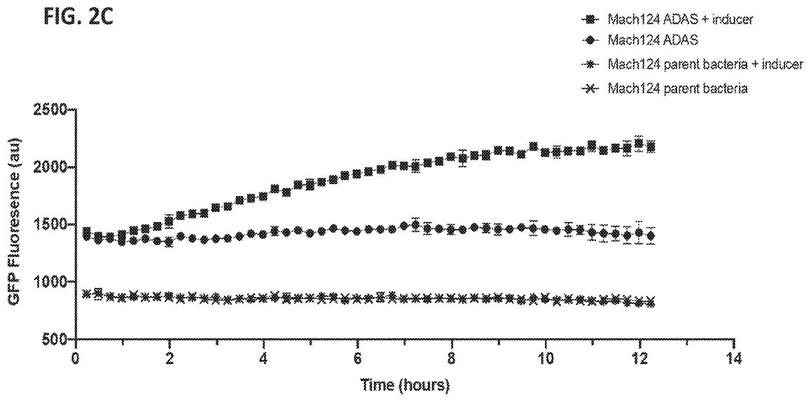

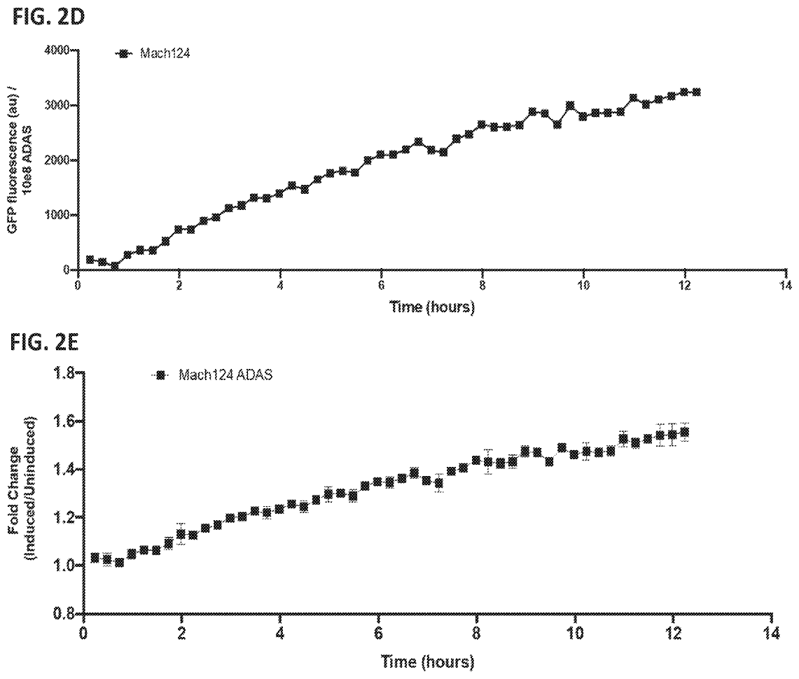

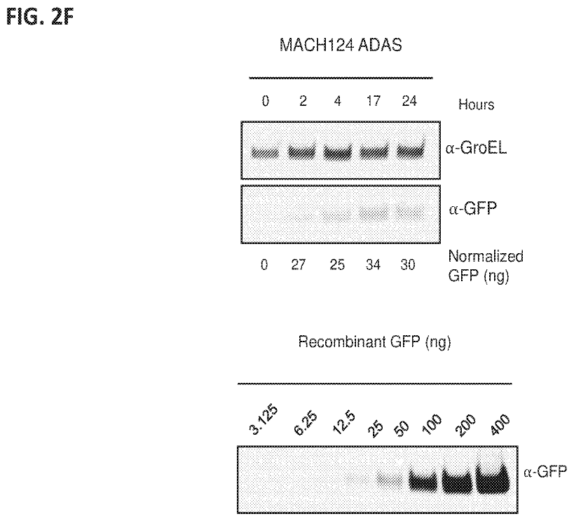

The ATP concentration present in the purified population of ADAS was measured in triplicate using the BacTiter-Glo™ Microbial Cell Viability Assay (Promega) following the manufacturer's instructions. Briefly, 100 μL of rehydrated assay buffer was added to wells containing 100 μL of purified ADAS or a defined mass of ATP, which served as a molecular standard. The luminescence signal from each well was recorded on a plate reader. The log-transformed luminescence signal for the defined masses of ATP were plotted against the log-transformed masses of ATP, and a line was fit to these data to generate a standard curve. Subsequently, the luminescence signal observed in wells containing purified ADAS was fit to this standard curve to calculate a mass of ATP present in the well (FIG. 2A). ATP levels generated by viable cell contaminants were below the limit of detection for the assay and were subtracted from the ADAS total as background. The mass of ATP present within each well of purified ADAS was divided by the aggregate particle volume of ADAS present within the well: this ratio represents the concentration of ATP present within a population of ADAS purified to <100 CFU/mL.

### B. ATP Generation

To determine the ability of ADAS to generate ATP in the absence of a genome, the ATP concentration from purified ADAS was measured over time. ADAS derived from MACH124 (BW25113 AminCDE; Table 2) were purified as indicated in Example 2 using selective outgrowth from a concentrated culture supernatant. Purified ADAS were supplemented with nutrient-rich media (10% Luria Broth) along with 50 μg/mL of the antibiotic carbenicillin. ADAS were split into two aliquots and tested in parallel; one aliquot was placed immediately into storage at 4° C., and the other aliquot was incubated in an Eppendorf ThermoMixer® at 37° C., 800 rpm for two hours. After the two-hour incubation, the ATP concentrations of both aliquots were measured as described above. Incubation for two hours resulted in a 54.9% increase in the amount of ATP, indicating that ADAS can generate ATP in the absence of a genome (FIG. 2B).

### C. Transcription and Translation of a Target Protein: GFP

To measure the capacity of ADAS to transcribe and translate a target gene, MACH060 (BW25113 AminCDE (Table 2)) was transformed with pGFP, a plasmid encoding green fluorescent protein (GFP). This sequence was codon-optimized for expression in E. coli and cloned into a plasmid containing the TetA promoter, the TetR repressor (which represses the TetA promoter), a pMB1 origin of replication, and a kanamycin resistance gene for antibiotic selection. ADAS were then derived from this strain (MACH124) and purified as indicated in Example 2, using selective outgrowth from a concentrated culture supernatant. The parental burden was determined to be at the limit of detection by CFU plating (<100 CFU/mL). To examine protein expression in ADAS, two methods were employed: (1) measuring GFP fluorescence over time using a plate reader, and (2) immunoblotting.

### (1) Plate Reader Method

Purified ADAS or parent bacteria were incubated in minimal growth media (Minimal salts (M9) media with 0.2% casamino acids, 0.2% glucose, and ceftriaxone at 100 ug/mL). Purified ADAS samples were supplemented with an antibiotic to prevent growth of any genome-containing parent bacteria. Additionally, for induced samples, the inducer, anhydrotetracycline, was added to the media to a final concentration of 100 ng/mL. Upon the addition of anhydrotetracycline, TetR repression of the TetA promoter was relieved and the GFP protein was expressed. GFP signal detection was measured using wavelengths of 479 nm and 520 nm for emission and excitation, respectively. FIGS. 2C-2E demonstrate an inducer-dependent increase in the transcription and translation of GFP, as measured by GFP fluorescence, in MACH124 ADAS over 12 hours. In the absence of the inducer (uninduced) there was no increase in the GFP signal. This finding shows that AminCDE ADAS are capable of transcribing and translating a target gene in an inducer-dependent manner. Further, FIG. 2C shows that the GFP signal generated by purified ADAS is not due to parent contamination, as the GFP signal from 100 CFU/mL of parent bacteria was significantly lower than that of the ADAS and did not increase over time.

### (2) Immunoblotting Method

MACH124 purified ADAS and an equal volume of Luria Broth supplemented with carbenicillin at 100 μg/mL and anhydrotetracycline (1000 ng/ml) inducer were aliquoted into sterile centrifuge tubes. The samples were incubated in an Eppendorf ThermoMixer® at 37° C., 800 rpm for 24 hours. After 24 hours, the ADAS were centrifuged at 20,000×g for 10 minutes at 4° C. The supernatant was removed and the pelleted ADAS were resuspended in lysis buffer (1x BugBuster™ Protein Extraction Reagent (MilliporeSigma™) with 1× NuPAGE™ LDS Sample Buffer (Thermo Fisher)) and heated at 85° C. for 2 minutes. An equal volume of lysed ADAS was then loaded into a 4-12% BisTris polyacrylamide gel. Proteins on the gel were resolved, transferred to a nitrocellulose membrane, and incubated in Intercept® (PBS) Blocking Buffer (LI-COR®) for 60 minutes at room temperature. The membrane was then incubated with primary antibody (Mouse anti-GroEL (Abcam, ab90522) at a 1:500 dilution and rabbit anti-GFP (Abcam, ab6556) at a 1:500 dilution) for 60 minutes at room temperature. Antibodies were resuspended in Intercept® PBS Blocking Buffer supplemented with 0.2% TWEEN® 20. The membrane was subsequently washed three times in 1× PBS+0.05% TWEEN® 20, followed by incubation in Intercept® PBS Blocking Buffer with 0.2% TWEEN® 20 supplemented with relevant secondary antibodies (Goat anti-Mouse 800 (Abcam, ab216772) and Goat anti-Rabbit 680 (Abcam, ab175773) both at a 1:5,000 dilution). The membrane was washed three times in 1× PBS+0.05% TWEEN® 20 and imaged on an Invitrogen™ iBright™ Imager (Thermo Fisher). The band intensity was quantitated via densitometry, and GFP intensity was expressed normalized against the loading control, GroEL. FIG. 2F demonstrates a time-dependent increase in the amount of GFP protein detected in the induced ADAS.

### Example 5: ADAS Generated from AminCDE Parents are Highly Active

ADAS purified from parent cells comprising the mutations AminC, AminD, or AminCDE were assayed via plate reader and immunoblotting to compare their abilities to transcribe and translate a GFP reporter gene. Unexpectedly, ADAS derived from AminCDE parent cells were found to have greater activity than ADAS derived from either AminC or AminD parent bacterial cells.

A. Purification of ADAS from AminCDE, AminC, or AminD Cultures

The ADAS-producing strains MACH124 (BW25113 AminCDE+pGFP), MACH556 (BW25113 AminC+pGFP), and MACH557 (BW25113 AminD+pGFP) (see Table 2) were cultured to high cell density in 1 L of culture medium and subjected to the ADAS purification procedures described in Example 2. The pGFP plasmid borne by all 3 of these strains is described in further detail in Table 3. The purified ADAS were subjected to the nanoparticle characterization and viable cell plating procedures described in Example 3. The concentration of ADAS was: MACH124=2.4×10⁹ ADAS/mL, MACH556=1.86×10⁹ ADAS/mL, and MACH557=1.98×10⁹ ADAS/mL. The viable parent bacterial cell burden present in the purified ADAS was found to be lower than the limit of detection by CFU plating (<100 CFU/mL).

### B. GFP Expression Kinetics Assayed Via Plate Reader

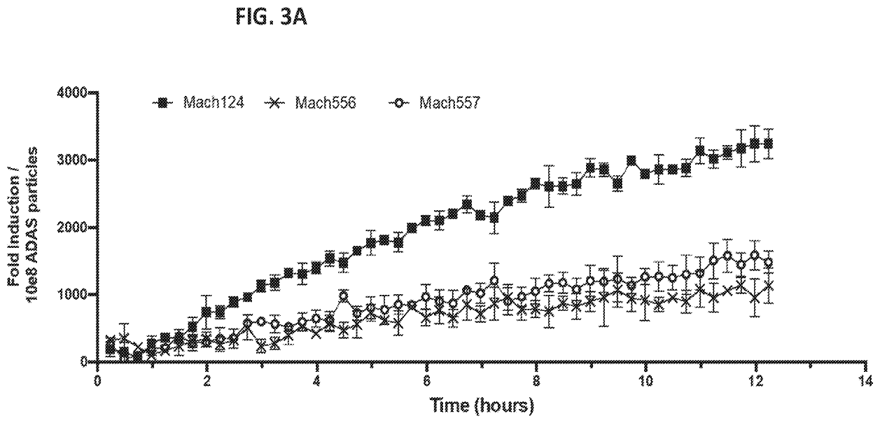

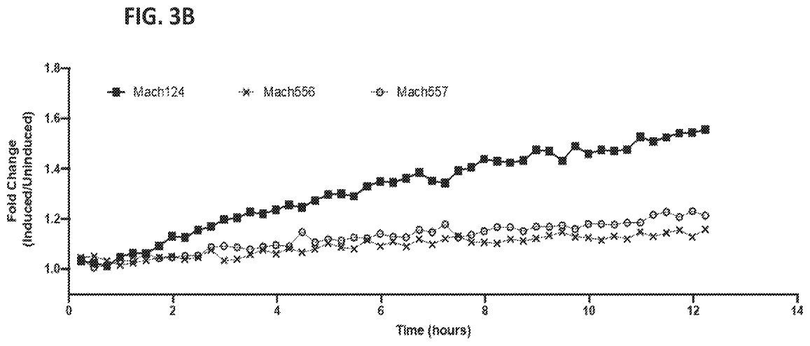

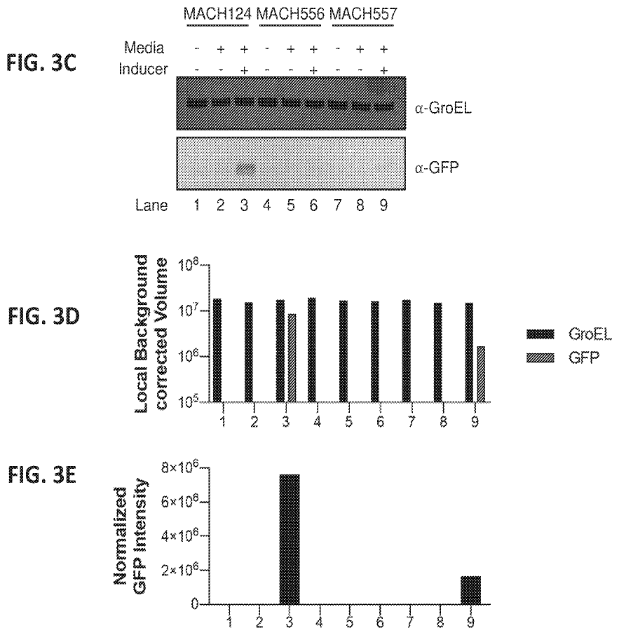

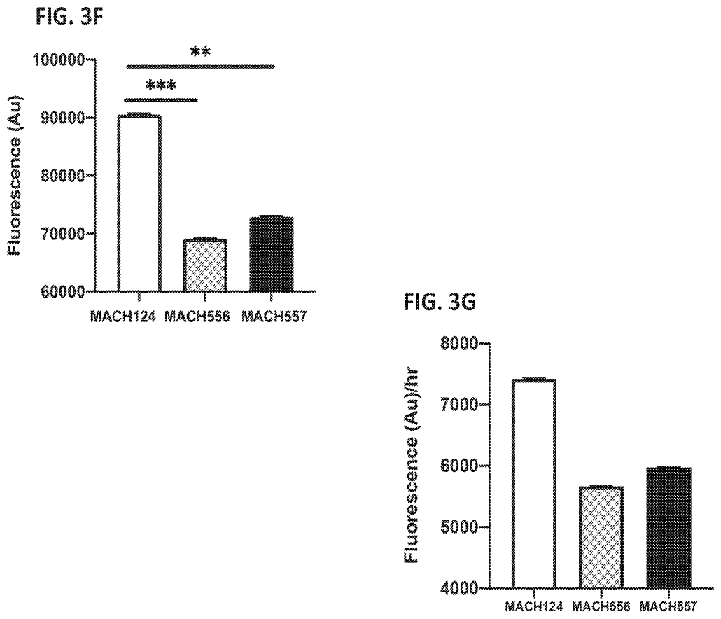

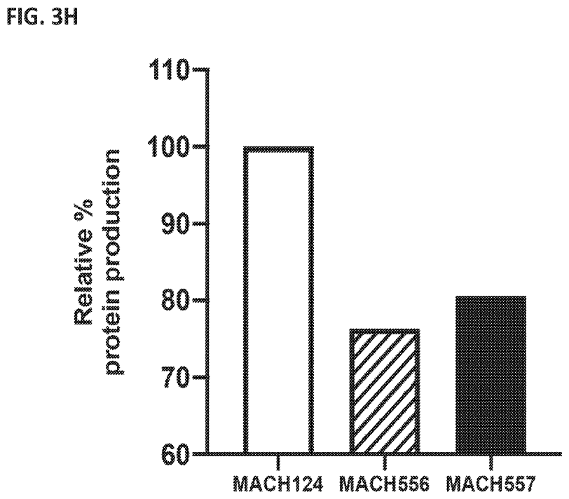

The ability of ADAS to express GFP in an inducer-dependent manner was assessed as described in Example 4C. FIGS. 3A and 3B show an inducer-dependent increase in the transcription and translation of GFP in MACH124, MACH556, and MACH557 ADAS that persisted over 12 hours. In the absence of the inducer (uninduced) the ADAS did not produce GFP. Data in FIGS. 3A and 3B are normalized as described in Example 4C. While ADAS derived from all three min locus deletion strains were able to express GFP upon induction, ADAS derived from MACH124 (BW25113 AminCDE) produced a higher GFP signal over time. At 12 hours, the MACH124 ADAS GFP signal was ˜119% higher than that of MACH557 ADAS and ˜186% higher than that of MACH556 ADAS. ADAS generated from AminCDE parents thus express higher levels of GFP upon induction than those generated from AminC, or AminD parents.

The raw GFP production curves observed for MACH124, MACH556, and MACH557 were used to compare the total protein production and the average rate of protein production for ADAS purified from each strain and incubated in the presence of inducer. A regression line was fit to each data set, and the area under the curve, which reflects total GFP production over 12 hours, was calculated using GraphPad Prism. MACH124 ADAS produced significantly more GFP than MACH556 or MACH557 ADAS (FIG. 3D). To assess the average rate of GFP production over the course of the experiment, the area under each curve was divided by the duration of the experiment. The average rate of GFP production over 12 hours was significantly greater for MACH124 ADAS than MACH556 or MACH557 ADAS (FIG. 3E). Finally, the total amount of GFP produced by MACH556 and MACH 557 ADAS was expressed as a percentage of the GFP produced by MACH124 ADAS, which was normalized to 100%. These data are plotted in (FIG. 3F). MACH124 (AminCDE) ADAS produced a greater total amount of GFP than either of MACH556 (AminC) and MACH557 (AminD).

### C. GFP Expression Assayed Via Immunoblotting

The MACH124, MACH556, and MACH557 ADAS were incubated in 50% LB culture medium with 50 ug/mL Carbenicllin and in the presence or absence of anhydrotetracycline (1 ug/mL) in an Eppendorf ThermoMixer® at 37° C., 800 rpm for 24 hours. Immunoblotting was performed as described in Example 3. After 24 hours, the ADAS were centrifuged at 20,000×g for 10 minutes at 4° C. The supernatant was removed and the pelleted ADAS were resuspended in lysis buffer (1x BugBuster™ Protein Extraction Reagent (MilliporeSigma™) with 1× NuPAGE™ LDS Sample Buffer (Thermo Fisher)) and heated at 85° C. for 2 minutes. An equal volume of lysed ADAS was then loaded into a 4-12% BisTris polyacrylamide gel. Proteins on the gel were resolved, transferred to a nitrocellulose membrane, and incubated in Intercept® PBS Blocking Buffer for 60 minutes at room temperature. The membrane was incubated with primary antibody (Mouse anti-GroEL at a 1:500 dilution and Rabbit anti-GFP at a 1:500 dilution) for 60 minutes at room temperature. Antibodies were resuspended in Intercept® PBS Blocking Buffer supplemented with 0.2% TWEENR 20. The membrane was subsequently washed three times in 1x PBS+0.05% TWEEN® 20 followed by incubation in Intercept PBS blocking buffer+TWEEN® 20 supplemented with relevant secondary antibodies (Goat anti-Mouse 800 and Goat anti-Rabbit 680 both at a 1:5,000 dilution). The membrane was washed three times in 1× PBS+0.05% TWEENR 20 and imaged on an Invitrogen™ iBright™ Imager (Thermo Fisher) (FIG. 3C). The band intensity was quantitated via densitometry (FIG. 3C, center panel) and GFP intensity was expressed normalized against the loading control, GroEL (FIG. 3C, lower panel). MACH124 ADAS demonstrated increased production of GFP compared to MACH556 and MACH557 ADAS, indicating that MACH124 (AminCDE) shows higher activity in terms of protein production.

### Example 6: ADAS with Membrane Displayed Proteins

This example demonstrates the membrane display of nanobodies as a model targeting agent for ADAS. Nanobodies are the smallest known functional antibody fragments, and recent work has shown that they can be expressed on the surface of E. coli cells (Salema and Fernandez, Microb Biotechnol, 10 (6), 2017). Surface nanobodies can efficiently bind to target proteins and can be used to enhance cell-specific binding affinity. This example demonstrates that targeting agents (in this case nanobodies) can be expressed on the surface of ADAS.

A. Production of E. coli Strains Containing a HER2 Targeting Nanobody

To construct ADAS capable of targeting the HER2 receptor (associated with breast cancer), plasmids (pNeae-NB2) (Table 3) were synthesized with nanobody sequences fused to the intimin gene of EHEC O157: H7 strain EDL933stx-. Specifically, 583 amino acids of the N-terminal portion of the intimin gene (Neae) were fused to an E-tag (SEQ ID NO: 5), a glycine-glycine-serine linker, the NB2 nanobody sequence (SEQ ID NO: 6), a serine-glycine linker, and a C-terminal FLAG-tag (SEQ ID NO: 7). This sequence was codon-optimized for expression in E. coli and cloned into a plasmid containing the TetA promoter, the TetR repressor (which represses the TetA promoter), a CloDF13 origin of replication, and a kanamycin resistance gene for antibiotic selection to create pNeae-NB2 (see Table 3). When anhydrotetracycline was added to the culture supernatant, TetR-repression of the TetA promoter was relieved and the Neae-NB2 fusion protein was expressed. The Neae-NB2 fusion protein assembles into the outer membrane of the E. coli parent bacterial cell bearing the plasmid, with the NB2 nanobody displayed outward. The pNeae-NB2 plasmid was transformed into an E. coli BW25113 strain carrying a deletion of the minCDE locus with a chloramphenicol resistance cassette and selected for with addition of 50 μg/mL Kanamycin and 35 μg/mL chloramphenicol in the growth media to create MACH284 (see Table 2).

B. Production of ADAS with HER2 Nanobody Expression

MACH060 (unmodified, negative control ADAS) and MACH284 ADAS with targeting antibodies on their surface were prepared and purified by the process outlined in Example 2, with a slight modification to the growth medium. Here, anhydrotetracycline was added to the culture to a final concentration of 50 ng/ml during growth so that as ADAS were produced from parents they expressed the targeting Neae-NB2 fusion protein from the plasmid.

### C. Confirmation of Display of HER2 Nanobody on ADAS Surface

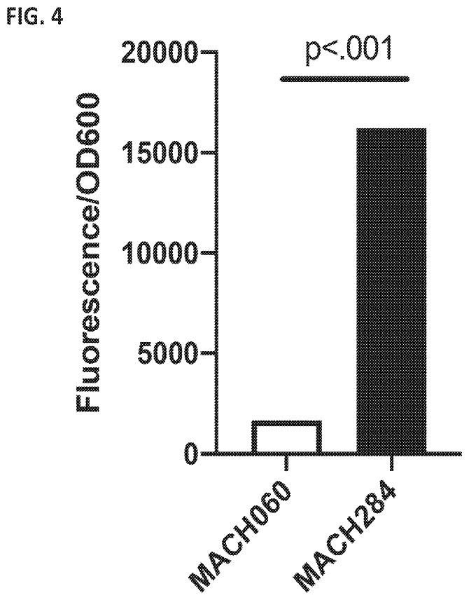

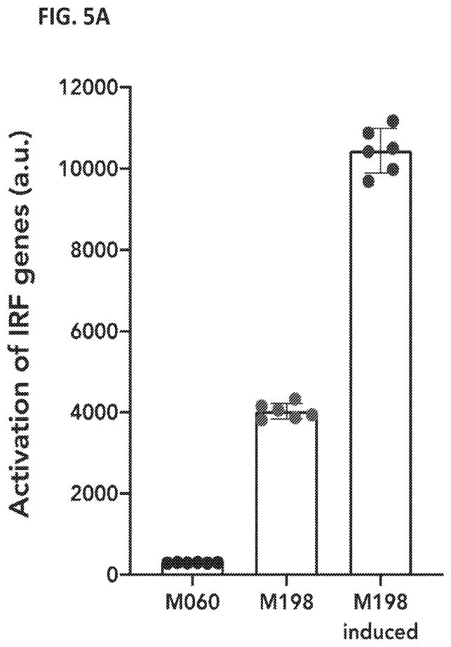

To confirm the expression of Neae-NB2 on the surface of the ADAS, we performed immunofluorescent labeling. ADAS from MACH060 and MACH284 were diluted to a concentration of ˜5×10⁸ particles/mL in PBS as determined by analysis using a Spectradyne® nCS1™ Nanoparticle Analyzer. An antibody targeting the E-tag feature in the Neae-NB2 fusion (Abcam, ab3397) was added to the samples to a final concentration of 5 μg/mL in 1 mL of the samples. These samples were mixed gently and left to incubate for 2.5 hours on ice. After incubation, the ADAS were collected by centrifugation at 15,000×g for 10 minutes at 4° C. Supernatant was discarded and the pellet was gently resuspended in 800 μL of PBS+0.05% v/v TWEEN® 20. This wash was repeated twice and the final pellet was resuspended in 0.5 mL of PBS+0.05% TWEEN® 20. 2.5 UL of Donkey anti-Rabbit conjugated to DyLight® 550 fluorophore (Abcam, ab98489) was added to the sample, mixed gently, and incubated on ice for 1 hour. After this incubation, ADAS were collected by centrifugation at 15,000×g for 10 minutes at 4° C. The supernatant was discarded and the pellet was resuspended in 500 μL of PBS+0.05% TWEENR 20. This wash was repeated three times in total and the final pellet was resuspended in 250 μL of the buffer. 200 μL of sample was removed into a black-walled, clear-bottomed 96 well polypropylene plate. This plate was then read for optical density at 600 nm and DyLight® 550 fluorescence (excitation: 487 nm, emission: 528 nm) using an area scan of the well. FIG. 4 shows the results of the labeling experiment using DyLight® 550 fluorescence normalized to optical density. While the wild-type MACH060 ADAS showed negligible fluorescence, the Neae-NB2 nanobody-displaying MACH284 ADAS showed a statistically significant, nearly 10-fold increase in fluorescence. Thus, the targeting nanobody was effectively expressed and displayed on the outer membrane of ADAS.

### Example 7: Cargo Production and Delivery by ADAS

This example demonstrates that ADAS of the invention are capable of producing and delivering a cargo that has a specific effect on a target cell or organism. As a model we created E. coli ADAS that generate specific cyclic dinucleotides (CDNs). In particular, the ADAS were induced to produce bacterial nucleotides c-di-AMP that engage the Stimulator of Interferon Genes (STING) pathway via the RECON (REductase CONtrolling NF-κB) of mammalian cells (Witte et al., Mol Cell, 30 (2): 167-178, 2008), through expression of a heterologous enzyme. This example further demonstrates that an ADAS can express an enzyme that catalyzes the production of a cargo, which cargo can then be delivered to a target cell.

### A. Construction of STING Agonist E. Coli ADAS

To create ADAS capable of activating the STING pathway, a diadenylate cyclase was expressed in a BW25113 E. coli AminCDE:: CamR (MACH060) strain, as prepared in Example 1. To do so, the amino acid sequence of the diadenylate cyclase A (DacA) protein of Listeria monocytogenes (Accession number: Q8Y5E4) was codon-optimized for expression in E. coli, de novo synthesized by Integrated DNA technologies, and cloned into a plasmid containing the TetA promoter (pTet), the TetR repressor (which represses the TetA promoter), a pMB1 origin of replication, and a beta-lactamase gene for antibiotic selection, yielding the pSTING plasmid (Table 3). This plasmid was transformed into MACH060 (BW25113 E. coli AminCDE:: CamR) and selected for using 100 μg/mL Carbenicillin and 35 μg/mL Chloramphenicol in the growth media to yield the strain MACH198 (Table 2). When induced with anhydrotetracycline added to the growth media, MACH198 produced DacA, which catalyzes the condensation of two ATP molecules into the potent STING activator cyclic-di-AMP.

### B. Purification of MACH060 and MACH198 ADAS

For STING activation assays, MACH060 and MACH198 were prepared using the methods outlined in Example 2 with minor modifications. Briefly, two cultures of MACH198 were used: one grown normally (uninduced), and one with 200 ng/ml anhydrotetracycline added (induced). The ADAS were concentrated and harvested using an 0.2 μm bottle top filter and resuspended in THP1-Dual™ growth media (InvivoGen) to a final concentration of approximately 109 particles/mL. The residual parent burden, as assessed by the CFU plating method outlined in Example 2, showed less than 200 CFU/mL of parent bacterial cells.

### C. Assessment of Function Using Mammalian Cell Assays

The THP1-Dual™ monocyte cell line (Invivogen) was used to assess activation of the STING pathway using a luminescence readout. THP1-Dual™ cells feature the Lucia gene, a secreted luciferase reporter gene, under the control of an ISG54 minimal promoter in conjunction with five IFN-stimulated response elements. When the IFN-stimulated response elements are activated, e.g., by exposure to c-di-AMP (e.g., by phagocytosis of ADAS comprising c-di-AMP (e.g., MACH198 ADAS)), the ISG54 minimal promoter is activated and luciferase is transcribed, translated, and secreted of luciferase into the cell media. To quantify luciferase, the substrate QUANTI-Luc™ (InvivoGen) is added to the cell media. QUANTI-Luc™ contains the luciferase substrate coelenterazine, which produces a light signal when hydrolyzed by luciferase that may be quantified using a plate reader.

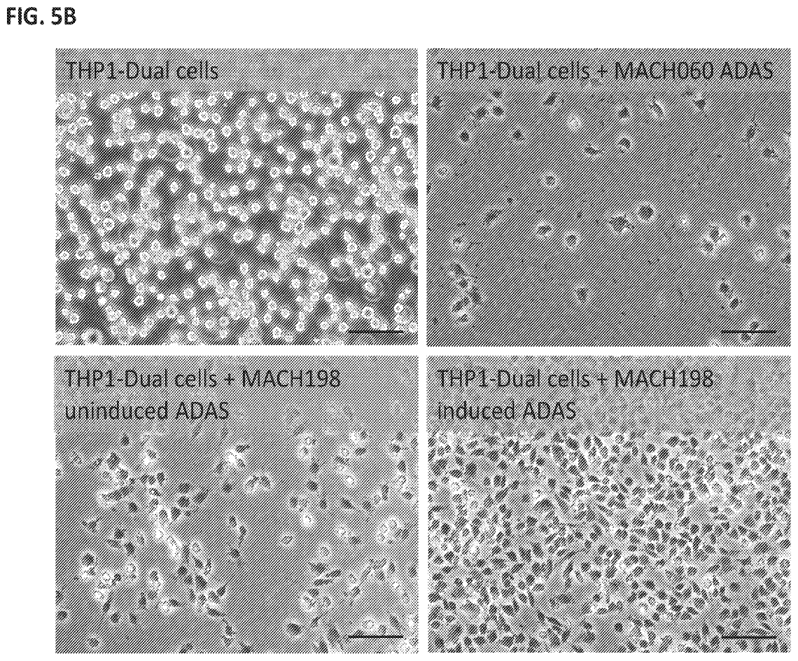

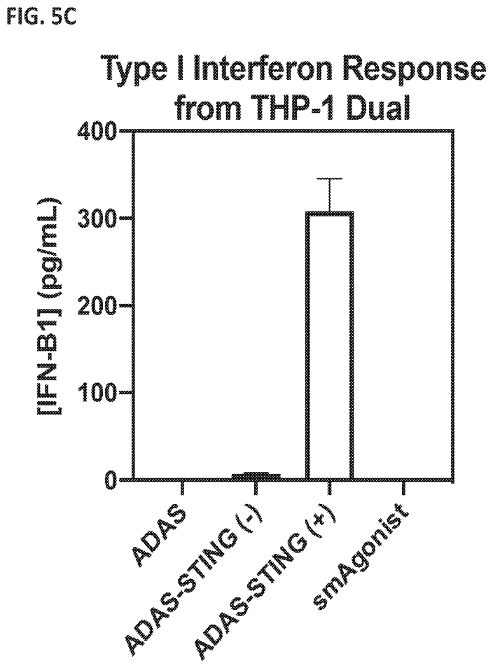

To assess whether ADAS activate the STING pathway in THP1-Dual™ cells, THP1-Dual™ cells were cultured per InvivoGen's specifications and were seeded in a 96 well plate at a concentration of 5×10⁵ cells/mL at 125 μL per well. ADAS from MACH060, uninduced MACH198, and induced MACH198 (Table 2) were added ˜4×10⁷ ADAS/mL at a volume of 100 μL per well. After incubating at 37° C. for 42 hours, the plate was centrifuged at 300×g for 5 minutes. Finally, 20 μL of media was removed from each well and placed in a white-walled plate containing 50 μL per well of QUANTI-Luc™. Luminescence was read with a plate reader immediately. FIG. 5A shows the average luminescence of samples from each experimental condition normalized to the volume of ADAS added, as determined using a Spectradyne® nCS1IM Nanoparticle Analyzer as described in Example 1. Each ADAS condition included 6 biological replicates. Little to no luminescence signal was obtained from the MACH060 ADAS, which did not contain the DacA gene and largely did not trigger IFN-stimulated response elements in the THP1-Dual™ cells. However, MACH198 ADAS, which contained the DacA gene under the control of pTet, did stimulate the THP1-Dual cells. The highest stimulation was observed in the samples in which DacA expression was induced by culturing with anhydrotetracycline. Furthermore, when monocytes experience stimulation of IFN genes the cells assume an activated phenotype and transition from non-adherent monocytes in suspension to activated, adherent macrophages. We observed this transition in the samples exposed to various ADAS populations. FIG. 5B shows that the activated adherent phenotype is most evident in the samples in which THP1-Dual|M cells were treated with anhydrotetracyline-induced MACH198 ADAS. Elicitation of the downstream Type I interferon response is essential to the function of the STING pathway. The signature of this response is the secretion of Type I interferons, such as interferon beta (IFNB1). To further elucidate the ability of ADAS to induce a functional STING response, IFNB1 was quantified for each sample using a Human IFN-beta Quantikine ELISA Kit (DIFNBO, R&D Systems®). Cell supernatants were aspirated from the assayed wells above and diluted down into the linear range of the ELISA. The ELISA was performed according to the manufacturer instructions. FIG. 5C shows the results of the ELISA. While MACH060 ADAS and the small molecule STING agonist (provided as a control) showed IFNB1 levels below the limit of detection of the ELISA assay (<5 μg/mL), the uninduced MACH198 ADAS showed trace amounts of IFNB1 (˜7 ng/ml) and the induced MACH198 showed a more than 40× increase in IFNB1 secreted into the cell supernatant (˜300 ng/ml). The ADAS thus demonstrated the ability to make a cargo through expression of a heterologous enzyme, which cargo was delivered to a target call and induced a functional (in this case immunomodulatory) response.

### Example 8: Loading of E. Coli ADAS with RNA Cargo

This example describes the expression and packaging of RNA cargo, in this case dsRNA targeting the gene for chymotrypsin (chyl) from Plutella xylostella into ADAS.

A. Construction of E. coli Parent Lines Containing a dsRNA

To construct ADAS packaged with dsRNA, a 412 bp sequence (SEQ ID NO: 8) corresponding to a portion of the coding region of the Plutella xylostella chyl gene for chymotrypsin was cloned into the plasmid L4440 (Fire et al., Nature, 391 (6669): 806-811, 1999), containing dual T7 promoters, a pMB1 origin of replication, and an ampicillin resistance gene for antibiotic selection yielding pRNAi (see Table 3). This plasmid was transformed into MACH300 (Table 2), an E. coli HT115 (DE3) AminCDE strain containing a chloramphenicol resistance cassette, and selected for with the addition of 50 μg/mL Carbenicillin and 35 μg/mL Chloramphenicol in the growth media to create MACH301 (Table 2). MACH301 also carries a disruption in the mnc gene, encoding RNase III, which degrades dsRNA, with a tetracycline resistance cassette. This strain additionally carries the DE3 prophage, which encodes a copy of the T7 RNA polymerase gene behind the lac promoter. When isopropyl β-d-1-thiogalactopyranoside (IPTG) is added to the media, the T7 RNA polymerase is expressed. The T7 RNA polymerase then transcribes the inserted chyl sequence from both T7 promoters, creating a corresponding dsRNA.

B. Production of ADAS with dsRNA

MACH301 ADAS loaded with dsRNA were prepared and purified by the process outlined in Example 2, with modifications to the growth conditions. Briefly, overnight cultures of MACH301 were diluted 1:200 in LB with 50 μg/mL Carbenicillin and grown to OD600=0.4. IPTG was subsequently added to the culture to a final concentration of 1 mM and growth continued for 4 hours so that as ADAS were produced from parents they expressed the dsRNA construct from the plasmid.

C. Quantification of dsRNA Loading in ADAS

To quantify the levels of dsRNA in ADAS, we analyzed the nucleic acid content by gel electrophoresis. ADAS at a concentration of about 3.3×10¹⁰ particles/mL in PBS, as determined by Spectradyne® nCS1™ Nanoparticle Analyzer analysis as described in Example 3, were lysed via heat treatment at 80° C. for 20 minutes. This treatment led to release of the nucleic acids and easy visualization via agarose gel electrophoresis. 20 μL of heat-treated ADAS lysate were run alongside a standard curve created by dilution series of an in vitro transcribed dsRNA at 125 ng/μL on a 2% agarose E-GelIM EX (Thermo Fisher) following the manufacturer's recommended settings. The gel was imaged using a Invitrogen™ iBright™ Imager (Thermo Fisher) on the nucleic acid gel setting. Band intensities were quantified with ImageJ. The amount of loaded dsRNA was calculated to be 97 ng per 109 ADAS. This example demonstrates that ADAS can effectively be loaded with RNA cargo.

### Example 9: Delivery of E. Coli ADAS to Lepidoptera

This example describes fluorescent labeling and visualization of ADAS within a lepidopteran insect, specifically the European Corn Borer, after addition of ADAS to an artificial diet.

### A. Fluorescent Labelling of ADAS for Visualization

To visualize ADAS in the European Corn Borer, we fluorescently labeled ADAS with an NHS ester fluorescent dye. MACH301 ADAS were prepared and purified by the process outlined in Example 8.

30 μL of 0.5 M Sodium Borate was added to 1 mL of ADAS at a concentration of 1×10¹¹ as measured by Spectradyne® nCS1™ Nanoparticle Analyzer analysis. DyLight™ 800 NHS Ester (ThermoFisher), which reacted with amine-containing molecules on the outer membrane surface of the ADAS (e.g. primary amines of proteins), was dissolved in anhydrous dimethylformamide at a concentration of 10 mg/ml. 20 μL of DyLight™ 800 NHS Ester stock was added to the ADAS in a 1.5 mL tube and briefly vortexed. The subsequent reaction mixture was wrapped in foil and placed on a rocker for one hour at room temperature. To remove excess dye, ADAS were pelleted at 20,000×g for 10 minutes at room temperature and resuspended in 1 mL of PBS. This wash was repeated 3 times, and ADAS were resuspended in a final volume of 1 mL of PBS.

### B. Feeding Labelled ADAS to European Corn Borer (ECB)

European Corn Borer (Ostrinia nubilalis) eggs were obtained from Benzon Research Inc. and were reared on an artificial diet (general noctuid diet) purchased from Benzon Research Inc. The diet was prepared as follows: 162 g of the general noctuid diet powder was added to boiling water; the contents were mixed thoroughly for 15 minutes while keeping the temperature between 80° C. and 90° C.; the mixture was cooled down to 70° C., and 5 mL of linseed oil was added and mixed in thoroughly; and the food was dispensed into rearing containers and allowed to cool and solidify.

The ECB eggs were placed on the diet and allowed to hatch and feed. All rearing containers were maintained at 25° C., 16 hour: 8 hour light: dark cycle, and 50-60% humidity. Once the larvae reached the 2nd instar stage, they were used for the feeding assay. To prepare the setup for feeding, 25 mL of the general noctuid diet was prepared and poured (while hot, 70° C.) into the lid of a 0.4 liter container (Sistema 1543 Klip It box), and immediately placed a 48 well PCR plate with the bottoms cut off (removed about 2 mm from the bottom) into the food while solidifying: this created individual wells with the right amount of diet for this feeding assay for 2nd instar larvae. To administer the DyLight™ 800 NHS Ester-labeled ADAS or PBS as a control, 2 μL of the labeled ADAS or PBS was added to 5 wells separately and allowed to dry. Individual 2nd instar larvae were then added to each well and allowed to feed for 1 hour.

### C. Visualization of Fluorescent ADAS in European Corn Borer

To visualize ADAS in ECB larvae, the larvae fed with PBS or DyLight™ 800 NHS Ester-labeled ADAS were removed from the well, placed on a sticky tape to immobilize them, and then placed on the imaging surface. Both the 700 nm and 800 nm channels were used for scanning the larvae. The larvae autofluoresce in the 700 nm channel; hence, this channel was used to locate the larvae on the imager. The 800 nm channel was used to detect labeled ADAS in the larvae.

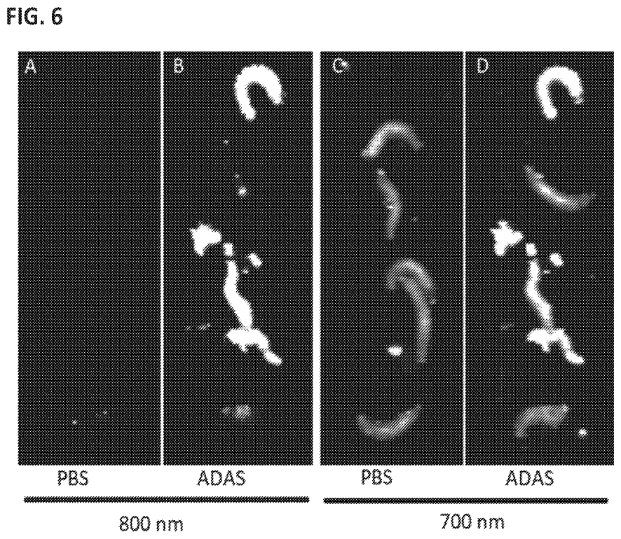

FIG. 6 shows the results of the feeding experiment, with very little signal in the 800 nm channel in the PBS fed larvae, and a strong fluorescent signal present in larvae fed DyLight™ 800 NHS Ester-labeled MACH301 ADAS. Thus, ADAS are eaten by and present within the European Corn Borer when added to diet.

### Example 10: Storage of ADAS

In this example, we demonstrated methods to formulate and store ADAS using E. coli as a model ADAs-producing bacterium. E. coli ADAS with inducible GFP plasmids were synthesized and purified according to the methods provided in Examples 1-3. They were then stored in various conditions to evaluate for their ability to be reconstituted and survive.

### A. Longitudinal Storage of ADAS in Cold Conditions

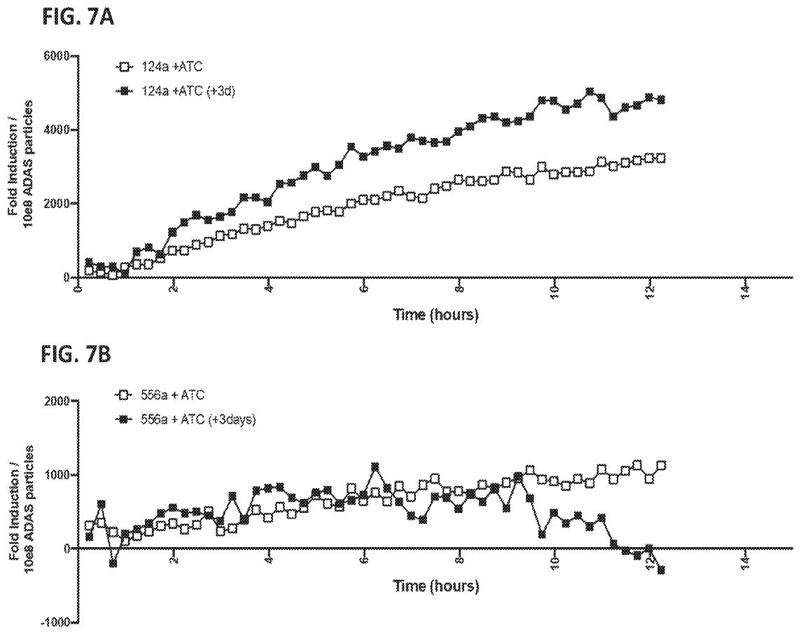

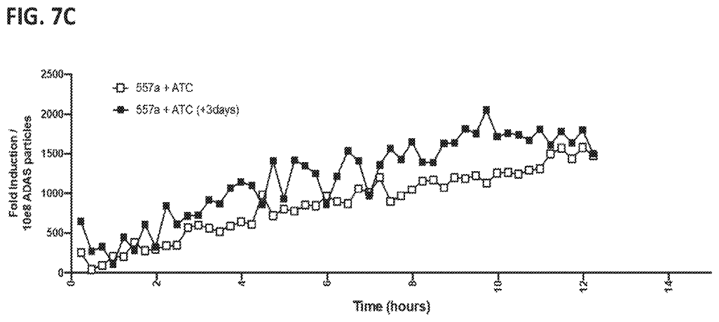

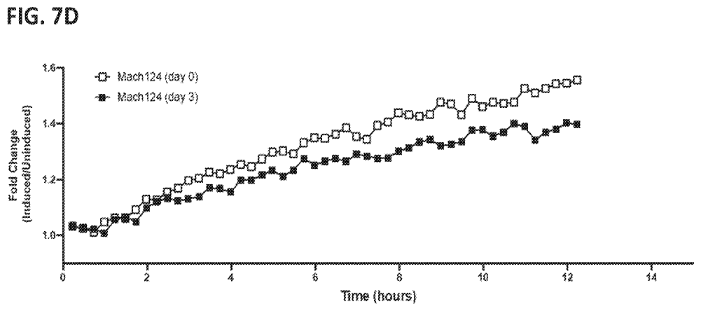

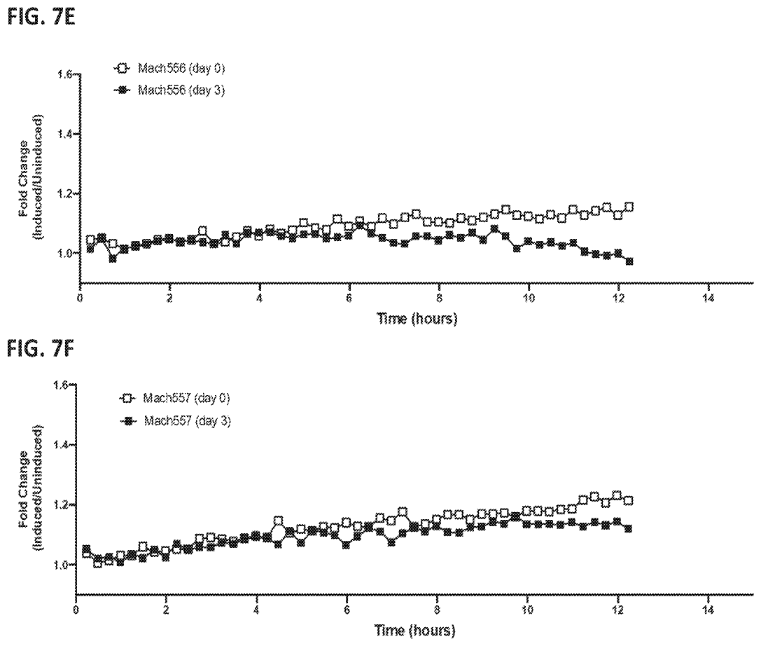

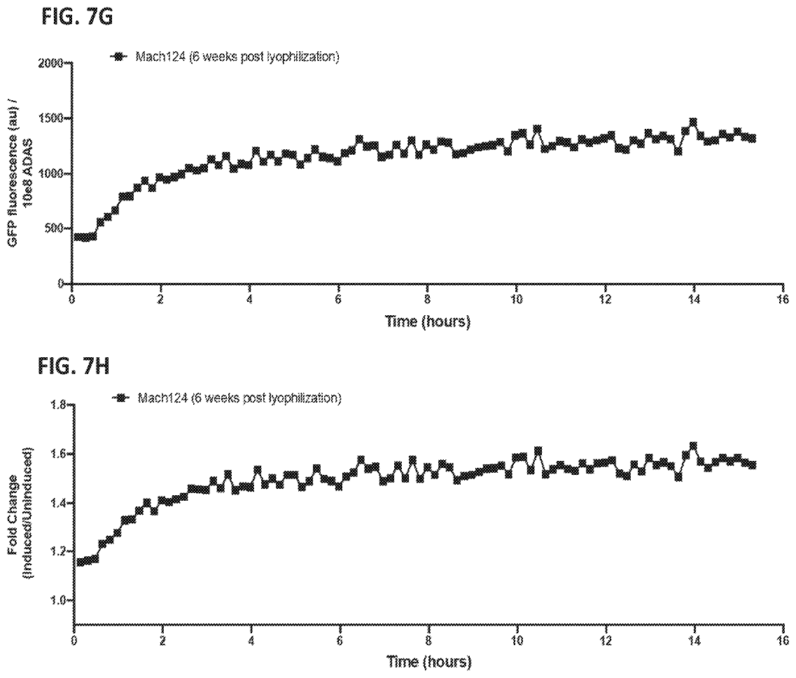

In this example, the ability of ADAS to express GFP in an inducible manner after storage at 4° C. for 0 and 3 days was assessed. ADAS were derived from three strains that contain the pGFP plasmid (Table 3) driving GFP expression under the control of a tetracycline inducible promoter: MACH124 (BW25113 AminCDE), MACH556 (BW25113 AminC), and MACH557 (BW25113 AminD): see Table 2 for genotype information. ADAS were purified as indicated in Example 2, using selective outgrowth from a concentrated culture supernatant. For the purified MACH124 preparation, the concentration of ADAS was determined to be 2.4×10⁹ ADAS/mL immediately after purification and 2.18×10⁹ ADAS/mL after 3 days in storage. For the purified MACH556 preparation, the concentration of ADAS was determined to be 1.86×10⁹ ADAS/mL immediately after purification and 2.2×10⁹ ADAS/mL after 3 days in storage. For the purified MACH557 preparation, the concentration of ADAS was determined to be 1.98×10⁹ ADAS/mL immediately after purification and 2.08×10⁹ ADAS/mL after 3 days in storage. All ADAS concentration measurements were taken using the Spectradyne® nCS1™ Nanoparticle Analyzer (outlined in Example 3). The purified ADAS preparations were subjected to the nanoparticle characterization and viable cell plating procedures described in Example 3. For each preparation, the viable cell burden was determined to be at or below the limit of detection (100 CFU/mL) by CFU plating, as described in Example 3. The ability of stored ADAS to express GFP in an inducer-dependent manner was assessed as described in Example 4C. At day 0 (no days in storage), an inducer-dependent GFP signal was observed that increased over 12 hours in all three strains. After 3 days in storage, we observed an inducer-dependent increase in the transcription and translation of GFP in MACH124 and MACH557 ADAS that persisted over 12 hours (FIGS. 7A-7F). MACH556 showed an inducer-dependent increase in GFP signal until ˜6 hours, and then the signal declined back to uninduced levels by 12 hours. In the absence of the inducer (uninduced), there was no increase in the GFP signal for any tested strain. Data in FIGS. 7A-7F are normalized as described in Example 4C.

### B. Lyophilization and Reconstitution of ADAS

To examine the capacity of ADAS to perform work after lyophilization, ATP levels were measured in rehydrated ADAS 1, 2 or 6 weeks post-lyophilization. Further, the ability of lyophilized ADAS to transcribe and translate the target gene GFP after rehydration was also assessed. ADAS from MACH124 were purified as indicated in Example 2. To lyophilize ADAS, purified ADAS were pelleted at 21,000×g for 20 minutes and resuspended in Microbial Lyophilization Buffer (OPS Diagnostics). The ADAS were flash-frozen in liquid nitrogen and freeze-dried for 18 hours on the auto setting of a Labconco™ FreeZone Freeze Dryer set to a vacuum of 0.3 mbarr. Freeze-dried ADAS were stored in the dark in a Ziploc bag at room temperature until rehydration. To determine ATP levels of ADAS post-lyophilization, ADAS were rehydrated 1, 2, and 6 weeks post-lyophilization, and ATP levels were measured using the BacTiter-Glo™ Microbial Cell Viability Assay Kit (Promega), following the manufacturers' instructions. The assay showed that lyophilized and rehydrated ADAS preserve ATP at similar levels after 1, 2, or 6 weeks of storage, with ATP levels at 2 and 6 weeks increasing slightly (by 1.49 and 1.48 fold, respectively) over ATP levels measured at 1 week. This data demonstrates that ATP levels are maintained by the lyophilization, storage, or rehydration processes.

Additionally, the ability of rehydrated ADAS to transcribe and translate GFP in an inducer-dependent manner was tested using the protocol outlined in Example 4C. FIGS. 7G and 7H show an inducer-dependent increase in the GFP signal in rehydrated MACH124 ADAS that persisted over 15 hours. In the absence of the inducer (uninduced), there was no increase in the GFP signal. Data in FIGS. 7G and 7H are normalized as described in Example 4C.

The activity observed in rehydrated ADAS indicates that ADAS integrity is maintained by the lyophilization process.

### Example 11. Supplemental Methods for Characterization of ADAS

This example describes various supplemental analytical methods that are used to characterize the composition of ADAS and parent cell.

### A. Electron Microscopy

ADAS are purified from parental bacteria, cell debris, and endotoxins as described in Example 12. To visual periplasmic structures including flagella and secretion systems, the ADAS are osmotically-shocked in similar manner to previous methods (H C Neu and L A Heppel. 240:3685-3692, 1965) after separation. The separated periplasmic structures are then visualized in the transmission electron microscope (JEOL, Tokyo) following the protocol from Wu et al., Analyst, 2015.

### B. Fluorescence and Optical Microscopy

ADAS and parental bacteria are visualized using a fluorescence equipment upright microscope (Leica, Zeiss, CCD camera, and broad-spectrum light source). Samples are imaged live within tissue culture multi-wells of 6-, 12-, 24-, or 96-well format (Thermo Fisher), transwell inserts (Corning), or agar-coated polystyrene petri dishes (Thermo Fisher). Additionally, samples can be fixed in these containers or on glass coverslips for further analysis.

### C. OD600 Measurement of Concentration

To quantify the number of ADAS or parent bacteria present in a given volume of medium, optical density is measured at OD600 and the following equation is employed:

Number of ADAS/ml=OD600×A×B

where A is the relative size of ADAS compared to parental cells and B is the calibration curve factor relating to OD600 of ADAS versus their LPS content. This is derived from the standard AD600 measurement for bacterial cells.

### D. Nanoparticle Tracking Analysis of ADAS and Parent Bacteria

Nanoparticle tracking analysis is typically used to measure purity of vesicles and is modified for measuring ADAS purity. Briefly, nanoparticle tracking is done using the NanoSight LM10 system (NanoSight Ltd, Amesbury, UK) configured with a laser and a high sensitivity digital camera. Videos are collected and analyzed using standard software, using expected particle size as input. Each sample is diluted to a known concentration in the range of 108 particles/ml (using spectrophotometric quantification) and administered and recorded under controlled flow using the pump system in NanoSight. The camera is operated at maximum frame rate and resolution. The number of particles in the correct size range are quantified, along with a percentage of total particles (e.g. parent cells) outside the size range.

### E. Dynamic Light Scattering and Zeta Potential

ADAS and parental bacteria population size distributions are measured using dynamic light scattering (DLS) (Zetasizer Nano S from Malvern Instruments Ltd.). By exposing ADAS suspensions to light, the hydrodynamic radius is measured by relating the scattered light intensity to the diffusion coefficient of the object in solution using published protocols (Jorge Stetefield, Biophys Rev (2016) 8:409-427). These studies have been previously performed with nanoparticles, bacteria, proteins, and nucleic acids in a similar fashion. Disposable cuvettes are typically used for ADAS size measurements to maintain sterility and avoid cross-sample contamination. For some applications that require organic solvents, reusable glass cuvettes are used with careful cleaning protocols that include sequential cleanings of detergent (Alconox), 5% Acetic Acid, DI water, and 70% ethanol followed by a thorough drying process.

### F. Immunofluorescence Staining:

ADAS are isolated from parent bacterial using methods described in Example 12. ADAS are diluted and spun at 500 g onto clean glass coverslips and fixed in 4% paraformaldehyde in PBS solution for 20 minutes. Samples are washed, incubated in staining solution of antibodies according to the manufacturer's instructions, mounted onto a slide with antifade mountant following manufacturer's instructions, dried, and imaged under a confocal microscope. ImageJ is used to process the images.

### G. Flow Cytometry:

Parent bacteria and ADAS are analyzed using flow cytometry on a NanoFCM (NanoFCM, China) following the manufacturer's instructions. Using fluorescence ADAS, the NanoFCM is used in either one- or two-color mode (exposure at 488 nm and 555 nm wavelengths) to confirm various properties of the ADAS, including plasmid uptake, protein expression, and purity. For example, a fluorescent lipophilic dye, such as DiOC6 (ICN Biomedical) is incorporated into ADAS membranes, as described by the manufacturer. ADAS are sorted and purified as explained in Example 12 and then washed in cold phosphate-buffered saline (pH=7), repelleted (40,000 g, 5 min, 4° C.), and diluted to (1E5, 1E6, 1E7) ADAS/mL and DiOC6 fluorescence intensity is measured at 488 nm excitation and 535 emission.

### H. PCR

Purified ADAS are lysed and DNA is purified using a QIAquick PCR purification kit according to manufacturer's directions (Qiagen). Oligonucleotide primers 23S-sense (59 GAA AGG CGC GCG ATA CAG 39) and 23S-antisense (59 GTC CCG CCC TAC TCA TCG A 39) are used to amplify a 70-bp fragment of the 23S ribosomal RNA gene, present in seven copies in the E. coli genome as described by Vilalta et al., Anal Biochem, 2001. Amplification reactions are carried out using TaqMan reagents (ThermoFisher Scientific) according to manufacturer's instructions.

### I. RNA SEQ

Whole transcriptome analysis is done using RNAseq as described by Giannoukos et al., Genome Biol, 2012. Briefly, ADAS are purified to an OD600 of ˜0.5 in LB broth and harvested by centrifugation at 4,000×g for 10 minutes at room temperature. Pellets are resuspended in 25 ml of RNAlater (Ambion, Carlsbad, CA, USA). The tubes are agitated on a rotator at 4° C. overnight, centrifuged at 4,000×g for 10 minutes, placed in an ethanol/dry ice bath to flash freeze the pellet and stored at −80° C.

RNA extraction is done using Ion Total RNA-seq Kit v2 (ThermoFisher Scientific) according to manufacturer's instructions. Enzymatic reactions using the mRNA-ONLY Prokaryotic mRNA Isolation Kit (Epicentre) are performed according to the manufacturer's specifications. The Ovation Prokaryotic RNA-Seq System (NuGEN Technologies, Inc., San Carlos, CA, USA) is used as follows. Intact RNA is DNase treated as described above and synthesized into cDNA according to the manufacturer's protocol.

The purified products are size selected on a gel (approximately 300 to 450 bp). Samples are enriched with Illumina PE1.0 and PE2.0 primers (1 UM each), 1x of AccuPrime PCR buffer | (10×), 0.5 U of AccuPrime Taq High Fidelity polymerase (5 U/μL; Invitrogen) in a final volume of 25 μL. Enriched reactions are purified using Agencourt AMPure XP beads (0.8× the reaction volume). Libraries are sequenced on either Illumina GAll or Hi-Seq instruments. The raw reads of RNA-seq data are processed using the Picard pipeline. Briefly, the reads are aligned and assigned to the reference genomes using the program HISAT2 Sequence data for E. coli are aligned to the respective genome sequences. HISAT2-aligned reads are then analyzed and assigned to individual genes according to the genome annotations provided by GenBank.

### J. DNA SEQ

Residual DNA from parent lines is measured using the resDNASEQ Quantitative E. coli DNA kit (ThermoFisher Scientific) using KingFisher Flex Express 96-deep-well automation platforms to automate the extraction of host-cell line residual DNA according to manufacturer's instructions. Briefly, two wash and one elution plates are prepared, and samples are loaded. The samples are then lysed and processed on KingFisher Flex using the PrepSEQ_resDNA_v1 script. Standard curves are generated using E. coli parent lines, and samples are amplified using a master mix and results are read and analyzed using SDS software.

### K. Gels

DNA from ADAS and E. coli parent lines is extracted using standard protocols and loaded onto agarose gels for size analysis following manufacturer's instructions. Genomic DNA is ˜4.5 Mb, while plasmid DNA is ˜3-5 kb.

### I. ELISA

FluoroSELECT E. coli assay kit (Sigma-Aldrich) is used for ELISA detection of parent cells. The detection system utilizes a fluorogenic substrate which, when hydrolyzed by a specific enzyme (during peptide hydrolysis), produces a fluorescent signal. Briefly, samples are prepared according to manufacturer's instructions, and read using a fluorimeter. After calibration, if measured P1>30,000, sample is positive to parent line cells. If P1<30,000, measure P2. If the numerical value (P2-P1)< (3%×P1), the sample is negative.

### Example 12. Supplemental Methods for Production of E. Coli ADAS

This example describes supplemental methods for the production and characterization of ADAS from Escherichia coli.

A. Production of E. coli ADAS

To create ADAS, E. coli are transfected with a plasmid that overexpress ftsZ protein under the T7 promoter. Alternatively, E. coli mutants are created with disrupted MIN genes by transfection of E. Coli with integrating plasmids. Plasmids are synthesized commercially by Thermo Fisher. Transfection is carried out using standard bacteria transfection processes (Thermo Fisher Molecular Biology Handbook). In short, competent cells are plated on room temperature agar plates. 0.5-2 ng/ml of DNA are added to the competent cells within a vial and incubated for 20-30 minutes. Each tube is then heat shocked by placing the tube in 42° C. water bath for 30-60 seconds to create transient pores in the cell surface. Cells are then plated on agar gels that have been preloaded with selective antibiotic and grown overnight so only bacteria that have been transfected with the plasmid survive. A single colony is picked and cultured at 37° C. with continuous shaking at 120 rpm in LB medium containing 50-100 μg/mL of ampicillin. Over time, the selected colonies proliferate and produce ADAS continuously.

### Example 13. Supplemental Methods for the Purification of ADAS Derived from E. Coli and Efficiency Measurement

This example describes supplemental methods to purify Escherichia coli ADAS from a crude preparation as well as methods to characterize the amount of contaminating live bacteria.

A. Purification of E. coli ADAS

To separate ADAS from the parent bacteria for high purity production, a combination of washing, centrifugation, sterile filtration, and antibiotic treatment is used. Utilizing protocols adopted from previously published methods (Reeve, J 1979, Jivrajani 2013, Rampley et al 2017) with several modifications. Briefly, to purify the solution, a mixture of parent bacteria and ADAS solution is collected and centrifuged at 4° C. (Beckman Coulter) at increasing speeds in 1000 g increments going from 1000 g to 4000 g for 10 minutes at each step in which an increasing fraction of parent cells from the suspension is removed. To further ensure that parent bacteria have been removed, 100 μg/mL of potent antibiotic (ceftriaxone) is added and the sample is incubated with final supernatant solution at 4° C. overnight. The next day, the solution is centrifuged at 400 g at 4° C. to pellet any cellular debris, supernatant rich in ADAS is collected, and then centrifuged at 4000 g for 5 minutes at 4° C. Finally, the solution is sterile filtered using a 0.2 μm membrane filter and ADAS are then resuspended in sterile PBS with Ca2+ and Mg2+ at the desired concentration.

### B. Measuring ADAS Purification Efficiency

At each step of ADAS production, supernatant or sediment is collected and plated on agar plates and incubated at 37° C. to visually confirm that parent cells are being removed from solution. In parallel, using a hemocytometer, the number of ADAS and parent bacteria are counted using optical microscopy. Additionally, to ensure that ADAS do not contain residual DNA, a fluorescence-based assay using NucBlue Live ReadyProbes Reagent (Invitrogen, R37605), which will become UV fluorescent if the ADAS are positive for residual DNA, is performed. The reagent is added as directed by the manufacturer (2 drops per mL of sample) and incubated for 15-30 minutes and washed with PBS solution prior to imaging to remove excess reagent. Upon exposure to 405 nm wavelength light the dye will excite in the blue spectra if it has been coupled to any remaining DNA. As a counterstain, red-fluorescent FM 4-64 dyes (Invitrogen) are used as an intercalating lipophilic membrane-based dye that stains the outer leaflet of the ADAS and parent bacteria. The excitation and emission spectra are separate from the Nuclear stains to ensure a unique signal can be collected. Purity of the sample is calculated as a percentage of ADAS in remaining population=100× (#red- # of blue)/(#red). Additionally, using the dynamic light scattering (DLS) method (Zeta Sizer Malvern), the purity is evaluated by comparing the magnitude of peaks associated with ˜1 μm (parent cells) and ˜500 nm (ADAS) and showing that the parent cell peak approaching zero in samples of increasing purity.

Alternatively, residual ADAS DNA is determined using Quant-iT™ PicoGreen™ DNA Assay Kit (Invitrogen, Cat #P11496) using protocols established by the vendor. ADAS are collected in PBS and lysed using lysis buffer. PicoGreen reagents are mixed and the reaction is observed in the samples using fluorescence measurements.

Alternatively, ADAS are concentrated and spread to 2.5×10¹¹ ADAS/mL in suitable culture media, and 4 mL is plated on a 60 mm plate with suitable growth agar and cultured in suitable conditions. Colonies are counted after 2 days to determine the number of live bacteria per 1012 ADAS.

Alternatively, nanoparticle tracking, Zetasizer, and other size distribution tracking methods are employed to determine the size distribution and check for the presence of a peak of large particles indicating live bacteria following the protocols in Example 11. Alternatively, samples are tested on a qNano Gold (Izon Science Ltd.), which uses tunable resistive pulse sensing to measure particle size, concentration, and charge as they pass through a nanopore.

### Example 14. Preparation of Large E. Coli ADAS

ADAS in Example 12 are prepared through disruption of septation genes leading to asymmetric cell division and production of ADAS from a parental bacterial cell strain. This example demonstrates the production of ADAS using exonucleases that selectively degrade the genome of the parent bacteria directly, which offers several advantages over ADAS preparation described in Example 12, e.g. improved control cytoplasmic composition, increased copy number cargo, and increased ATP.

A. Production of large E. coli ADAS

Two plasmids are constructed, (1) containing an arabinose promotor to overexpress the sbcB or sbcCD genes, which encodes exonucleases that degrade DNAs in many conformations and allow natively expressed RecBCD to digest the genome, and (2) a plasmid containing a cassette that knocks out the recA gene under the control of an IPTG-induced lac promoter. The plasmids are commercially synthesized and (1), (2) or both are transfected into E. coli cells following standard protocols such as the one in Example 12.

### B. Purification of Large E. Coli ADAS

After bacterial culture reaches 600 O.D, the Exo1 gene is activated using 0.1% arabinose and glucose free media or IPTG or both. After 15 mins, 1 hour, 2 hours, and 6 hours the cells are collected and centrifuged at 4° C. for 5 minutes and resuspended in PBS. As described in Example 15, auxotrophic ADAS are used to increase solution purity which is determined using the measures in Examples 2 or 12.

### Example 15. Supplemental Methods for Producing Auxotrophic ADAS-Producing Bacterial Strains to Increase Purity

This example describes the synthesis of ADAS from parent strains that are auxotrophic as a mechanism for reducing the number of viable bacteria contaminating ADAS preparations.

a. Preparation of Auxotrophic E. Coli Parental Bacteria

ADAS with a decreased number of parental bacteria contaminants are created using auxotrophic strains of E. coli, such as arginine synthesis knockout (argA) strain JW2786-1. E. coli ADAS are produced according to the methods of Example 12 but with arginine added to the media. The ADAS are purified using the methods in Example 13, and then stored in arginine-free media.

B. Preparation of Large E. Coli ADAS from Auxotrophic Strains

Large ADAS from auxotrophic parent strains are created using an auxotrophic strain of E. coli, such as arginine synthesis knockout (argA) strain JW2786-1. E. coli large ADAS are produced according to the methods of Example 14 but cultured in arginine containing media. The ADAS are then purified using the methods in Example 14.

### C. Demonstrating Increased Purity of Auxotrophic Large and Regular ADAS

Large and regular ADAS are prepared using the methods above. ADAS from non-auxotrophic parent strains and non-auxotrophic and large ADAS are also prepared using the methods in Example 12, Example 13 and Example 14. ADAS purity is measured using the methods in Example 15.

### Example 16. Supplemental Methods for Measuring ADAS Activity

This example describes supplemental methods for measuring the activity of ADAS.

a. ATP Measurement

A sample of ADAS is split into two. ATP of half the sample is measured via the BacTiter Glo assay (Promega) following the manufacturer's instructions. Size and concentration of the other half is measured either through nanoparticle tracking or NanoFCM, using protocols from Example 11. The total membrane surface area of the ADAS is calculated using the appropriate formulas, and the ATP amount from the BacTiter Glo assay is divided by the area to yield the ATP per unit ADAS surface area.

### B. ATP Drop Measurement

The ADAS are incubated at 37° C. for those that are mammalian-relevant and 30° C. for those that are not, and the ratio of ATP concentration between measurements taken at preparation and after 24 hours is measured using the methods in part a).

### C. Lifetime Index Measurement

ADAS are synthesized with a functional GFP plasmid with species-appropriate promoters. GFP concentration is measured relative to the number of ADAS, average number of plasmids per ADAS, and solution volume with a plate reader at 30 minutes and 24 hours. The lifetime index is calculated as the ratio of GFP production rate at 24 hours to 30 minutes.

### D. ATP Consumption Measurement

To evaluate the activity of the parent bacteria and the ADAS, ATP production rate is measured. ATP production rate is measured using a Seahorse XF (Agilent) following manufacturer's instructions. Alternatively, a sample of ADAS is split in half. Half of the sample is treated with an ATP synthesis inhibitor, such as dicyclohexylcarbodiimide. Both halves are then assayed with BacTiter-Glo (Promega) according to the manufacturer's instructions. The difference in the two samples is considered to be the ATP generation rate.

### Example 17. Supplemental Methods for Storage of ADAS Derived from E. Coli

This example describes supplemental methods to store and formulate ADAS using E. coli as a model ADAS. E. coli ADAS with pBAD GFP plasmids are synthesized according to the methods in Example 12 and purified according to the methods in Example 13. They are then stored in various conditions to demonstrate their ability to reconstitute and survive.

a. Longitudinal Storage of ADAS E. Coli

ADAS are stored at 4° C. in isotonic buffer to maintain the structural integrity and chemical activity. Immediately prior to use, ADAS are rewarmed to 37° C.

### B. Longitudinal Storage of ADAS in Cold

ADAS are stored at 4° C. in isotonic buffer for 0, 30, 60, 90, or 180 days.

### C. Lyophilization and Reconstitution of ADAS

The ADAS are centrifuged and resuspended in the following solutions: 20% w/vol skim milk in growth media, 20% w/vol skim milk in water, 12% w/vol sucrose in a 50:50 mix of water and growth media, Reagent 18: Trypticase Soy Broth, 1.5 g; Sucrose, 10 g; Bovine Serum Albumin Fraction V, 5 g; Distilled water, 100 ml, and Reagent 20: Sucrose, 20 g; Bovine Serum Albumin Fraction V, 10 g; Distilled water, 100 ml. All solutions are filter sterilized through a 0.2 μm filter. The ADAS are then flash frozen in liquid nitrogen and lyophilized. The powder is stored for 0, 30, 60, 90, 180 days at 0° C., −20° C., and 25° C. At the end of the test period, the powder is reconstituted with water or LB broth. ADAS activity post reconstitution is evaluated using the methods to evaluate in Example 16.

### D. Freezing and Reconstitution of ADAS

The ADAS are centrifuged and resuspended in 10%, 20%, and 30% vol/vol glycerol and growth media. The ADAS are either flash frozen in liquid nitrogen, or slowly frozen in a Nalgene Mr. Frosty. The ADAS are stored at −20° C. and −80° C. for 0, 30, 60, 90, 180 days. At the end of the test period, the powder is reconstituted with water or LB broth.

ADAS activity post storage is evaluated using the methods to evaluate in Example 16.

### E. Spray-Drying and Reconstitution of ADAS

The ADAS are centrifuged and resuspended in 20% w/vol skim milk in growth media, 20% w/vol skim milk in water, or 12% w/vol sucrose in a 50:50 mix of water and growth media. The ADAS are then spray dried in a laboratory scale unit and collected. The powder is stored for 0, 30, 60, 90, 180 days at 0° C., −20° C., and 25° C. At the end of the test period, the powder is reconstituted with water or LB broth. ADAS activity post spray-drying is evaluated using the methods to evaluate in Example 16.

### Example 18. Assessing Similarity of Large ADAS to Parent Bacteria

This example describes large ADAS that are more similar to parent bacteria than regular ADAS.

a. Comparison of Large E. Coli ADAS with Other ADAS

E. coli ADAS are synthesized according to the methods in Example 12 and purified according to the methods in Example 13. Samples of ADAS, large ADAS, and parent bacteria are plated into well plates and assays are performed within 30 minutes of initial plating. Cytoplasmic composition is characterized using RNAseq (described in Example 14) to characterize the different RNA transcripts present in the different samples. Copy number of the different samples is evaluated using qPCR to quantify signal from single plasmids to those of a known copy number plasmid standard using a method based on existing protocols (Anindyajati et al. “Plasmid Copy Number Determination by Quantitative Polymerase Chain Reaction” Scientia pharmaceutica vol. 84,1 89-101. 14 Feb. 2016). Additionally, longevity of the three conditions are compared using the ATP activity assay described in Example 11. Samples of ADAS, large ADAS, and parent bacteria are all prepared and purified using methods previously described in Example 12-15. Full-transcriptome analysis is performed using RNAseq as described in Example 12 and differences in transcription magnitudes are noted.

### Example 19. Supplementary Methods for Demonstrating Higher Purity of Auxotrophic ADAS

This example describes ADAS made from auxotrophic parents that are purer after isolation than those made from non-auxotrophic parents.

A. Preparation of auxotrophic E. coli parental bacteria

ADAS with decreased parental bacteria contaminants are created using auxotrophic strains of E. coli, such as arginine synthesis knockout (argA) strain JW2786-1. E. coli ADAS are produced according to the methods of Example 12 but with arginine added to the media. The ADAS are purified using the methods in Example 13, and then stored in arginine-free media.

### B. Preparation of Auxotrophic Large E. Coli ADAS

To create auxotrophic large ADAS, an auxotrophic strain of E. coli, such as arginine synthesis knockout (argA) strain JW2786-1 E. coli are used. Large ADAS are produced according to the methods of Example 14, with the modification of the sbcB plasmid augmented with expression of the argA protein, allowing only the ADAS with sbcB to survive in the media. The ADAS are purified using the methods in Example 14.

### C. Demonstrating Increased Purity of Auxotrophic Large and Regular ADAS

Auxotrophic large and regular ADAS are prepared using the methods above. Non-auxotrophic ADAS and large ADAS are also prepared using the methods in Example 12, Example 13 and Example 14. ADAS purity is measured using the methods in Example 13.

### Example 20. Supplementary Methods for Determining Whether ADAS with Expressed ATP Synthase are Highly Active

ATP synthase expression and assembly are a tightly regulated process in all organisms. E. coli resist transcription with ATP synthase-containing plasmids as overexpression can be lethal to the cell. This example describes two strategies to synthesize ADAS with overexpression of ATP synthase.

a. Generation of E. Coli ADAS with Overexpression of Atpl

E. coli K12 lines are grown under normal culture conditions, total RNA is purified using TRIzol reagent (Invitrogen, Carlsbad, CA), and treated with 2 U of RNase-free DNase for 1 h. RT-PCR is performed on atpl, which is a gene involved in energy generation by ATP synthase, using the following primers: 5′-TCAGGCAGTCAGGCGGCTT-3′, atpl-F; 5′-TTACCCTTTGTTGTTAATTACAGC-3′, atpl-R as described by Chen et al., Adv Mat Res, 2014. The PCR conditions include an initial denaturation for 5 min at 96° C., followed by 15 cycles for 1 min at 96° C., 1 min at 55° C., and 1 min at 72° C. with a final extension of 7 min at 72° C. The PCR products are resolved on a 1.2% agarose gel. The intensity of expected bands is analyzed and compared by Bio-Rad software. To over-express the energy metabolism genes, the expression vector pET15b is employed. The PCR product of atpl gene is introduced into the plasmids pET15b to obtain the expression vector, pAtpl. The expression vector is electroporated into E. coli BL21 (DE3) and induced by addition of IPTG at OD550 0.4˜0.5.

E. coli ADAS are generated from this line as described in Example 12 and purified as described in Example 13.

B. Generation of E. coli ADAS with plasmids containing ATP synthase

The plasmid pBAD33.atp containing the ATP synthase cassette as described in Brockmann et al., J Bacteriol, 2013 is synthesized commercially and transfected into E. coli BL21 (DE3) using the methods described in the paper. ADAS are then produced from these cells following Example 12 with the removal of glucose and addition of 0.03% wt/vol arabinose to the media. The ADAS are then purified using the methods in Example 13.

C. Comparison of Activity of E. Coli ADAS and E. Coli ADAS with ATP Synthase

E. coli ADAS made from the above methods and E. coli ADAS without ATP synthase addition are synthesized and characterized using the methods in Example 15.

### Example 21. Assays for Increased Activity in ADAS with Suppression of ATP Synthase & Components

The & subunit (atpC) of the bacterial FoF1 ATP synthase is an intrinsic inhibitor of ATP synthesis/hydrolysis activity. Mutants defective in this regulatory domain exhibited no significant difference in growth rate, molar growth yield, membrane potential, or intracellular ATP concentration under a wide range of growth conditions and stressors compared to wild-type cells (Klionsky et al., J Bacteriol, 1984). In this example, E. coli cells are synthesized with inducible excision of the & subunit or made from a knockout strain.

a. Generation of E. Coli Parent Lines with Inducible Excision of ATP Synthase & Subunit

E. coli strain JW3709 or other strain with atpC knockout is obtained. A plasmid is constructed using a tetracycline-controlled transactivator (tTA) to inducibly express atpC in the absence of tetracycline (tet) by a commercial service. The construct is electroporated into the atpC knockout strain to allow for expression of atpC in the absence of tet and grown in media without tet. ADAS are synthesized from these cells using the protocols described in Example 12 and purified with the methods in Example 13. E. coli ADAS made from the above methods in the presence and absence of tet, E. coli ADAS made from strain JW3709, and regular E. coli ADAS are characterized using the methods in Example 15.

### Example 22. Highly Active ADAS with Modified Glycolysis Pathways

Since the glycolysis pathway is one of the ways ATP is synthesized, upregulating key enzymes may lead to more ATP in the cell. This example describes the creation of highly active ADAS from E. coli containing a plasmid expressing pfkA and tpi that has been shown to produce more ATP (see Shimosaka et al, Ag. Bio. Chem, 1981).

a. Synthesis of ADAS Expressing pfkA and Tpi

E. coli strain pLC16-4 from the E. coli Genetic Stock Center is grown and the plasmid pLC16-4 is extracted using a commercial plasmid purification kit. Electrocompetent E. coli C600 cells are prepared and electroporated with plasmid pLC16-4 following the protocol in Eppendorf protocol #4308 915.511. Briefly, cells are grown from a fresh overnight culture of E. coli at 37° C. in LB medium to a density of OD600 of 0.5-0.6. Cells are then put on ice, centrifuged chilled, resuspended in 0° C. water, washed in chilled water, and diluted to a concentration of 2×10¹¹ cells/mL. 40 μL of cells are then mixed with 10 μg of plasmid in water and electroporated. The cells are then immediately grown up for 30-60 mins at 37° C. in SOC medium, plated, and then grown up in standard culture conditions. E. coli ADAS from E. coli C600 containing and not containing the pLC16-4 plasmid are then prepared according to the protocols in Example 12 and purified according to Example 13.

B. Comparison of ADAS Overexpressing pfkA and Tpi with Regular ADAS

Activity of the E. coli C600 containing and not containing the pLC16-4 plasmid is measured using the protocols in Example 15.

### Example 23. Assays for Increased Activity in ADAS Synthesized in Specialized Culture Conditions

The culture conditions can dramatically change the growth, maturation, survival of cells in vitro (Wang D, Yu X, Gongyuan W. Pullulan production and physiological characteristics of Aureobasidium pullulans under acid stress. Appl Microbiol Biotechnol. 2013; 97:8069-77). This example describes screening of compounds and conditions that increase intracellular ATP. Non-exhaustively, E. coli are used with an initial emphasis on pH, oxygen concentration, and applied voltage. This represents a supplemental method for identifying new media additives to tune cellular ATP levels.

ADAS are synthesized and isolated using methods described in Example 12. After isolation, ADAS are cultured under various media conditions and the ATP production rate is measured using Seahorse (Agilent, ATP ASSAY kit) according to the manufacturer's instructions. 0.1 mM, 0.3 mM, 0.4 m, 1 mM, or 10 mM glucose is used as a baseline.

a. Synthesis ADAS in Low pH

The pH of the ADAS growth media is tuned using dropwise addition of citric acid and NaOH. Media of pH 4.5, 5, 5.5, 6, 6.5, 7, 7.5, 8, 8.5, 9, 9.5 used and intracellular ATP is measured immediately and after 1, 4, 6, 10 hours using the Seahorse ATP assay.

Our result shows an elevated ATP supply in conditions of lower pH, such as 4.5, 5, 5.5, 6, 6.5.

### B. Synthesis of ADAS in a Hypoxic Environment

To mimic hypoxic media conditions, media is incubated in various gas mixtures (1, 5, 7.5, 10,15, 20.95% O₂) for 24 hours prior to exposure to ADAS. E. Coli ADAS are then cultured in pre-conditioned media and intracellular ATP is measured immediately and after 2 hours using the Seahorse ATP assay. Our result shows that media with oxygen concentration of 1, 5, 7.5, 10, 15, 20.95% lead to increased intracellular ATP after 2 hours.

C. Synthesis of ADAS with Applied Alternating Electric Fields

An externally applied alternating electric field with an amplitude of intensities between 1-100 V/cm is applied to ADAS grown in standard LB media within a stimulation cell according to previously defined protocol developed for E. Coli culture (Zrimec et al, CELL. MOL. BIOL. LETT. Vol. 7. No. 1.

2002). In brief, the voltage is established between two parallel platinum electrodes (0.75 cm² active area, 2 mm apart) and a frequency generator (Metex MS-9150) is used to apply electric fields. The sample is kept at low temperatures (<5° C. during the stimulation) and the amplitude of the electrode voltage is varied from 0 V to 100 V with no DC component and a frequency ranging from 50 Hz to 1000 KHz. The result indicates de novo ATP production in ADAS is increased in samples at the highest voltage as measured by intracellular ATP using a BacTiter Glo assay (Promega) as prescribed by the manufacturer.

### Example 24. Assays for Increased Activity in ADAS with Removed Flagella and Other Non-Essential Features

To minimize the presence of non-essential structural features in E. coli ADAS, parent lines are synthesized with a simplified genome containing essential for growth, division and metabolism genes. Studies have shown that E. coli synthesized with such “essential” genomes not only survive and divide but can even provide some useful properties. Posfai et al., Science, 2006 showed that these strains show high electroporation efficiency and accurate propagation of recombinant genes and plasmids that are unstable in other, unmodified strains. These modified strains, therefore, not only result in E. coli ADAS devoid of unnecessary structures, but also have enhanced activity compared to those from unmodified parent lines.

Genome-based approaches are used to identify essential genes in E. coli genome, as described in Arigoni et al., Nature Biotech., 1998 and others. Briefly, a comparison of the E. coli genome to that of other bacteria allows key genes involved in growth, maturation and division to be identified. Systematic gene disruptions confirm the key genes involved in survival and division. In addition to this, genome architecture is analyzed using in silico methods to stabilize the minimal genomes. Deletion of large regions of the genome are done using CRISPR-Cas9 and phage-mediated deletions. In order to create scar-free deletions, the protocol adapted from Tear et al., Applied Biochem Biotech, 2015 is used. Additionally, the growth medium can mask or enhance the effects of such gene deletions, and these strains will need to be grown on a variety of media to fully test the effects (Ish-Am et al., PLOS One, 2015). A library of strains with varying numbers of non-essential genes that are necessary for specific functions (e.g. transporters, structure) is generated.

a. Generation of E. Coli ADAS with Flagella Deletion

E. coli MG1655 (CGSC 6300) is obtained from the E. coli Genetic Stock Center and is a wild-type strain that lacks the IS1 element in the flhDC promoter. These mutants are nonmotile and have deletions of various lengths beginning immediately downstream of an IS1 element located within the regulatory region of the flhDC operon, which encodes the master regulator of flagellum biosynthesis, FlhD4C2. ADAS are synthesized from these cells using the protocols described in Example 12 and purified with the methods in Example 13.

B. Generation of E. Coli Parent Lines with Fimbriae and TXSS Deletion

Multiple parent E. coli lines with disruption in genes encoding for fimbrial and TXSS proteins are generated using the Quick and Easy E. coli Gene Deletion Kit (Gene Bridges). This kit uses Red/ET Recombination, to target DNA molecules that are precisely altered by homologous recombination in E. coli which express the phage-derived protein pairs, either RecE/RecT, or Reda/RedB. RecE and Reda are 5′-3′ exonucleases, and RecT and Redβ are DNA annealing proteins. Briefly, PCR products targeting the gene of interest are inserted into the pRedET expression plasmid, and the E. coli strain to be modified is transformed as per manufacturer's directions. Red/ET expression is induced by addition of L-arabinose and a temperature shift. Red/ET-mediated recombination disrupts the target locus by insertion of a repeat cassette and results are verified using PCR. ADAS are synthesized from these cells using the protocols described in Example 12 and purified with the methods in Example 13.

C. Comparison of E. Coli ADAS with and without Flagella, Fimbriae, and TXSS

ADAS are generated from parent lines above, and activity is measured using the methods in Example 15.

### Example 25. Assays for Increased Activity in ADAS with Photosynthetic Capabilities

This example describes the synthesis of highly active ADAS with the ability to convert light into PMF or ATP through the addition of a plasmid expressing proteins that can harvest light energy, with various rhodopsins as a model protein.

a. Synthesis of Proteorhodopsin Expressing E. Coli ADAS

E. coli lines expressing proteorhodopsin (PR) are created using the protocol described by Walter et al., PNAS, 2007. Briefly, a plasmid containing the SAR86 y-proteobacterial PR-variant in E. coli cells is expressed under a T7-promoter. Cells are grown in T broth, and PR expression is induced with 1 mM isopropyl β-D-thiogalactoside. ADAS are synthesized from the PR-containing E. Coli cells and non-PR containing E. coli cells using the methods in Example 12 and purified according to the methods in Example 13, with some of the ADAS purified under exposure to 70 μmol photon/(m\hat{ }2 s) light and some purified in dark.

E. coli cells expressing PR and genes required to retinal are created following an adaptation of the protocol in Kim et al, Microb Cell Fact, 2012. Briefly, the pAcyc-RDS plasmid in the paper is synthesized commercially and transfected into chemically competent E. coli BL21 (DE3) following the manufacturer's instructions (NEB, protocol C2527). Briefly, cells are thawed on ice slowly, 1 pg-100 ng of plasmid DNA is added to the tube of cells, the cells are flicked 4-5 times, rested on ice for 30 minutes, heat-shocked at 42° C. for 10 s, placed on ice for 5 mins, SOC is added to the mixture, and the cells are cultured for 60 mins at 37° C. Cells are then plated on chloramphenicol plates for selection and grown up in LB media containing chloramphenicol. ADAS are synthesized from the PR-containing E. Coli cells and non-PR containing E. coli cells using the methods in Example 12 and purified according to the methods in Example 13, with some of the ADAS purified under exposure to 70 μmol photon/(m\hat{ }2 s) light and some purified in dark.

### B. Comparison of Proteorhodopsin Containing ADAS and Non-Proteorhodopsin Containing ADAS

Activity of the E. coli containing and not containing the PR and cultured in dark or light is measured using the protocols in Example 15.

### C. Comparison of Proteorhodopsin Containing ADAS and Retinal and Proteorhodopsin Containing ADAS

Activity of the PR-containing E. coli with or without the retinal and cultured in dark or light is measured using the protocols in Example 15.

### Example 26. Supplemental Methods for Creating E. Coli ADAS with Stable Nucleic Acid and Protein Payloads

Stable intracellular RNA (e.g. miRNAs, siRNAs, and RNA aptamers) within ADAS is an important step towards creating new RNA delivery technologies as the nucleic acids can be readily degraded by endonucleases in the cytoplasm. Additionally, upon entering the host cell, tRNA-RNA scaffolds are then precisely separated into a 5′ tRNA and a 3′ pre-miRNA upon cleavage by cellular tRNase Z, which functions to define 3′ end of cellular tRNAs. Utilizing recently developed protocols (RNA. 2015 September; 21 (9): 1683-1689.) TRNase Z can be used to cleave guide RNA-tRNA fusions. This example describes a scaffold of non-coding RNA derived from tRNA is expressed within E. Coli as an illustrative example, however, other nucleic acids, proteins, and molecules could also be stabilized within ADAS using similar scaffold-based approaches.

a. E. Coli ADAS with tRNA Stabilized Recombinant RNA Payloads

Briefly, E. coli ADAS are produced and purified using methods described by Example 12 and 13. Based on previously developed protocols, two types of tRNA are used: humantRNALys3 and E. coli tRNAMet (Nat. Methods, Ponchon 2007). Both have been well characterized and expressed recombinantly. In principle, any structured RNA terminated with a stem motif could be included. These include aptamers, lncRNA, and ribozymes. In brief, a plasmid containing an RNA sequence of interest that is flanked on each side by a tRNA insert is created. In this example, mRNA encoding for GFP is used.

After ADAS are synthesized containing tRNA-mRNA construct, half-life of the RNA and the protein is measured. Briefly, cells are transferred to well plates and lysed using a lysis buffer at different time points and RNA SEQ is performed at various time points including 1 min, 10 min, 50 min, 100 min, 200 min, 500 min, 1000 min, 2000 min, and up to 1000000 min, and relative abundance is used to evaluate stability. Furthermore, GFP expression is observed at the same time points by lysing a defined number of the cells and measuring fluorescence through a plate reader.

### Example 27. Increasing Cargo Stability by Removal of Ribonucleases from E. Coli ADAS

RNase E is the universal initiator of RNA degradation, and chromosomal deletion in E. coli disrupts the colony-forming ability (CFA) of E. coli strain. This example shows that by putting RNAse E under control of a conditional promotor, enhanced protein and RNA stability can be achieved.

a. Synthesis of E. Coli Parent Lines with RNase E Deletion

E. coli parent line with a chromosomal deletion of Eco-rne, which encodes for RNase E is generated by inserting a plasmid-borne Eco-rne gene under the control of an araBAD promoter, is used to enable E. coli to synthesize RNase E in the presence of arabinose. A method of plasmid integration into E. coli is derived from previous protocols (Tamura et al. PLOS One, 2017).

### B. Synthesis of RNase-Free ADAS

ADAS are generated from parent E. coli lines lacking chromosomal Eco-rne. Prior to ADAS synthesis and purification, as described in Examples 12 and 13, parent lines are grown in arabinose-free medium for 1, 2, 3, 4, 8, 12, 24, 48 hours or 3, 4, 5, 10, 14 days to allow degradation of residual RNase E.

C. Metabolic Activity and mRNA Stability Index

RNase-free and unmodified ADAS are grown in LB broth, and ATP production is measured using a Seahorse XF (Agilent, ATP Rate assay) at 2 h, 12 h, 1 d, 7 d and 30 d. RNase free ADAS shows comparable levels of metabolism to unmodified ADAS. Both ADAS lines express GFP after transformation with a plasmid containing GFP. GFP expression is measured using fluorescence imaging and mRNA levels are measured using RNAseq.

D. Inhibition of Ribonucleases from E. Coli ADAS

To inhibit the degradation of RNA, which is essential for the generation and maintenance of function within E. coli ADAS, existing ribonucleases can be inhibited. For this, small molecule inhibitors of RNase E are used. RNAse E is considered the global initiator of RNA decay in E. coli and forms the platform for the degradasome complex. The N-terminus forms the catalytic portion of this enzyme, and as such, can be inhibited by a small molecule.

Small molecule screening methods developed previously for intact E. coli (Kime et al., Sci. Rep., 2015) are adapted to assess the inhibition of RNAse E in E. coli ADAS and the best candidates for inhibition are tested in vitro. The sample methods described above where GFP is quantified over time are used to calculate the stability index.

### Example 28. Synthesis of a B. Subtilis ADAS with a Tat Signal Peptide

This example describes ADAS that can be used to secrete peptides through channel-like secretion systems, and highly active B. subtilis ADAS that are capable of secreting more proteins over longer periods of time.

a. Synthesis of B. Subtilis ADAS Secreting GFP

B. Subtilis ADAS are synthesized using modified methods (Feucht et al. Microbiology. 2005 June;151 (Pt 6): 2053-64.) with “cargo” that is tagged for export into the cytoplasmic space. Alternatively, B. Subtilus ADAS are created through a targeted divIVA1 mutation (JH Cha, GC Stewart-Journal of bacteriology, 1997-Am Soc Microbiology), but otherwise collected, purified, and characterized as previously described in Example 12 and Example 13. The Tat secretion system signal is fused onto a target protein to induce production and export from the ADAS cytoplasm into the extracellular space. A TAT signal peptide is incorporated into a green fluorescent protein (GFP) is fused to the PhoD Tat signal peptide and excreted out of B. Subtilus using modified protocols described previously (Wickner et al. Science 2005, B. C. Berks, Mol. Microbiol. 22, 393, 1996). The extracellular GFP is measured using fluorescence microscopy.

### B. Synthesis of Highly Active B. Subtilis ADAS

Highly active B. subtilis ADAS are synthesized by adapting the methods from Examples 20-25, for B. subtilis through using the B. subtilis versions of the genes, promoters and plasmids identified. The TAT-signal fused GFP plasmid from part a) is also transfected into the B. subtilis, leading to secretion of GFP. The ADAS synthesized from part a) and part b) are tested for protein secretion at 1, 2,4, 8, 12, 24, 48, 36, and 72 hours.

### Example 29. Immunomodulatory ADAS for Tumor Suppression

This example describes highly active ADAS that are capable of containing and delivering cargo that has a specific effect on the immune system of the host. As a model that is not a sole or limiting embodiment of this concept, a highly active B. Subtilis ADAS that secretes specific cyclic dinucleotides (CDNs) is created. In particular, the ADAS secretes specifically bacterial nucleotides c-di-AMP that engage the STING pathway via the RECON (REductase Controlling NF-κB) of mammalian cells (Mol Cell. 2008; 30:167-78).

B. subtilis ADAS are synthesized as previously described in Example 28. Briefly, using modified assays described in (MacFarland et al: Immunity. 2017 Mar. 21; 46 (3): 433-445.), human peripheral blood mononuclear cells are exposed to (1E6, 1E7, 1E8, 1E9, 1E10) B. Subtilis ADAS per mL concentration for (10, 20, 30, 60, 120, 1200 min). Next, the cells are lysed and PCR is utilized to determine the fold increase in immunostimulatory genes, such as IRF3-, IFN-, and NF-κB-dependent genes in the hPBMCs.

### Example 30. Synthesis of Pseudomonas putida ADAS and Highly Active ADAS

This example describes the synthesis of ADAS and highly-active ADAS from a T6SS expressing and plant commensal bacteria, using P. putida as a model organism.

a. Synthesis of Pseudomonas putida ADAS

Pseudomonas putida KT2440 is obtained from ATCC and cultured according to manufacturer's instructions. A plasmid which overexpresses P. putida ftsZ under an inducible pLac-1k-J23107 promoter (Cook et al., J. Ind. Microb. Biotech., 2018) is constructed from the pBBR1MCS-2 backbone by a commercial vector generation company. P. putida ADAS parent lines are synthesized by electroporation of the plasmid DNA into the bacteria following the protocol described in Chen et al, Chin. J. Appl. Env. Bio., 2010. Briefly, P. putida KT2440 is grown to OD600 of 0.6-0.75, pelleted at 4° C., washed in 3 mmol/L HEPES, electroporated in a Bio-Rad Gene Pulser or equivalent machine, reconstituted with SOC media, and incubated at 30° C. The cells are then plated on LB Kanamycin agar for selection and grown according to manufacturer's instructions with the addition of IPTG to induce ADAS formation. ADAS are then purified using the methods in Example 13.

B. Synthesis of Highly Active Pseudomonas putida ADAS Through Removal of Flagella and T3SS

Either P. putida EM42 and EM383 are obtained or another flagella-free derivative of P. putida KT2440 is synthesized following the procedure in Martinez-Garcia et al, Microb. Cell. Fact., 2014 and Martinez-Garcia et al, Environ. Microbiol., 2013. Briefly, the region 750-bp upstream (TS1) and 816-bp downstream (TS2) of the pp 4329 and pp 4297 genes is PCR amplified, and the TS1 and TS2 fragments are ligated through overlap PCR (see Horton et al., Gene, 1989). The complete TS1-TS2 fragment is digested with EcoRI and BamHI and ligated into the plasmid pEMG (GenBank: JF965437.1) to generate plasmid pEMG-flagella. The plasmid is transformed into E. coli DH5a Apir, and electroporated into P. putida KT2440 prepared with the I-Scel meganuclease under 3-methylbenzoate promoter as described in Martinez-Garcia and de Lorenzo, Meth. Mol. Bio., 2012. The positive co-integrates are selected through PCR amplification of the TS1-TS2 fragment and resolved through induction of the I-Scel enzyme, derived from the pSW-I plasmid using 15 mM 3-methylbenzoate. The culture is plated onto LB-Ap500 agar plates and the deletion is confirmed by PCR.

ADAS are produced from the resultant strain using the methods described in part a).

### C. High-T6SS Expressing ADAS

The K1 T6SS as indicated in Bernal et al, ISME J., 2017 is cloned into a pBBR1 origin vector with amp resistance via commercial gene synthesis with its natural promoter and with the natural promoter replaced by a pLac-1k-J23107 promoter (Cook et al., J. Ind. Microb. Biotech., 2018). The plasmid is then transfected into an ADAS-producing P. putida line using the methods described above and grown in ampicillin and kanamycin containing LB media and ADAS are purified using the methods from Example 13.

### D. Determining Number of T6SS on ADAS

A quantum-dot labeled antibody against VgrG trimer is used to label active T6SS secretion systems on the surface of the ADAS. This is imaged by confocal microscopy and active T6SS are counted manually by the presence of punctate spots. The average distance between T6SS on the membrane is computed and squared and is treated as the surface area covered per T6SS.

### Example 31. T6SS-Expressing ADAS as an Antimicrobial and a Plant Protectant

Pseudomonas putida is a bacterium found in plants that has been demonstrated to have plant protective activities due to T6SS attack of potential plant pathogens. This example describes P. putida ADAS that are capable of lysing bacteria using T6SS systems using E. coli as a model bacteria and X. campestris as a model plant pathogen.

a. Using Pseudomonas putida ADAS to Kill Escherichia coli

Pseudomonas putida ADAS and highly-active ADAS are synthesized using the methods from Example 30. The ADAS are used to lyse E. coli cells using the protocol adapted from Bernal et al., ISME J., 2017. Briefly, competition assays are performed on LB plates. E. coli are cultured to an OD600 of 1 in PBS and mixed in a 1:1 ratio with P. putida ADAS on the plates. Plates are also grown without any P. putida ADAS. The plates are incubated at 30° C. for 5 h and colony-forming units are counted on antibiotic selection.

B. Using Pseudomonas putida ADAS to Kill Xanthomonas campestris.

Pseudomonas putida ADAS are used to lyse X. campestris cells using the same assay as described in part b), with the exception that X. campestris cells are cultured for 24 hours on the plate. The same assay is run to demonstrate that P. putida ADAS reduces the number of X. campestris colonies and highly active ADAS reduce the number more.

C. Using Pseudomonas putida ADAS as a Plant Protectant

P. putida ADAS are used to protect plants from damage following the protocol adapted from Bernal et al., ISME J., 2017. Briefly, in planta competition assays are carried out by infiltration of bacteria into Nicotiana benthamiana leaves. Overnight cultures of Xanthomonas campestris are adjusted to OD600 of 0.1 in PBS. P. putida ADAS are mixed with the bacteria in a 1:1 ratio. Approximately 100 UL volume is infiltrated on the reverse of a 1-month-old leaf and the infiltration area is marked. After 24 h of incubation in a plant chamber (23° C., 16 h light), colony-forming units are determined. A section of the leaf from the infiltration area is cut out, homogenized in PBS, and subsequently serially diluted. The leaves are visualized by fluorescence microscopy. The evaluation of necrosis is based on the coloration of the leaves following Katzen et al., J. Bacteriol, 1998 using visible changes in the tissue color of the leaf, which can shift from green to yellowish (chlorosis), yellowish to brownish and blackening of the leaf (necrosis), up to complete rotting of the leaf at later stages.

### Example 32. Serratia marcescens ADAS as a Model Antifungal ADAS

S. marcescens is a rod-shaped, gram negative bacteria which has recently been demonstrated to kill fungal cells via T6SS delivery of the effectors Tfe1 and Tfe2 (Trunk et al., Nat Microb, 2018). This example describes using S. marcescens ADAS to reduce the fitness of fungi.

a. Synthesis and Purification of S. Marcescens ADAS

A plasmid containing the Pseudomonas ampR promoter PampR and the S. marcescens ftsZ gene is introduced into the pUC19 backbone with a synthetic RBS in a manner similar to the PQY38 plasmid in Yan and Fong, Appl. Microbiol. Biotech., 2017. The plasmid is introduced into S. marcescens Db10 from the Caenorhabditis Genetics Center using electroporation following the methods in Yan and Fong, Appl. Microbiol. Biotech., 2017. Briefly, the cells are washed cold in cold water via a chilled centrifuge, resuspended in cold water, plasmid DNA is added, and the mix is electroporated. Afterwards, cells are grown in 1 mL SOC medium at 30° C. for 60 mins and then plated on LB amp agar plates. S. marcescens cells with ftsZ are then grown in M9 medium 0.1% yeast extract, 2% glucose, and 200 mg/L ampicillin at 30° C. ADAS are then purified using the methods in Example 13.

### B. Demonstrating Ability of S. Marcescens ADAS to Kill Fungi

Antifungal activity is measured using a co-culture assay similar to one from Trunk et al., Nat Microb, 2018. Parent lines, ADAS and target cells are normalized using optical density. Target cells and either parent lines, ADAS, or control E. coli are mixed at a 1:1 volume ratio and 12.5 μl of the mixture is spotted on solid SC+2% glucose media. Cultures are incubated for 2, 7, and 24 hours, and surviving fungal cells are quantified by serial dilution and viable counts on streptomycin supplemented YPDA media to remove bacteria and ADAS. Fungal cells are also visualized using DIC in real-time to measure cell growth and division. E. coli are used in co-cultures as a negative control. Both S. marcescens parent lines and ADAS are able to kill fungal cells following 7 hours of co-culture.

### Example 33. Delivering Proteins to Human Gut Epithelial Cells Via Overexpressed T3SS

The ability to deliver intracellular proteins to mammalian cells could enable a variety of applications, in human therapeutics. This example describes that heterologous protein can be delivered to human gut epithelial cells using ADAS T3 secretion systems. This model embodiment could be applied to any number of animal and plant applications in which intracellular protein could have a marked effect on the target organism.

In this experiment, human gut epithelial cells are exposed to various concentrations of ADAS (1E6, 1E7, 1E8, 1E9, 1E10 ADAS per mL) and intracellular GFP is measured in the epithelial cells using fluorescence microscopy. The number of cells positive for GFP is divided by the total number of cells for various conditions to calculate delivery and dosing efficiency.

Caco-2 mammalian cell lines are cultured in accordance to ATCC. The cells are modified to express truncated GFP (tGFP) as described by Huang and Bystroff, Biochemistry, 2009. This truncated form of GFP is expressed and folded correctly but is not fluorescent until the missing β-strand is supplied. E. Coli and ADAS are produced and purification as described in Example 12 and 13, respectively. In brief, they are cultured at 37° C. on Corning® tissue culture flasks (T-25, 75, T-150, or T255, catalog #430641) with ATCC's custom Eagles Minimum Essential Media (Catalog No. 30-2003) containing non-essential amino acids, 2 mM L-glutamine, 1 mM sodium pyruvate, and 1500 mg/L sodium bicarbonate supplemented with fetal bovine serum at 20%. To harvest Caco-2 cells for the delivery assay, cells are washed with PBS without divalent cations (Ca2+ and Mg2+) and 2-3 mL of 0.25% (w/v) Trypsin and 0.05 mM-EDTA solutions are added to cell surface for 10-15 minutes and enzyme activity is quenched with 6-8 mL of complete growth media. After harvesting, the cells are counted using an automated cell counter (Countess II, Thermo Fisher) and live/dead stains (Calcein-live, ethidium homodimer-dead).

a. Preparation and Purification of E. Coli ADAS with T3SS from Shigella SPI-1

E. Coli ADAS are prepared using methods described in Example 12. To increase the abundance of T3SS on ADAS surface, a plasmid coding for HilA is introduced. HilA regulates SPI-1 T3SS which is adopted from an existing protocol (Bajaj et al. Molecular Microbiology (1995) 18 (4), 715-727). ADAS are prepared using methods described in Example 13. T3SS are visually inspected and counted using immunofluorescence microscopy and TEM. After comparison of isolated highly-pure T3SS expressing ADAS with non-purified T3SS expressing ADAS.

### B. Protein-Secretion System Tag Fusion

To enable protein secretion using a functional T3SS, GFP β-strand is fused to the first 104 amino acids of the T3SS secretion tag, SopE using a previously described plasmid design methodology (Evans, et. al. J. Virol., February 2003, p. 2400-2409). The plasmid is expressed in parental E. coli and subsequent ADAS contain the truncated SopE-GFP hybrid.

### C. Protein Delivery Assay

Prior to exposure to ADAS, cells are cultured 1 or 3 or 5 days post-confluency to ensure reproducible cell maturity and polarization. For each sample, ADAS are added to the confluent epithelial sample and incubated at 37° C. for 2 hrs. Efficiency of ADAS delivery is calculated by dividing the total number of fluorescence cells by the total number of cells in culture and compared between the various conditions.

### Example 34. Supplemental Methods for Targeted Delivery of E. Coli ADAS with T3SS Systems

Targeted delivery is important in drug delivery. This example describes using nanobodies as a model targeting agent for ADAS. Nanobodies are the smallest known functional antibody fragments, and recent work has shown that these can be expressed on the surface of E. coli (Salema and Fernandez, Microb Biotechnol, 2017). Surface nanobodies can efficiently bind to target proteins and can be used to generate specificity to individual cell types.

a. Synthesis of E. Coli Parent Lines Containing EGFR Nanobodies

Parent E. coli lines are synthesized expressing surface nanobodies for the epidermal growth factor (EGFR) as described by Salema et al., MAbs, 2016. Briefly, the sequence coding the nanobody for EGFR (TYNPYSRDHYFPRMTTEYDY) is cloned into the sites of the E. coli display vector pNeae2, which fuses the nanobody to the C-terminal of intimin polypeptide Neae allowing for surface expression. The plasmid pNeae2 (CmR) is a derivative of the pNeae vector, encoding Intimin residues 1-659 (from EHEC O157: H7 strain EDL933stx-) followed by the E-tag, the hexahistidine (His) epitope, and a C-terminal myc-tag (EQKLISEED).

Bacteria carrying plasmids with nanobody are grown at 30° C. in LB broth on agar plates with the appropriate antibiotic for plasmid selection. LB plates and pre-inoculum media prior to induction contained 2% (w/v) glucose for repression of the lac promoter. The preinoculation cultures are started from individual colonies (for single clones) or from a mixture of clones (in case of libraries), freshly grown and harvested from plates, diluted to an initial OD600 of 0.5, and grown overnight under static conditions. For induction, bacteria (corresponding to an OD600 of 0.5) are harvested by centrifugation (4000×g, 5 min), and grown in the same media with 0.05 mM isopropylthio-β-D-galactoside (IPTG), but without glucose for 3 h with agitation (160 rpm), unless indicated otherwise.

B. Synthesis of ADAS with EGFR Nanobody Expression

ADAS are synthesized from these parent lines as described in Example 12 and loaded with cargo (protein and gene delivery for GFP) as described above.

C. Specificity Assay for ADAS with EGFR Nanobody

Caco-2 (human epithelial colorectal adenocarcinoma) and A-431 (epidermoid carcinoma) cell lines are co-cultured according to standard cell culture protocols provided by the manufacturer. Co-cultures are then incubated with modified parent lines or ADAS carrying cargo (GFP) as described above. FACS is used to separate A-431 and Caco-2 cells, and to quantify the percentage of target cells expressing GFP. Fluorescent microscopy is also used to quantify fluorescence levels in individual cells to measure the number of protein subunits injected or amount of protein expression due to transfection. Microscopy is also used to quantify GFP expression in cell type based on cell morphology to differentiate between Caco-2 and A-431 cells along with immunocytochemistry to label for cell-specific markers.

### Example 35. Synthesis of Xanthomonas citri ADAS for T3SS Delivery to Plants

This example describes creating ADAS from plant pathogenic bacteria and using their natural delivery functions with Xanthomonas citri as a model organism. T3SS found in plant pathogens forms the basis for an efficient protein delivery system to plant cells. Since the T3SS effectors from X. citri are not well-characterized, an assay to measure biofilm formation is used to assess T3SS ability in X. citri ADAS. The effectors can then be replaced with reporter proteins (e.g. GFP) to assess ability to transfer heterologously expressed proteins via T3SS.

a. Synthesis of T3SS-Deficient X. Citri

T3SS is necessary in X. citri for biofilm formation during infection and increases virulence. A T3SS-deficient X. citri is created by creating a mutant with deficiency in the hrpB operon which encodes for key T3SS proteins (described by Dunger et al, Plant Pathol, 2005). Primers used are 5′-GAACTGGGCGGGAAGAACGACGAG-3′ and 5′-GCCGCCGCCGAAGAAGTGATG-3′. Genomic DNA (100 ng) is used as the template in a 50-μL reaction mixture. PCR is carried out in an Eppendorf thermal cycler, with denaturation at 94° C. for 5 min, followed by 30 cycles of 94° C. for 30 s, 62° C. for 40 s, and 72° C. for 1 min, with a final extension at 72° C. for 5 min. PCR-amplified products are analyzed in 0.9% agarose gels. Southern blot hybridizations are performed using Xachrp-mutant DNA fragments produced by digestion with BamHI, fractionated by electrophoresis and then transferred to Hybond-N membranes. Hybridization and detection protocols are performed using amplified hrp840 bp PCR products labelled with [α-32P]dATP.

The hrpB mutant is constructed by plasmid integration. The amplified product is cloned into the suicide vector pK19mobGII digested with Smal, yielding pKmobB and pKmobF, respectively. Plasmids are transferred to X. axonopodis pv. citri by biparental mating from the broad host-range-mobilizing E. coli strain S17-1. Bacterial mixtures are spotted onto Hybond-C membranes, placed on Nutrient agar and incubated for 48 h at 28° C. The membranes are then washed, and the bacteria transferred to selective medium. Xanthomonas axonopodis pv. citri mutant strains are selected by the vector-encoded antibiotic resistance (Km) and verified by Southern hybridization.

### B. Synthesis of X. Citri ADAS

To create X. citri ADAS, X. citri (hrpB mutants and untransformed) are transfected with a plasmid that overexpress ftsZ protein under the arabinose promoter (Lacerda et al, Plasmid, 2017), similar to Example 12. The ftsZ gene sequence is from Kopacz et al., MicrobiologyOpen, 2018. The primer sequences used are: forward-GAGCCCATGGCACATTTCGAACTGATTGAAAAAATGGCTCCCAACGCGGTCATCAAGG; reverse-AGTTCATATGCGACGCAGCCGACGCTCCTCAG. Plasmids are synthesized commercially by Thermo Fisher. Transfection is carried out using the process described above. Over time, the selected colonies proliferate and produce ADAS continuously.

C. Ability of X. Citri ADAS to Form Biofilm on Citrus sinensis

T3SS is essential for biofilm formation by X. citri. ADAS from hrpB mutant and normal strains are used in an infection assay in C. sinensis plants. All plants are grown in a growth chamber with incandescent light at 28° C. with a photoperiod of 16 h. ADAS are purified to an OD600 of 1, and resuspended in 10 mM MgCl2 at 104 to 107 cfu/ml. These are infiltrated onto leaves with needleless syringes. Cankers are counted from 20 orange leaves inoculated with the different strains and the areas of the counted leaves are measured from digitalized images using Adobe Photoshop software. ADAS from T3SS-expressing X. citri show significantly more canker formation than those from T3SS-deficient parent lines.

### Example 36. Synthesis of Agrobacterium ADAS as a Plant Transformation Agent Using T4SS

This example describes creating ADAS that can deliver payloads, such as a model DNA plasmid, with T4SS. Agrobacterium tumefaciens are used as a way to transform plants using their native T4SS systems. This allows non-replicative agrobacterium to be deployed in a spray modality in large fields without risk of escape.

a. Transfection of Agrobacterium with a Plasmid

For Nicotiana Benthamiana transfection, plasmids with antibiotic resistance genes are introduced into Agrobacterium strain GV3101 using the freeze-thaw method and materials as described in the Agrobacterium Transformation Kit (MPbio). Briefly, Agrobacterium is grown to an OD600 of 1.0 to 2.0, resuspended in a transformation solution, mixed with the plasmid DNA, submerged into liquid nitrogen for 1 minute, submerged in a water bath at 30° C. for 5 minutes, and regrown in Luria-Bertani medium with the selection marker on the plasmid.

For tomato transfection, plasmids with antibiotic resistance genes are introduced into Agrobacterium strain LBA4404 ElectroMAX cells (Thermo Fisher) using the methods described by the manufacturer's instructions. Briefly, the agrobacterium is thawed on wet ice and mixed with purified plasmid DNA free of salts, ethanol, and other contaminants. The mixture is electroporated, immediately mixed with room temperature medium, and regrown for 3 hours.

The cells from either protocol are then diluted and plated in agar with the antibiotics necessary to select. The next day, or when colonies are visible, they are selected for sequencing. After confirmation of plasmid identity, the proper colonies are grown overnight shaking in culture medium (specified by the manufacturer's instructions) containing the selection markers necessary.

### B. Synthesis of Agrobacterium ADAS

A plasmid containing the agrobacterium ftsZ on a pSRKGm backbone (Khan et al, Appl. Environ. Microbiol., 2008) is synthesized commercially. It is then transfected into agrobacterium using the methods described in part a) to form A. tumefaciens ADAS parent lines. The parent line is then grown according to manufacturer's instructions with the addition of IPTG and ADAS are purified using the methods in Example 15.

### C. Synthesis of Large Agrobacterium ADAS

A plasmid containing exonuclease V on a pSRKGm backbone (Khan et al, Appl. Environ. Microbiol., 2008) is synthesized commercially. It is then transfected into agrobacterium using the methods described in part a) to form A. tumefaciens ADAS parent lines. The parent line is then grown according to manufacturer's instructions with the addition of IPTG and ADAS are purified using the methods in Example 13.

D. Synthesis of a. Tumefaciens ADAS with GFP Payload

The plasmid pLSLGFP.R referenced in Baltes et al., The Plant Cell, 2014 is used. The plasmid pLSLGFP.R is transfected using the methods shown in part a). A. tumefaciens ADAS are synthesized using the methods in part b) or c).

E. Transfection of Plants with Agrobacterium ADAS with GFP Payload

Tobacco (Nicotiana tabacum var Xanthi) plants are grown at 21° C. with 60% humidity under a 16-h-light and 8-h-dark cycle. Leaves (fully expanded upper leaves) from 4- to 6-week-old tobacco plants are used for ADAS transformation. One leaf per plant is infiltrated. Leaves from tobacco plants are infiltrated with Agrobacterium using a 1-ml syringe. Immediately following infiltration, plants are watered and covered with a plastic dome to maintain high humidity. Plastic domes are removed ˜24 h after infiltration. Plants are imaged with a fluorescent reader and the percentage of leaf with visible GFP is measured.

### Example 37. Measuring ADAS Secretion Efficiency

In order to measure secretion efficiency of ADAS, the ability to synthesize and secrete endogenously and heterologously expressed proteins are measured.

a. Parent E. Coli Lines that Synthesize GFP

E. coli lines containing vectors carrying GFP and ampicillin resistance are ordered from ATCC (ATCC® 25922GFP™) and cultured according to manufacturer's directions.

### B. ADAS Ability to Synthesize Molecules

ADAS are generated from E. coli parent lines expressing GFP as well as from native E. coli. Fluorescent measurements are made from GFP-expressing E. coli parent lines as a positive control and used as a baseline to measure GFP intensity from ADAS. ADAS are synthesized according to Example 12, plated onto a 96-well plate and fluorescence is measured using a plate reader. Since ADAS do not need to synthesize proteins required for growth or division, both ADAS generated are more efficient at protein synthesis and exhibit higher GFP intensity compared to parent lines. To measure intensity at a cellular level, E. coli parent lines and ADAS are fixed and mounted on glass slides using standard cell preparation protocols. These are imaged using a confocal microscope with fluorescence imaging. These images are used to measure fluorescent intensity per square unit of area and are used for direct comparison with parent lines.

### C. ADAS Ability to Secrete Molecules

E. coli use T1SS-mediated lipase secretion to facilitate infection. T1SS contains ABC transporters which recognize specific C-terminal sequences in the secreted protein. Parent E. coli lines are generated expressing native T1SS along with endogenous lipases, as well as another line transfected with a plasmid containing GFP fused with the C-terminal sequence for transport as described by Chung et al., Microb Cell Fact, 2009.

Briefly, lipase ABC transporter domains (LARDs) are designed for the secretion of fusion proteins. The LARDs included four glycine-rich repeats comprising a β-roll structure and are added to the C-terminus of test proteins. Either a Pro-Gly linker or a Factor Xa site is added between fusion proteins and LARDs. Before the secretion of GFP fusion protein is analyzed, the expression of the GFP-fusion proteins is checked. These proteins are expressed in E. coli and their expected sizes are confirmed by Western blotting (Rockland Inc., PA, catalogue #KCA215). In addition, the fluorescence of GFP is demonstrated. Representative colonies of E. coli expressing these proteins are viewed under ultraviolet (UV) light. The secretory phenotype could be traced via lipase activity. E. coli is cultivated on tributyltin agar to detect the secretion of TliA-fusion proteins. When GFP fusion proteins are also detected with antibody against GFP, the same bands detected by the antibody against LARD are detected in the cell and supernatant.

Methods adapted from Chung et al. are also used to measure secretion and are outlined below. ADAS are generated from E. coli parent lines outlined in Example 12.

To measure lipase secretion by ADAS, ADAS are plated onto a solid medium (LAT: LB broth, 1.5% Bacto Agar, 0.5% tributyltin). Secreted lipase causes a halo to develop around the colony as a typical phenotype. ADAS develop haloes faster than parent lines. In addition to this, lipase activity is measured spectrophotometrically by adding p-nitrophenyl palmitate (pNPP) as a substrate dissolved in ethanol and Tris-HCL. 50 μL supernatant from ADAS and parent line cultures is added to 200 μL of the substrate and absorbance of 420 nm is measured using a plate reader. Activity is measured using by the increase of optical density.

Since lipases are endogenously expressed proteins, GFP is used to measure secretion of heterologously expressed proteins. ADAS and parent lines are grown in LB broth, and supernatant from the broth is collected to measure GFP expression. Standard immunoblot (Rockland Inc., PA, catalogue #KCA215) is used according to manufacturer's instructions with a GFP-specific antibody to measure GFP secretion.

### Example 38. Non-Rod-Shaped Nanoparticle-Containing ADAS (Magnetospirillum Magneticum)

This example describes that ADAS can be synthesized from non-rod-shaped bacteria and may contain larger structures, such as nanoparticles.

a. Preparation of M. Magneticum ADAS

To create ADAS, M. magneticum are transfected with a plasmid that overexpress ftsZ protein (Q2W8K6_MAGSA) under the T7 promoter. Plasmids are synthesized commercially by Thermo Fisher. Transfection is carried out using standard bacteria transfection processes (See Thermo Fisher Molecular Biology Handbook). In short, competent cells are plated on room temperature agar plates. 0.5-2 ng/ml of DNA are added to the competent cells within a vial and incubated for 20-30 minutes. Each tube is then heat shocked by placing the tube in 42° C. water bath for 30-60 seconds to create transient pores in the cell surface. Cells are then plated on agar gels that have been preloaded with selective antibiotic and grown overnight. Only bacteria that have been transfected with the plasmid survive. A single colony is picked and cultured at 37° C. with continuous shaking at 120 rpm in LB medium containing 50-100 μg/mL of ampicillin. Over time, the selected colonies proliferate and produce ADAS continuously. Alternatively, a protocol similar to that used in Example 14 is used to prepare large ADAS.

In addition to this, deletion of a ftsZ-like gene results in the production of superparamagnetic magnetite magnetosomes in these bacteria (Ding et al., J Bacteriol., 2010).

### B. Characterization of M. Magneticum ADAS Retention of Nanoparticles

Morphology of ADAS magnetosomes is analyzed using TEM as described by Wang et al., Front Microbiol., 2013. Briefly, 20 L of cells are dropped onto a copper TEM grid covered with carbon-coated formvar film for 2 h, then washed twice with sterilized distilled water and dried in air. Magnetosome sizes are defined as (length+width)/2, and the shape factors as width/length by measuring TEM micrographs.

### C. Demonstration of M. Magneticum ADAS Magnetism

To characterize the magnetic properties of M. magneticum ADAS, a protocol modified from Ding et al., J Bacteriol., 2010 is used. Briefly, samples are freeze-dried and low-temperature magnetic measurements are taken using a Quantum Design MPMS XL-5 magnetometer (sensitivity, 5.0×10-10 Am2). Thermal demagnetization of remanence acquired in a 2.5-T field at 5 K (hereafter named SIRM5K_2.5T) after two pretreatments is measured from 5 K to 300 K. The first pretreatment is cooling the sample from 300 K to 5 K in a zero field (zero field cooled [ZFC]), and the second is cooling it from 300 K to 5 K in a 2.5-T field (field cooled [FC]). The Verwey transition temperature (Tv) is defined as the temperature corresponding to the maximal first- order derivative dM/dT of the FC curve. Room temperature first- order reversal curves (FORCs) are measured on an alternating gradient magnetometer (sensitivity, 1.0×10-11 Am2; MicroMag model 2900). FORC diagrams are calculated using FORCinel version 1.05 software, with a smoothing factor (SF) of 2. FORC diagrams provide information on the domain state, coercivity, and magnetostatic interaction of magnetic crystals.

### Example 39. Environmentally Responsive ADAS

Logic operations based on Boolean logic gates can be encoded in gene regulatory networks to enable cells to integrate or differentiate between different environmental and cellular cues and respond accordingly. Customized genetic logic circuits can be designed to link various cellular sensors and actuators that are not found in native cells. These modified cells can be programmed to generate desired outcomes in response to specific inputs, intra- or extracellularly. Despite some drawbacks, such as the non-modularity of the gene circuits and limitations of the host chassis, they are extremely useful in engineered organisms and the library of genetic logic circuits is currently being expanded. A number of such circuits have been described in E. coli, which are incorporated into E. coli ADAS.

These circuits form the basis for environmentally responsive ADAS, which can produce a tunable output in response to the input (e.g. metabolites, pH, heat, light, external ligands). They are engineered with AND gates to respond to the presence of multiple inputs, or with OR gates to respond to any of the encoded inputs. This repertoire can be expanded to include other logic gates such as NOR, NAND, XOR etc.

a. Synthesis of E. Coli ADAS with AND/OR Gates

P. syringae contains a regulatory system for Type III secretion called the hrpR/hrpS system which forms an AND gate which tightly regulates the expression of T3SS. Expression of both these proteins is necessary for the production of the T3SS system. Plasmid construction and DNA manipulation are performed using standard molecular biology techniques, and the construction of the AND gate is modified from the protocol described by Wang et al., Nat Commun., 2011. Briefly, plasmids are constructed containing a) the IPTG-inducible Plac promoter and the hrpR portion of the AND gate, and b) the heat-induced promoter pL (from phage lambda which is usually suppressed by a thermolabile protein) and the hrpS portion of the AND gate. The output is GFP protein expression, which contains the T3SS promoter and is expressed when both hrpR and hrpR are expressed.

Plasmid construction and DNA manipulations are performed following standard molecular biology techniques. The hrpR and hrpS genes promoter are synthesized by GENEART following the BioBrick standard. Plasmid pAPT110 (p15A ori, Kanr) containing the IPTG-inducible Plac is used for characterization and harboring hrpR (Xbal/Kpnl) of the AND gate. Plasmid pBAD18-Cm (pBR322 ori, Cmr) containing the heat-inducible pL promoter harboring hrpS of the AND gate is obtained. pSB3K3 (p15A ori, Kanr) is used to clone and characterize the synthetic AHL-inducible Plux promoter (BBa_F2620) that is used later to drive hrpR (Xbal/Pstl). The various sequences for each gene construct are introduced by PCR amplification (using PfuTurbo DNA polymerase from Stratagene and an Eppendorf Mastercycler gradient thermal cycler) with primers containing the corresponding RBS and appropriate restriction sites. Alternatively, all constructs are synthesized commercially. All constructs are verified by DNA sequencing (Eurofins MWG Operon) before their use in target cell strains.

All characterization experiments are done in M9 minimal media (11.28 g M9 salts/l, 1 mM thiamine hydrochloride, 0.2% (w/v) casamino acids, 2 mM MgSO4, 0.1 mM CaCI2), supplemented with appropriate carbon source. Two M9 media with different carbon source are used: M9-glycerol (0.4% (v/V) glycerol) and M9-glucose (0.01% (v/v) glucose). The antibiotic concentrations used are 25 μg/ml for kanamycin, 25 ug ml-1 for chloramphenicol and 25 μg/ml for ampicillin. Cells inoculated from single colonies on freshly streaked plates are grown overnight in 4 ml M9 in 14 ml Falcon tubes at 37° C. with shaking (200 r.p.m.). Overnight cultures are diluted into pre-warmed M9 media at OD600=0.05 for the day cultures, which are induced and grown for 5 h at 30° C. before analysis, unless otherwise indicated. For fluorescence assay by fluorometry, diluted cultures are loaded into a 96-well microplate (Bio-Greiner, chimney black, flat clear bottom) and induced with 5 μL (for single-input induction) or 10 μL (for double-input induction) inducers of varying concentrations to a final volume of 200 μL per well by a multichannel pipette.

To engineer an OR gate, the system described by Rosado et al., PLOS Genetics, 2018 is used. First, a cis-repressed mRNA coding for RFP under a constitutive promoter is constructed. The repression is removed in the presence of RAJ11 sRNA. Plasmids are synthesized containing the IPTG-inducible promoter PLac and heat-induced promoter pL, both of which induce the expression of RAJ11 sRNA. The output is RFP expression, which is seen in response to either input.

Synthetic PL-based promoters regulated by the transcription factors Lacl and pl are used as elements to sense the input signals (IPTG and heat). Riboregulatory sequences (sRNAs and 5′ UTRs) of systems RAJ11 are obtained from previous work (Rodrigo et al., PNAS, 2012). The various sequences for each gene construct are introduced by PCR amplification (using PfuTurbo DNA polymerase from Stratagene and an Eppendorf Mastercycler gradient thermal cycler) with primers containing the corresponding RBS and appropriate restriction sites. All constructs are verified by DNA sequencing (Eurofins MWG Operon) before their use in target cell strains.

LB medium is used for overnight cultures, while M9 minimal medium (1× M9 salts, 2 mM MgSO4, 0.1 mM CaCI2, 0.4% glucose, 0.05% casamino acids, and 0.05% thiamine) for characterization cultures. Ampicillin and kanamycin are used as antibiotics at the concentration of 50 μg/mL.

GFP and RFP cultures are assayed in a fluorometer (Perkin Elmer Victor X5) to measure absorbance (600 nm absorbance filter), green fluorescence (485/14 nm excitation filter, 535/25 nm emission filter), and red fluorescence (570/8 nm excitation filter, 610/10 nm emission filter). Mean background values of absorbance and fluorescence, corresponding to M9 minimal medium, are subtracted to correct the readouts. Normalized fluorescence is calculated as the ratio of fluorescence and absorbance. The mean value of normalized fluorescence corresponding to cells transformed with control plasmids is then subtracted to obtain a final estimate of expression.

These gates are model systems and can be further modified to activate with any input, including other chemicals, cell contact, pH, and light.

B. Generation of ADAS with AND/OR Gates

ADAS are generated according to the protocol described in Example 12 and purified according to the methods in Example 13.

### C. Logic Gates are Functional in ADAS

As described previously, the hrpR/hrpS requires both inputs to generate product effectively creating an AND logic gate. E. coli ADAS are subjected to four conditions: 1. No IPTG/no heat, 2. ITPG/no heat, 3. No IPTG/heat, and 4. ITPG/heat. Only condition 4 where both heat and IPTG are present will cause expression of the hrpR/hrpS, which leads to expression of GFP.

GFP expression can potentially be modulated by changing the concentration of IPTG and duration of stimulus exposure. E. coli ADAS exposed to different conditions are loaded onto a 96-well plate, and a fluorescent plate reader is used to quantify GFP expression relative to different conditions. Double blind experiments confirm that presence of GFP indicates the presence of both IPTG and heat, forming the basis for a system that is responsive to stimuli.

### Example 40. Heavy Metal-Scavenging ADAS from E. Coli and Extremophiles

Heavy metals pose an environmental and health risk and are often difficult to remove in an efficient and noninvasive way. Bacteria can be engineered to scavenge heavy metals such as mercury, using the expression of proteins like MerR, which is a metalloregulatory protein with high affinity and selectivity towards mercury.

a. Synthesis of E. Coli Parent Lines Expressing MerR

E. coli parent lines are synthesized using methods described by Bae et al., App Environ Microbiol, 2003. Briefly, MerR is fused with an ice nucleation protein (INP) for surface expression. A hexahistidine tag is added to the C-terminus of the fusion protein to confirm expression.

Briefly, the INP-MerR fusion is constructed as follows. The merR fragment is PCR amplified from plasmid pT7 KB with the primers merR1 (5′ CCGGGATCCTATGGAAAACAATTTGGAGA 3′) and merR2 (5′ CAGCTGCAGCCCTAAGGCATAGCCGAACC 3′). The amplified fragment is digested with BamHI and Pstl, gel purified, and subcloned into a similarly digested pUNI, which contains an EcoRI-BamHI INP fragment inserted into pUC18Not, to generate pUNIM. The resulting construct allows expression of MerR on the surface of E. coli.

To probe the surface localization of MerR, a hexahistidine tag is added to the C-terminal part of the INP-MerR fusion. The merR fragment is reamplified with a new reverse primer, merR3 (5′ ATTCTGCAGCTAATGATGATGGTGGTGGTGATAAGGCATAGCCGAACCTGCCAAGCTT 3′), coding for six histidines at the C terminus. The resulting plasmid, pUNIMH, coding for the INP-MerR-H6 fusion, is prepared.

Mercury scavenging is measured by growing the bacterial clones along with untransformed E. coli as a control in LB broth in the presence of HgCI2 at concentrations of 0, 5, 10, 20, 40, 80, 100, 120, 140, and 160 M. The absorbance is measured at 600 nm for each bacterial clone after 16 and 120 hours of incubation in order to determine growth and their relative resistance to mercury.

### B. Synthesis and Purification of E. Coli ADAS

ADAS are generated according to the protocol described in Example 12 and purified according to the methods in Example 13.

### C. Mercury Scavenging by E. Coli ADAS

E. coli ADAS are incubated for 16 and 120 hours in LB broth containing HgCI2 at concentrations of 0, 5, 10, 20, 40, 80, 100, 120, 140, and 160 μM. Residual levels of mercury are tested using assays designed for detecting mercury to extremely low levels (e.g. chelating strip designed by Brummer et al., Bioorg. Med. Chem., 2001)

### Example 41. Lactose Uptake and Metabolism by E. Coli ADAS

Lactose uptake in E. coli occurs via the membrane-bound protein beta-galactoside permease, which enables transport of lactose and other beta-galactosides into the cell. Transport of the sugar molecule is accompanied by cotransport of a proton, which allows assaying of uptake using pH sensitive dyes.

a. Synthesis of E. Coli Parent Lines Expressing Beta-Galactosidase Permease

E. coli parent lines are synthesized using a broth containing only lactose as a sugar source. E. coli contain the lac operon system for uptake and utilization of lactose as a source of sugar in the absence of preferred sugars. The removal of preferred sugars induces expression of the proteins involved in the uptake and metabolism of lactose. Since uptake is via a membrane-bound transporter, induction of this system is necessary to increase transporter expression in the parent line, which partly determines the expression in ADAS. Additionally, plasmids containing the lac operon system are introduced into E. coli ADAS for expression of the transporter.

### B. Lactose Uptake by E. Coli ADAS

E. coli ADAS are synthesized and purified as described in Example 12 and 13. Lactose uptake by E. coli ADAS is measured using a pH sensitive assay. As described by Prabhala et al., FEBS Letters, 2014, the pH sensitive dye pyranine is used to measure lactose uptake. Briefly, E. coli ADAS are pelleted and washed at least thrice with unbuffered Krebs solution containing 140 mM NaCl, 5.4 mM KCl, 1.8 mM CaCI2, 0.8 mM MgSO4, 0.3 mM pyranine and varying concentrations of lactose (1 mM-5 mM), and resuspended. The pH of the suspension is carefully adjusted to 6.5 while nitrogen is bubbled through the suspension for 5 min and 30 mL liquid paraffin is added above the cell suspension to avoid changes in pH as a result of carbon dioxide dissolution. The cell suspension is transferred directly to the assay plates for fluorescence measurements (using a fluorimeter at an excitation wavelength of 455 nm and emission wavelength of 509 nm). Control experiments are performed using empty transformed E. coli cells as negative controls, and parent lines as positive controls.

C. E. Coli ADAS that Convert Lactose into Glucose and Galactose

In the absence of glucose, E. coli use the bacterial operon lac operon to use lactose as a sugar source. Bacterial operons are polycistronic transcripts that are able to produce multiple proteins from one mRNA transcript, which are lacZ, lacY and lacA in the case of the lac operon. The gene products of lacZ, lacY and lacA are beta-galactosidase, which cleaves lactose into glucose and galactose, beta-galactoside permease, which enables transport of lactose into the cell, and galactoside acetyltransferase, which is an enzyme that transfers an acetyl group from acetyl-CoA to galactosides, glucosides and lactosides respectively. Conversion of lactose into glucose and galactose is mediated via the enzyme beta-galactosidase, the function of this enzyme can be directly measured using an enzyme-activity assays.

### D. Synthesis and Purification of E. Coli ADAS

E. coli ADAS are synthesized and purified as described in Example 12 and 13. Lactose uptake is evaluated in E. Coli ADAS in the absence of glucose.

E. Conversion of Lactose into Glucose and Galactose by E. Coli ADAS

To evaluate the activity of the enzyme beta-galactoside permease, E. coli ADAS are collected and first lysed using a lysis buffer with protease inhibitors to prevent protein degradation, for example, as described by EMBL protocol.

The activity of this enzyme is measured using a commercially available kit from ThermoFisher (Catalogue #K145501) using the manufacturer's instructions. Briefly, beta-galactosidase catalyzes the hydrolysis of β-galactosides such as ortho-nitrophenyl-D-galactopyranoside (ONPG). Hydrolysis of ONPG to the ONP anion produces a bright yellow color. The β-galactosidase activity of the solution is quantitated using a spectrophotometer or a microplate reader to determine the amount of substrate converted at 420 nm.

E. coli parent lines are used as a positive control. Ability of E. coli ADAS to break down lactose will be determined by the lactose conversion index (L), where L=(enzyme activity in E. coli ADAS)/(enzyme activity in parent line)×100

### Example 42. PETase-Expressing E. Coli ADAS for Degradation of Plastic

Ideonella sakaiensis is a bacterium isolated from outside a bottle recycling facility that can break down and metabolize a common plastic, poly-(ethylene teraphthalate) or PET (Yoshida et al., Science, 2016). It secretes the enzyme PETase to break down the polymer into monomer. E. Coli ADAS expressing I. sakaiensis PETase are capable of degrading the widely used plastic PET (˜3500 million pounds of PET bottles alone end up as landfill in the US annually).

a. Transformation of E. Coli to Express PETase

PETase-synthesizing E. coli ADAS parent lines are synthesized using the protocol described by Han et al., Nat. Commun., 2017. Briefly, the PETase from Ideonella sakaiensis (GenBank accession number: GAP38373.1) without the N-terminal 29 amino acids is cloned and ligated into the pET32a vector commercially. The pET32a-PETase plasmid, either wild type or variants, is transformed into E. coli BL21trxB (DE3) cells that are grown in LB medium at 37° C. to an OD600 of ˜0.8 and then induced by 0.6 mM isopropyl β-d-thiogalactopyranoside (IPTG) at 16° C. for 24 h. Parent lines are incubated with PET films (described below in c) to confirm PETase activity.

### B. Synthesis and Purification of E. Coli ADAS

E. coli ADAS are synthesized and purified as described in Example 12 and 13. Lactose uptake is evaluated in E. Coli ADAS in the absence of glucose.

### C. Using PETase Expressing of E. Coli ADAS to Degrade PET

Ideonella sakaiensis 201-F6 is obtained from BCRC and cultured according to manufacturer's instructions. This serves as a positive control to measure the activity of PETase-expressing E. coli.

Ability to degrade PET is measured using the protocol described by Yoshida et al., Science, 2016. Briefly, cultured samples of I. sakaiensis and E. coli ADAS are suspended in 10 mM phosphate buffer (pH 7.0) with low crystallinity PET thin film (PET film) (ca. 60 mg, 20×15×0.2 mm, Mw: 45×103, Mw/Mn: 1.9, Tg: 77° C., Tm: 255° C., crystallinity: 1.9%, density: 1.3378 g/cm³). The PET film is sterilized in 70% ethanol and dried in sterile air before being placed in the test tube. The tube is shaken at 300 strokes/min at 30° C.

Quantification of CO₂ generation is used as a measure of PET breakdown. The generated CO₂ is entrapped in Ascarite and the weight is measured to calculate the absorbed CO₂. The biofilm-like materials and treated PET films are separated from the medium to determine the carbon weight of the degraded film. The conversion rate from PET to CO₂ (R) is calculated as follows.

$$R \% = CO 2 ( PET + ) \times CO 2 ( PET - ) \times 100$$

Carbon weight of degraded film
where CO₂ (PET+) and CO₂ (PET−) indicate the carbon weight of generated CO₂ cultivated in the presence and absence of PET, respectively.
Plastic degradation efficiency of E. coli ADAS is measured using the PETase index (Y), which is calculated as follows:

$$Y = R ( E . coli ADAS ) / R ( I . sakaiensis )$$

where R indicates the conversion rate shown above.
ADAS can be further optimized to express mutated PETase enzymes that have been shown to have greater activity and to increase the range of plastics degraded, as described by Austin et al., PNAS, 2018.

### Example 43. RNA Secretion Via a T4 Secretion System

It has been shown that T4SS are capable of secreting DNA, but it has yet to be shown that T4SS are capable of secreting RNA. This example describes secretion of RNA via the coupling of an RNA-binding protein to the RNA.

a. Synthesis of Agrobacterium with RNA-Secreting T4 Secretion System

Cas9 optimized for S. cerevisiae (from Generoso et al, J. Microbiol. Meth., 2016) and tombusvirus p19 (Uniprot Q66104) sequences are fused to the N-terminal of VirE2 and VirF under the virF promoter. An sgRNA or siRNA sequence is also put on this sequence under control of the virF promoter. The sgRNA sequence against ILV1 is described in Generoso et al, J. Microbiol. Meth., 2016. The protocol is as described in Vergunst et al, PNAS, 2005 and Vergunst et al, Science, 2000 with some modifications. Briefly, the plasmids are synthesized commercially and electroporated into Agrobacterium containing T4SS systems and agrobacterium without T4SS systems. The agrobacterium is then cultured on LB plates and then grown up according to standard protocols. The presence of the fusion protein is verified via western blot. Furthermore, the sgRNA or siRNA sequence is verified via Quantigene assay after RNA isolation with commercial small RNA isolation kits from Thermo Fisher.

### B. Demonstrating T4SS Delivery of RNA to Yeast

The protocol for delivery of T4SS to yeast is described in Schrammeijer et al., Nucl. Acids Res., 2003. Briefly the agrobacterium strains are grown overnight in minimal medium with antibiotic, harvested and diluted in induction medium, and then grown for 5 h before usage. S. cerevisiae strains are grown overnight in standard medium, diluted 1:10, and regrown for another 5 h. Cultures of the agrobacterium and the yeast are mixed together 1:1 and grown on cellulose nitrate filters. After 6 days of co cultivation, the mixture is analyzed by plating onto yeast medium with cefotaxim and yeast colonies are grown and analyzed by Quantigene assays for presence of the delivered RNA after RNA isolation with commercial small RNA isolation kits from Thermo Fisher.

### C. Demonstrating T4SS Mediated Editing of DNA in Yeast

Following the protocol mentioned above, yeast colonies are grown in media without isoleucine and colonies are measured for those cocultured with Agrobacterium with and without T4SS and with and without the sgRNA/Cas9 cassette.

### Other Embodiments

Some embodiments of the invention are within the following numbered paragraphs.

1. An isolated highly active achromosomal dynamic active system (ADAS).

2. The highly active ADAS of paragraph 1, comprising an ATP synthase concentration of at least: 1 per 10000 nm², 1 per 5000 nm², 1 per 3500 nm², 1 per 1000 nm².

3. The highly active ADAS of any one of the preceding paragraphs, wherein the ADAS comprises an ATP synthase, optionally lacking a regulatory domain, such as lacking an epsilon domain.

4. The highly active ADAS of any one of the preceding paragraphs, further comprising a photovoltaic proton pump.

5. The highly active ADAS of paragraph 4, wherein the photovoltaic proton pump is a proteorhodopsin.

6. The highly active ADAS of paragraph 5, wherein the proteorhodopsin comprises the amino acid sequence of proteorhodopsin from the uncultured marine bacterial clade SAR86, GenBank Accession: AAS73014.1.

7. The highly active ADAS of paragraph 4, wherein the photovoltaic proton pump is a gloeobacter rhodopsin.

8. The highly active ADAS of paragraph 4, wherein the photovoltaic proton pump is a bacteriorhodopsin, deltarhodopsin, or halorhodopsin from Halobium salinarum Natronomonas pharaonis, Exiguobacterium sibiricum, Haloterrigena turkmenica, or Haloarcula marismortui.

9. The highly active ADAS of any one of the preceding paragraphs, further comprising retinal.

10. The highly active ADAS of any one of the preceding paragraphs, further comprising a retinal synthesizing protein (or protein system), or a nucleic acid encoding the same.

11. The highly active ADAS of any one of the preceding paragraphss further comprising one or more glycolysis pathway proteins.

12. The highly active ADAS of paragraph 11 wherein the glycolysis pathway protein is a phosphofructokinase (Pfk-A).

13. The highly active ADAS of paragraph 12, wherein the Pfk-A comprises the amino acid sequence of UniProt accession POA796.

14. The highly active ADAS of paragraph 12, wherein the glycolysis pathway protein is triosephosphate isomerase (tpi).

15. The highly active ADAS of paragraph 14, wherein the tpi comprises the amino acid sequence of UniProt accession POA858.

16. The highly active ADAS of any one of the preceding paragraphs, which lacks one or more metabolically non-essential proteins.

17. The highly active ADAS of any one of the preceding paragraphs, which lacks one or more of an RNAse, a protease, or a combination thereof, and, in particular embodiments, lacks one or more endoribosucleases (such as RNAse A, RNAse h, RNAse III, RNAse L, RNAse PhyM) or exoribonucleases (such as RNAse R, RNAse PH, RNAse D); or serine, cysteine, threonine, aspartic, glutamic and metallo-proteases; or a combination of any of the foregoing.

18. The highly active ADAS of any one of the preceding paragraphs further comprising a bacterial secretion system.

19. The highly active ADAS of paragraph 18, wherein the bacterial secretion system is capable of exporting a cargo across the ADAS outer membrane into a target cell, such as an animal, fungal, bacterial, or plant cell.

20. The highly active ADAS of paragraph 18 or 19, wherein the bacterial secretion system is T3SS, T4SS, or T6SS.

21. The highly active ADAS of paragraph 18 or 19, wherein the bacterial secretion system is a T3/4SS.

22. The highly active ADAS of paragraph 21, wherein the T3/4SS has modified effector function, e.g., an effector selected from SopD2, SopE, Bop, Map, Tir, EspB, EspF, NleC, NleH2, or NleE2.

23. The highly active ADAS of paragraph 22, wherein the modified effector function is for intracellular targeting such as translocation into the nucleus, golgi, mitochondria, actin, microvilli, ZO-1, microtubules, or cytoplasm.

24. The highly active ADAS of paragraph 22, wherein modified effector function is nuclear targeting based on NleE2 derived from E. Coli.

25. The highly active ADAS of paragraph 22, wherein the modified effector function is for filopodia formation, tight junction disruption, microvilli effacement, or SGLT-1 inactivation.

26. The highly active ADAS of paragraph 18 or 19, wherein the bacterial secretion system is a T6SS.

27. The highly active ADAS of paragraph 26, wherein the T6SS, in its natural host, targets a bacterium and contains an effector that kills the bacteria.

28. The highly active ADAS of paragraph 26 or paragraph 27, wherein the T6SS is derived from P. putida K1-T6SS and, optionally, wherein the effector comprises the amino acid sequence of Tke2 (Accession AUZ59427.1).

29. The highly active ADAS of paragraph 26, wherein the T6SS, in its natural host, targets a fungi and contains an effector that kills fungi.

30. The highly active ADAS of paragraph 29, wherein the T6SS is derived from Serratia Marcescens and the effectors comprise the amino acid sequences of: Tfe1 (Genbank: SMDB11_RS05530) or Tfe2 (Genbank: SMDB11_RS05390).

31. The highly active ADAS of paragraph 18, wherein the bacterial secretion system is capable of exporting a cargo extracellularly.

32. The highly active ADAS of paragraph 31, wherein the bacterial secretion system is T1SS, T2SS, T5SS, T7SS, Sec, or Tat.

33. The highly active ADAS of any one of the preceding paragraphs, comprising a transporter in the membrane.

34. The highly active ADAS of paragraph 33, where the transporter is specific for glucose, sodium, potassium, a metal ion, an anionic solute, a cationic solute, or water.

35. The highly active ADAS of any one of the preceding paragraphs, wherein the membrane comprises a targeting agent.

36. The highly active ADAS of paragraph 35, wherein the targeting agent is a nanobody, such as a nanobody directed to a tumor antigen, such as HER2, PSMA, or VEGF-R.

37. The highly active ADAS of paragraph 35, where the targeting agent is a carbohydrate binding protein, such as a lectin, e.g. Mannose Binding Lectin (MBL).

38. The highly active ADAS of paragraph 35, where the targeting agent is a tumor-targeting peptide, such as an RGD motifs or CendR peptide.

39. The highly active ADAS of any one of the preceding paragraph 5, wherein the ADAS membrane comprises an enzyme.

40. The highly active ADAS of paragraph 39, wherein the enzyme is a protease, oxidoreductase, or a combination thereof.

41. The highly active ADAS of paragraph 39 or paragraph 40, wherein the enzyme is chemically conjugated to the ADAS membrane, optionally via a linker to the exterior membrane.

42. The highly active ADAS of any one of the preceding paragraph 5, comprising a cargo dispersed in the interior volume of the ADAS, wherein the cargo comprises a nucleic acid, ribosome, peptide, hormone, amino acid, carbohydrate, lipid, protein, organic particle, inorganic particle, small molecule, or a combination thereof.

43. The highly active ADAS of any one of the preceding paragraph 5, wherein the ADAS comprises a bacterial secretion system and a cargo, optionally wherein the cargo comprises a moiety that directs export by the bacterial secretion system, e.g., in some embodiments the moiety is Pho/D, Tat, or a synthetic peptide signal.

44. The ADAS of paragraph 42 or 43, wherein the cargo is modified for improved stability compared to an unmodified version of the cargo.

45. The highly active ADAS of any one of paragraphs 42-44, wherein the cargo comprises a protein.

46. The highly active ADAS of paragraph 45, wherein the protein is a hormone, e.g., paracrine, endocrine, autocrine.

47. The highly active ADAS of paragraph 60 or paragraph 61, wherein the protein has stability greater than about: 1.01, 1.1, 10, 100, 1000, 10000, 100000, 100000, 10000000 in cell cytoplasm or other environments.

48. The highly active ADAS of any one of paragraphs 42-44, wherein the cargo comprises a plant hormone, such as abscisic acid, auxin, cytokinin, ethylene, gibberellin, or a combination thereof.

49. The highly active ADAS of any one of paragraphs 42-44, wherein the cargo is an immune modulator, such as an immune stimulator, check point inhibitors (e.g., of PD-1, PD-L1, CTLA-4), suppressors, super antigens, small molecule (cyclosporine A, cyclic dinucleotides (CDNs), or STING agonist (e.g., MK-1454)).

50. The highly active ADAS of any one of paragraphs 42-44, wherein the cargo comprises an RNA, such as circular RNA, mRNA, siRNA, shRNA, ASO, tRNA or a combination thereof.

51. The highly active ADAS of paragraph 50, wherein the cargo comprises a protein-coding mRNA.

52. The highly active ADAS of paragraph 51, wherein the protein-coding mRNA encodes an enzyme (e.g., and enzyme that imparts hepatic enzymatic activity, such as human PBGD (hPBGD) mRNA) or an antigen, e.g., that elicits an immune response (such as eliciting a potent and durable neutralizing antibody titer), such as mRNA encoding CMV glycoproteins gB and/or pentameric complex (PC)).

53. The highly active ADAS of paragraph 50, wherein the RNA is a small non-coding RNA, such as shRNA,

ASO, tRNA or a combination thereof.

54. The highly active ADAS of any one of paragraphs 50-53, wherein the RNA is stabilized, e.g., with an appended step-loop structure, such as a tRNA scaffold.

55. The ADAS of any one of paragraphs 65-70, wherein the RNA has stability greater than about: 1.01, 1.1, 10, 100, 1000, 10000, 100000, 100000, 10000000, e.g., in ADAS plasm.

56. The highly active ASAS of any one of the preceding paragraphs, wherein the highly active ADAS does not degrade a cargo.

57. The highly active ADAS of paragraph 42 or 43, wherein the cargo comprises a gene editing system.

58. The highly active ADAS of paragraph 58, wherein cargo is DNA, such as a plasmid, or a circular RNA, optionally wherein the DNA or circular RNA comprises a protein-coding sequence.

59. The highly active ADAS of any one of the preceding paragraphs, wherein the ADAS is a two-membrane ADAS.

60. The highly active ADAS of paragraph 59, wherein the two-membrane ADAS further comprises a bacterial secretion system.

61. The highly active ADAS of paragraph 60, wherein the bacterial secretion system is selected from T3SS, T4SS, T3/4SS, or T6SS, optionally wherein the T3SS, T4SS, T3/4SS, or T6SS has an attenuated or non-functional effector that does not affect fitness of a target cell.

62. The highly active ADAS of any one of paragraphs 59-61, which is derived from a bacterial parental strain, wherein the parental strain is selected from a plant bacterium, such as a plant commensal (e.g., B. Subtilis or Pseudomonas putida) or a plant pathogen bacterium (e.g., Xanthomonas sp. Or Psuedomonas syringae) or a human bacterium, such as a commensal human bacterium (e.g., E. coli, Staphylococcus sp., Bifidobacterium sp., Micrococcus sp., Lactobacillus sp., or Actinomyces sp.) or a pathogenic human bacterium (e.g., Escherichia coli EHEC, Salmonella Typhimurium, Shigella flexneri, Yersinia enterolitica, Helicobacter pylori), or an extremophile, including functionalized derivatives of any of the foregoing, for example including a functional cassette, such as a functional cassette that induces the bacterium to do one or more of: secrete antimicrobials, digest plastic, secrete insecticides, survives extreme environments, make nanoparticles, integrate within other organisms, respond to the environment, and create reporter signals.

63. The highly active ADAS of any one of the preceding paragraphs, which is derived from a parental strain selected from E. coli, Agrobacterium, Rhizobium, Pseudomonas, Xanthomonas, Anaplasma, Helicobacter, Serratia, Vibrio, Salmonella, or Shigella.

64. The highly active ADAS of any one of the preceding paragraphs, which is derived from a parental strain engineered or induced to overexpress ATP synthase.

65. The highly active ADAS of paragraph 64, wherein the ADAS comprises an ATP synthase heterologous to the parental strain.

66. The highly active ADAS of paragraph 64 or 65, wherein the parental strain is modified to express a functional FoF1 ATP synthase.

67. The highly active ADAS of any one of the preceding paragraphs, which is obtained from a parental strain cultured under a condition selected from: applied voltage (e.g., 37 mV), non-atmospheric oxygen concentration (e.g., 1-5% 02, 5-10% 02, 10-15% 02, 25-30% 02), low pH (about: 4.5, 5.0, 5.5, 6.0, 6.5), or a combination thereof.

68. The highly active ADAS of any one of the preceding paragraphs, which is derived from a parental strain auxotrophic for at least 1, 2, 3, 4, or more of: arginine (e.g., knockout in argA, such as strains JW2786-1 and NK5992), cysteine knockout in cysE (such as strains JW3582-2 and JM15), glutamine e.g., knockout in glnA (such as strains JW3841-1 and M5004), glycine e.g., knockout in glyA (such as strains JW2535-1 and AT2457), Histidine e.g., knockout in hisB (such as strains JW2004-1 and SB3930), isoleucine e.g., knockout in ilvA (such as strains JW3745-2 and AB1255), leucine e.g., knockout in leuB (such as strains JW5807-2 and CV514), lysine e.g., knockout in lysA (such as strains JW2806-1 and KL334), methionine e.g., knockout in metA (such as strains JW3973-1 and DL41), phenylalanine e.g., knockout in pheA (such as strains JW2580-1 and KA197), proline e.g., knockout in proA (such as strains JW0233-2 and NK5525), Serine e.g., knockout in serA (such as strains JW2880-1 and JC158), threonine e.g., knockout in thrC (such as strains JW0003-2 and Gif 41), tryptophan e.g., knockout in trpC (such as strains JW1254-2 and CAG18455), Tyrosine e.g., knockout in tyrA (such as strains JW2581-1 and N3087), Valine/Isoleucine/Leucine e.g., knockout in ilvd (such as strains JW5605-1 and CAG18431).

69. The highly active ADAS of any one of the preceding paragraphs, which is made from a bacterial cell, wherein the parental strain is selected from a plant bacterium, such as a plant commensal (e.g., B. Subtilis or Pseudomonas putida) or a plant pathogen bacterium (e.g., Xanthomonas sp. Or Psuedomonas syringae) or a human bacterium, such as a commensal human bacterium (e.g., E. coli, Staphylococcus sp., Bifidobacterium sp., Micrococcus sp., Lactobacillus sp., or Actinomyces sp.) or a pathogenic human bacterium (e.g., Escherichia coli EHEC, Salmonella Typhimurium, Shigella flexneri, Yersinia enterolitica, Helicobacter pylori), or an extremophile, including functionalized derivatives of any of the foregoing, for example including a functional cassettes, such as a functional cassette that induces the bacterium to do one or more of: secrete antimicrobials, digest plastic, secrete insecticides, survives extreme environments, make nanoparticles, integrate within other organisms, respond to the environment, and create reporter signals.

70. The highly active ADAS of any of the preceding paragraphs comprising a modified membrane.

71. The highly active ADAS of paragraph 70, where the membrane is modified to be less immunogenic or immunostimulatory in plants or animals.

72. The highly active ADAS of paragraph 71, which is made from a parental strain, wherein the immunostimulatory capabilities of the parental strain are reduced or eliminated through post production treatment with detergents, enzymes, or functionalized with PEG.

73. The highly active ADAS of paragraph 71, which is made from a parental strain and the membrane is modified through knockout of LPS synthesis pathways in the parental strain, e.g., by knocking out msbB and/or purl.

74. The highly active ADAS of paragraph 71, which is made from a parental strain that produces cell wall-deficient particles through exposure to hyperosmotic conditions.

75. A composition comprising the highly active ADAS of any one of the preceding paragraphs.

76. A composition comprising ADAS wherein at least about: 80, 81, 82, 83, 84, 85, 90, 95, 96, 97, 98, 99, 99.1, 99.2, 99.3, 99.4, 99.5, 99.6, 99.7, 99.8, 99.9%, or more of the ADAS contain a bacterial secretion system.

77. The composition of paragraph 76, wherein the bacterial secretion system is one of T3SS, T4SS, T3/4SS, or T6SS.

78. A composition of ADAS, comprising a T3SS, wherein the ADAS comprise a mean T3SS membrane density greater than 1 in about: 40000, 35000,30000, 25000, 19600, 15000, 10000, or 5000 nm².

79. The composition of paragraph 78, wherein the ADAS is derived from a S. Typhimurium or E. coli parental strain.

80. A composition of ADAS comprising a T3SS, wherein the ADAS comprise a mean T3SS membrane density greater than 1 in about: 300000, 250000, 200000, 150000, 100000, 50000, 20000, 10000, 5000 nm².

81. The composition of paragraph 80, where the ADAS is derived from an Agrobacterium tumefaciens parental strain.

82. A composition of ADAS, wherein at least about: 80, 81, 82, 83, 84, 85, 90, 95, 96, 97, 98, 99, 99.1, 99.2, 99.3, 99.4, 99.5, 99.6, 99.7, 99.8, 99.9%, or more of the ADAS contain a bacterial secretion system, including T3, T4, T3/4SS, T6SS, and optionally including one or more of: exogenous carbohydrates, phosphate producing synthases, light responsive proteins, import proteins, enzymes, functional cargo, organism-specific effectors, fusion proteins.

83. The composition of any one of paragraphs 75-82, wherein the ADAS are highly active ADAS.

84. The composition of any one of paragraphs 75-83, which is formulated for IP, IV, IM, oral, topical (cream, gel, ointment, transdermal patch), aerosolized, or nebulized administration.

85. The composition of any one of paragraphs 75-84, which is a liquid formulation, or a lyophilized formulation.

86. A method of making any one of the ADAS of paragraphs 1-74, comprising culturing a parental stain under conditions to promote ADAS formation, and collecting the ADAS, optionally further comprising a step of isolating the ADAS from any residual parental strain cells or other contaminants.

87. The method of paragraph 86, comprising using a single, double, triple, or quadruple auxotrophic parental strain, said parental strain further comprising a plasmid expressing a ftsZ.

88. The method of paragraph 86, comprising transforming a parental strain with an inducible DNAse system, such as the exol (NCBI GeneID: 946529) & sbcD (NCBI GenelD: 945049) nucleases, or the I-Ceul (e.g., Swissprot: P32761.1) nuclease.

89. The method of paragraph 88, comprising using a single, double, triple, or quadruple auxotrophic strain and having the complementary genes on the plasmid encoding the inducible nucleases.

90. The method of paragraph 86, wherein the parental strain is cultured under a condition selected from: applied voltage (e.g., 37 mV), non-atmospheric oxygen concentration (e.g., 1-5% 02, 5-10% 02, 10-15% 02, 25-30% O₂), low pH (4.5-6.5), or a combination thereof. 91. The method of paragraph 86, wherein the parental strain lacks flagella and undesired secretion systems, optionally wherein the flagella and undesired secretion systems are removed using lambda red recombineering. 92. The method of paragraph 86, where flagella control components are excised from the parental strain genome via the insertion of a plasmid containing a CRISPR domain that is targeted towards flagella control genes, such as flhD and flhC.

93. A method of making a highly active ADAS, wherein an ADAS comprising a plasmid containing a rhodopsin-encoding gene is cultured in the presence of light.

94. The method of paragraph 93, where the rhodopsin is proteorhodopsin from SAR86 uncultured bacteria, having the amino acid sequence of GenBank Accession: AAS73014.1, and further optionally the culture is supplemented with retinal.

95. The method of paragraph 93 or 94, where the rhodopsin is proteorhodopsin and the plasmid additionally contains genes synthesizing retinal (such a plasmid is the pACYC-RDS plasmid from Kim et al., Microb Cell Fact, 2012).

96. The method of any one of paragraphs 86-95, wherein the parental strain contains a nucleic acid sequence encoding a nanobody that is then expressed on the membrane of the ADAS.

97. The method of any one of paragraphs 86-95, wherein the parental strain contains a nucleic acid sequence encoding one or more bacterial secretion system operons.

98. The method of any one of paragraphs 86-97, wherein parental strain comprises a cargo.

99. The method of any one of paragraphs 86-97, wherein the parental strain contains a nucleic acid sequence encoding a set of genes that synthesize a small molecule cargo.

100. An ADAS made by the method of any one of paragraphs 86-99.

101. A method of modulating a state of an animal cell, comprising providing an effective amount of an ADAS or composition of any one of the preceding paragraphs access to the animal cell.

102. The method of paragraph 101, wherein the ADAS or composition is provided access to the animal cell in vivo, in an animal, such as a mammal, such as a human.

103. The method of paragraph 102, wherein the animal cell is exposed to bacteria in a healthy animal.

104. The method of paragraph 103, wherein the animal cell is lung epithelium, an immune cell, skin cell, oral epithelial cell, gut epithelial cell, reproductive tract epithelial cell, or urinary tract cell.

105. The method of paragraph 104, wherein the animal cell is a gut epithelial cell, such as a gut epithelial cell from a human subject with an inflammatory bowel disease, such as Crohn's disease or colitis.

106. The method of paragraph 105, wherein the animal cell is a gut epithelial cell from a subject with an inflammatory bowel disease, and the ADAS comprises a bacterial secretion system and a cargo comprising an anti-inflammatory agent.

107. The method of paragraph 102, wherein the animal cell is exposed to bacteria in a diseased state.

108. The method of paragraph 102, wherein the animal cell is pathogenic, such as a tumor.

109. The method of paragraph 102, wherein the animal cell is exposed to bacteria in a diseased state, such as a wound, an ulcer, a tumor, or an inflammatory disorder

110. The method of any one of paragraphs 102-109, wherein the ADAS is derived from an animal commensal parental strain.

111. The method of any one of paragraphs 102-109, wherein the ADAS is derived from animal pathogenic parental strain.

112. The method of any one of paragraph 102, wherein the animal cell is contacted to an effective amount of an ADAS comprising a T3/4SS or T6SS and a cargo, wherein the cargo is delivered into the animal cell.

113. The method of paragraph 102, wherein the animal cell is provided access to an effective amount of an ADAS comprising a cargo and a secretion system, wherein the cargo is secreted extracellularly and contacts the animal cell.

114. A method of modulating a state of an animal cell, comprising providing an effective amount of an ADAS or composition of any one of paragraphs 1-85 access to a bacterial or fungal cell in the vicinity of the animal cell.

115. The method of paragraph 114, wherein the bacterial or fungal cell is pathogenic.

116. The method of paragraph 115, wherein the fitness of the pathogenic bacterial or fungal cell is reduced.

117. The method of paragraph 114, wherein the bacterial or fungal cell is commensal.

118. The method of paragraph 117, wherein the fitness of the commensal bacterial or fungal cell is increased.

119. The method of paragraph 118, wherein the fitness is increased via reduction in fitness of number of a competing bacteria or fungi, which may be neutral, commensal, or pathogenic.

120. The method of paragraph 114, wherein the bacterial or fungal cell is contacted to an effective amount of ADAS comprising a T3/4SS or T6SS and a cargo, wherein the cargo is delivered into the bacterial or fungal cell.

121. The method of claim 120, wherein the bacterial or fungal cell is provided access to an effective amount of ADAS secreting cargo extracellularly that contacts the bacterial or fungal cell.

122. The method of any one of paragraphs 114-121, wherein the ADAS is derived from a parental strain that is a competitor of the bacterial or fungal cell.

123. The method of any one of paragraphs 114-121, wherein the ADAS is derived from a from a parental strain that is mutualistic bacteria of the bacterial or fungal cell.

124. A method of modulating a state of a plant or fungal cell, comprising providing an effective amount of an ADAS or composition of any one paragraphs 1-85 access to: a) the plant or fungal cell, b) an adjacent bacterial or adjacent fungal cell in the vicinity of the plant or fungal cell, or c) an insect or nematode cell in the vicinity of the plant or fungal cell.

125. The method of paragraph 124, wherein the ADAS or composition is provided access to the plant cell in planta, e.g., in a crop plant such as row crops, including corn, wheat, soybean, and rice, and vegetable crops including solanaceae, such as tomatoes and peppers; cucurbits, such as melons and cucumbers; brassicas, such as cabbages and broccoli; leafy greens, such as kale and lettuce; roots and tubers, such as potatoes and carrots; large seeded vegetables, such as beans and corn; and mushrooms.

126. The method of paragraph 125, wherein the plant or fungal cell is exposed to bacteria in a healthy plant or fungus.

127. The method of paragraph 125, wherein the plant or fungal cell is exposed to bacteria in a diseased state.

128. The method of paragraph 125, wherein the plant or fungal cell is dividing, such as a meristem cell, or is pathogenic, such as a tumor.

129. The method of paragraph 125, wherein the plant or fungal cell is exposed to bacteria in a diseased state, such as a wound, or wherein the plant or fungal cell is not part of a human foodstuff.

130. The method of any one of paragraphs 124-129, wherein the ADAS is derived from a commensal parental strain.

131. The method of any one of paragraphs 124-129, wherein the ADAS is derived from a plant or fungal pathogenic parental strain.

132. The method of paragraph 124, wherein the ADAS comprises an T3/4SS or T6SS and a cargo, and the cargo is delivered into the plant or fungal cell.

133. The method of claim 124, wherein the plant or fungal cell is provided access to an effective amount of an ADAS comprising a bacterial secretion system and a cargo, wherein the bacterial secretion system secretes the cargo extracellularly, thereby contacting the plant or fungal cell with the cargo.

134. The method of paragraph 124, comprising providing an effective amount of an ADAS or composition access to the adjacent bacterial or adjacent fungal cell in the vicinity of the plant or fungal cell.

135. The method of claim 134, wherein the adjacent bacterial or adjacent fungal cell is pathogenic, optionally wherein the fitness of the pathogenic adjacent bacterial or adjacent fungal cell is reduced.

136. The method of claim 134, wherein the adjacent bacterial or adjacent fungal cell is commensal, optionally wherein the fitness of the commensal adjacent bacterial or adjacent fungal cell is increased.

137. The method of paragraph 136, wherein the fitness is increased via reduction of a competing bacteria or competing fungi, which may be neutral, commensal, or pathogenic.

138. The method of paragraph 134, wherein the adjacent bacterial or adjacent fungal cell is contacted with an effective amount of ADAS comprising a T3/4SS or T6SS and a cargo, wherein the cargo is delivered into the adjacent bacterial or adjacent fungal cell.

139. The method of paragraph 134, wherein the adjacent bacterial or adjacent fungal cell is provided access to an effective amount of ADAS comprising a bacterial secretion system and a cargo, wherein the bacterial secretion system secretes the cargo extracellularly, thereby contacting the adjacent bacterial or adjacent fungal cell with the cargo.

140. The method of any one of paragraphs 134-139, wherein the ADAS is derived from a parental strain that is a competitor of the adjacent bacterial or adjacent fungal cells.

141. The method of any one of paragraphs 134-139, wherein the ADAS is derived from a parental strain that is a mutualistic bacteria of the adjacent bacterial or adjacent fungal cell.

142. The method of paragraph 124, comprising providing an effective amount of the ADAS or composition access to an insect or nematode cell in the vicinity of the plant or fungus.

143. The method of paragraph 142, wherein the insect or nematode is pathogenic.

144. The method of paragraph 142, wherein the fitness of the pathogenic insect or nematode cell is reduced.

145. The method of paragraph 144, wherein the fitness of the pathogenic insect or nematode cell is reduced via modulation of symbiotes in the insect or nematode cell.

146. The method of paragraph 142, wherein the insect or nematode is commensal.

147. The method of paragraph 146, wherein the fitness of the commensal insect or nematode cell is increased.

148. The method of paragraph 147, wherein the fitness is increased via reduction of a competing bacteria or fungi, which may be neutral, commensal, or pathogenic.

149. A method of removing one or more undesirable materials from an environment comprising contacting the environment with an effective amount of an ADAS, such as an ADAS or composition of any one of paragraphs 1-84, wherein the ADAS comprises one or more molecules (such as proteins, polymers, nanoparticles, binding agents, or a combination thereof) that take up, chelate, or degrade the one or more undesirable materials.

150. The method of paragraph 149, where the undesirable material comprises a heavy metal, such as mercury, and the ADAS comprises one or more molecules (such as proteins, polymers, nanoparticles, binding agents, or a combination thereof) that bind heavy metals, such as MerR for mercury.

151. The method of paragraph 149, where the undesirable material comprises a plastic, such as PET, and the ADAS comprises one or more plastic degrading enzymes, such as PETase.

152. The method of paragraph 149, where the undesirable material comprises one or more small organic molecules and the ADAS comprise one or more enzymes capable of metabolizing said one or more small organic molecules.

153. A composition comprising a bacterium or ADAS, wherein the bacteria or ADAS comprises a T4SS, an RNA binding protein cargo, and an RNA cargo that is bound by the RNA binding protein and is suitable for delivery into a target cell through the T4SS.

154. The composition of paragraph 153, where the RNA binding protein is a Cas9 fused to VirE2 and VirF, the RNA cargo is a guide RNA, and, optionally, wherein the T4SS is the Ti system from Agrobacterium.

155. The composition of paragraph 153, where the RNA binding protein is p19 from Carnation Italian Ringspot Virus fused to VirE2 or VirF, the RNA cargo is an siRNA, and optionally wherein the T4SS is the Ti system from Agrobacterium.

5 156. A method of making the composition of paragraph 156, wherein a plasmid containing the Cas9 fused to VirE2 and VirF and RNA cargo are transfected into an Agrobacterium cell.

157. A method for delivering RNA to a plant cell or animal cell comprising contacting said plant cell or animal cell with a bacterium or ADAS, wherein the bacteria or ADAS comprises a T4SS, an RNA binding protein cargo, and an RNA cargo.

158. A method for delivering an RNA and a protein to a plant cell or animal cell comprising contacting said plant cell or animal cell with a bacterium or ADAS, wherein the bacteria or ADAS comprises a T4SS, an RNA binding protein cargo, and an RNA cargo.

1.-130. (canceled)

131. A method for delivering a cargo to a target cell, the method comprising:
(a) providing a composition comprising a plurality of highly active achromosomal dynamic active systems (ADAS), wherein the ADAS have an initial ATP concentration of at least 1.25 mM, the ADAS comprise a cargo, and the composition is substantially free of viable bacterial cells; and
(b) contacting the target cell with the composition of step (a) to deliver the cargo to the target cell.

132. The method of claim 131, wherein the target cell is an animal cell, a plant cell, or an insect cell.

133. The method of claim 131, wherein the cargo is a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP).

134. The method of claim 131, wherein the cargo includes a heterologous protein encoded by a plasmid in the ADAS.

135. The method of claim 131, wherein the cargo is encapsulated by the ADAS.

136. The method of claim 131, wherein the cargo is attached to the surface of the ADAS.

137. The method of claim 131, wherein the ADAS are derived from parent bacteria comprising a cell division topological specificity factor, and wherein the parent bacteria are Escherichia, Acinetobacter, Agrobacterium, Anabaena, Aquifex, Azoarcus, Azotobacter, Bordetella, Bradyrhizobium, Brucella, Buchnera, Burkholderia, Candidatus, Chromobacterium, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylococcus, Neisseria, Nitrosomonas, Nostoc, Photobacterium, Photorhabdus, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Symbiobacterium, or Thermoanaerobacter bacteria and the cell division topological specificity factor is the endogenous minE or DivIVA of the parent bacteria.

138. The method of claim 131, wherein providing includes reconstituting previously lyophilized ADAS.

139. The method of claim 138, wherein the reconstituted ADAS have an ATP concentration that is at least 95% of the ATP concentration of an ADAS that has not been lyophilized.

140. The method of claim 131, wherein the ADAS have an initial ATP concentration of at least 1.3 nM, 1.4 mM, 1.5 mM, 1.6 mM, 2 mM, 2.5 mM, 3 mM, 4 mM, 5 mM, 10 mM, 20 mM, 30 mM, or 50 mM.

141. A method for manufacturing a composition comprising a plurality of highly active achromosomal dynamic active systems (ADAS), wherein the composition is substantially free of viable bacterial cells, the method comprising:
(a) making, providing, or obtaining a plurality of parent bacteria having a reduction in the level or activity of a cell division topological specificity factor, wherein the reduction includes a reduction in expression of a minE, a minC, and a minD polypeptide;
(b) exposing the parent bacteria to conditions allowing the formation of minicells; and
(c) separating the minicells from the parent bacteria, thereby producing a composition comprising a plurality of ADAS that is substantially free of viable bacterial cells.

142. The method of claim 141, wherein an ATP concentration of the ADAS is increased following incubation at 37° C. for 12 hours.

143. The method of claim 141, wherein the parent bacteria have a deletion of the minCDE operon.

144. The method of claim 141, wherein the parent bacteria are Escherichia, Acinetobacter, Agrobacterium, Anabaena, Aquifex, Azoarcus, Azotobacter, Bordetella, Bradyrhizobium, Brucella, Buchnera, Burkholderia, Candidatus, Chromobacterium, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylococcus, Neisseria, Nitrosomonas, Nostoc, Photobacterium, Photorhabdus, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Symbiobacterium, or Thermoanaerobacter bacteria.

145. The method of claim 141, wherein the ADAS comprise a cargo.

146. The method of claim 145, wherein the cargo includes at least one of a nucleic acid, a plasmid, a polypeptide, a protein, an enzyme, an amino acid, a small molecule, a gene editing system, a hormone, an immune modulator, a carbohydrate, a lipid, an organic particle, an inorganic particle, and a ribonucleoprotein complex (RNP).

147. The method of claim 146, wherein the nucleic acid is a DNA, an RNA, or a plasmid.

148. The method of claim 145, wherein the cargo is expressed in the ADAS.

149. The method of claim 145, wherein the cargo is encapsulated by the ADAS.

150. A method of treating a plant in need thereof, the method comprising:
(a) providing a composition comprising a plurality of highly active achromosomal dynamic active systems (ADAS), wherein the ADAS have an initial ATP concentration of at least 1.25 mM, and wherein the composition is substantially free of viable bacterial cells; and
(b) contacting the plant or a pest thereof with an effective amount of the composition of step (a) to treat the plant.

151. The method of claim 150, wherein providing includes reconstituting previously lyophilized ADAS.

152. The method of claim 151, wherein the reconstituted ADAS have an ATP concentration that is at least 95% of the ATP concentration of an ADAS that has not been lyophilized.

153. The method of claim 150, wherein the ADAS carries a cargo protein.

154. The method of claim 150, wherein the cargo protein is a growth factor, an antibacterial protein, an antifungal protein, or an insecticidal protein.

155. A method of treating an animal in need thereof, the method comprising:
(a) providing a composition comprising a plurality of highly active achromosomal dynamic active systems (ADAS), wherein the ADAS have an initial ATP concentration of at least 1.25 mM, and wherein the composition is substantially free of viable bacterial cells; and
(b) contacting the animal with an effective amount of the composition of step (a) to treat the animal.

156. The method of claim 155, wherein the ADAS carries a chemotherapy cargo or an immunotherapy cargo.
